(19) United States
(12) Patent Application Publication
(10) Pub. No.: US 2015/0119363 A1
(43) Pub. Date: Apr. 30, 2015

(54) METHODS OF TREATING BACTERIAL INFECTIONS

(71) Applicant: Rampex Pharmaceuticals, Inc., San Diego, CA (US)

(72) Inventors: Michael N. Dudley, San Diego, CA (US); Scott Hecker, Del Mar, CA (US); Olga Rodny, Mill Valley, CA (US)

(73) Assignee: Rempex Pharmaceuticals, Inc.., San Diego, CA (US)

(21) Appl. No.: 14/377,163

(22) PCT Filed: Feb. 11, 2013
(86) PCT No.: PCT/US2013/025621

§ 371 Date: Aug. 6, 2014

Related U.S. Application Data
(60) Provisional application No. 61/599,148, filed on Feb. 15, 2012.

(51) Int. Cl. A61K 31/69; A61K 31/427; A61K 31/4196

(52) U.S. Cl. CPC A61K 31/69; A61K 31/4196; A61K 31/427
USPC 514/64

(57) ABSTRACT

The present invention relates to compounds, compositions and methods for treating bacterial infections. Embodiments of the present invention include antibiotics and β-lactamase inhibitors to treat resistant infections.

### RELATED APPLICATIONS

This application claims the benefit of U.S. Provisional Application No. 61/599,148 entitled “METHODS OF TREATING BACTERIAL INFECTIONS” filed on Feb. 15, 2012, the contents of which is incorporated herein by reference in its entirety.

### FIELD OF THE INVENTION

The present invention relates to compounds, compositions and methods for treating bacterial infections. Embodiments of the present invention include antibiotics and β-lactamase inhibitors to treat infections.

### BACKGROUND

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of anti-bacterial therapy. The increase in antibiotic resistant strains has been particularly common in major hospitals and care centers. The consequences of the increase in resistant strains include higher morbidity and mortality, longer patient hospitalization, and an increase in treatment costs

Various bacteria have evolved β-lactam deactivating enzymes, namely, β-lactamases, that counter the efficacy of the various β-lactams. β-lactamases can be grouped into 4 classes based on their amino acid sequences, namely, Ambler classes A, B, C, and D. Enzymes in classes A, C, and D include active-site serine β-lactamases, and class B enzymes, which are encountered less frequently, are Zn-dependent. These enzymes catalyze the chemical degradation of β-lactam antibiotics to render them inactive. Some β-lactamases can be transferred within and between various bacterial strains and species. The rapid spread of bacterial resistance and the evolution of multi-resistant strains severely limits β-lactam treatment options available.

The increase of class D β-lactamase-expressing bacterium strains such as Acinetobacter baumannii has become an emerging multidrug-resistant threat. A. baumannii strains express A, C, and D class β-lactamases. The class D β-lactamases such as the OXA families are particularly effective at destroying carbapenem type β-lactam antibiotics, e.g., imipenem, the active carbapenems component of Merck's Primaxin® (Montefour, K.; et al. Crit. Care Nurse 2008, 28, 15; Perez, F. et al. Expert Rev. Anti Infect. Ther. 2008, 6, 269; Bou, G.; Martinez-Beltran, J. Antimicrob. Agents Chemother. 2000, 40, 428. 2006, 50, 2280; Bou, G. et al, J. Antimicrob. Agents Chemother. 2000, 44, 1556). This has imposed a pressing threat to the effective use of drugs in that category to treat and prevent bacterial infections. Indeed the number of catalogued serine-based β-lactamases has exploded from less than ten in the 1970s to over 300 variants. These issues fostered the development of five “generations” of cephalosporins. When initially released into clinical practice, extended-spectrum cephalosporins resisted hydrolysis by the prevalent class A β-lactamases, TEM-1 and SHV-1. However, the development of resistant strains by the evolution of single or multiple amino acid substitutions in TEM-1 and SHV-1 resulted in the emergence of the extended-spectrum β-lactamase (ESBL) phenotype.

New β-lactamases have recently evolved that hydrolyze the carbapenem class of antimicrobials, including imipenem, biapenem, doripenem, meropenem, and ertapenem, as well as other β-lactam antibiotics. These carbapenemases belong to molecular classes A, B, and D. Class A carbapenemases of the KPC-type are predominantly in Klebsiella pneumoniae but now also reported in other Enterobacteriaceae, Pseudomonas aeruginosa and Acinetobacter baumannii. The KPC carbapenemase was first described in 1996 in North Carolina, but since then has disseminated widely in the US and Europe. Treatment of resistant strains with carbapenems can be associated with poor outcomes. β-lactamases of the Class B are metallobeta-lactamases and are characterized by use of a metal ion such as zinc for activity. Examples of Class B enzymes include VIM, IMP, and the recently described NDM-1 enzyme. These enzymes may be located in a variety of Gram-negative pathogens, including Enterobacteriaceae and Pseudomonas aeruginosa Older β-lactamase inhibitors such as tazobactam and clavulanic acid are ineffective against Class B enzymes, and have little or no inhibitory activity against Class C and Class D ezymes. While clavulanate and tazobactam have activity against some Class A beta-lactamases like TEM-1, they have lower activity against Class A carbapenemases (e.g,. KPC) as well as low activity against the chromosomal and plasmid-mediated Class C cephalosporinases and against many of the Class D oxacillinases. Therefore, there is a need for improved β-lactamase inhibitors.

### SUMMARY OF THE INVENTION

The present invention relates to compounds, compositions and methods for treating bacterial infections. Embodiments of the present invention include antibiotics and β-lactamase inhibitors to treat infections. Some embodiments include a method of increasing sensitivity of a bacterial infection to treatment with an antimicrobial β-lactam compound resistant to degradation by a metallo β-lactamase, said method comprising: identifying a bacterial infection as including bacteria that comprises a serine β-lactamase and a metallo β-lactamase; and contacting said bacteria with an effective amount of a β-lactamase inhibitor. In some embodiments, contacting said bacteria with an effective amount of a β-lactamase inhibitor comprises administering the β-lactamase inhibitor to a subject having said bacterial infection.

Some embodiments include a method of treating a bacterial infection that includes bacteria comprising a serine β-lactamase and a metallo β-lactamase, said method comprising: contacting said bacteria with a β-lactamase inhibiting effective amount of a β-lactamase inhibitor and an antibacterially effective amount of an antimicrobial β-lactam compound resistant to degradation by a metallo β-lactamase. Some embodiments also include identifying said bacterial infection as including bacteria that comprises a serine β-lactamase and a metallo β-lactamase. In some embodiments, contacting said bacteria with a β-lactamase inhibiting effective amount of a β-lactamase inhibitor and an antibacterially effective amount of an antimicrobial β-lactam compound resistant to degradation by a metallo β-lactamase comprises administering the β-lactamase inhibitor and the antimicrobial compound resistant to degradation by a metallo β-lactamase to a subject having said bacterial infection. In some embodiments, said administering comprises administering a pharmaceutical composition comprising said β-lactamase inhibitor and said antimicrobial compound resistant to degradation by a metallo β-lactamase to said subject.

Some embodiments include use of an antimicrobial β-lactam compound resistant to degradation by a metallo β-lactamase in the preparation of a medicament for use in combination with a β-lactamase inhibitor for treating a bacterial infection that includes bacteria comprising a serine β-lactamase and a metallo β-lactamase.

Some embodiments include use of a β-lactamase inhibitor in the preparation of a medicament for use in combination with an antimicrobial β-lactam compound resistant to degradation by a metallo β-lactamase for treating a bacterial infection that includes bacteria comprising a serine β-lactamase and a metallo β-lactamase.

Some embodiments include use of a β-lactamase inhibitor in the preparation of a medicament for increasing the sensitivity of a bacterial infection to an antimicrobial β-lactam compound resistant to degradation by a metallo β-lactamase, wherein the bacterial infection includes bacteria comprising a serine β-lactamase and a metallo β-lactamase.

In some embodiments, the antimicrobial compound resistant to degradation by a metallo β-lactamase has a Kₘ for the metallo β-lactamase greater than about 100 μM. In some embodiments, the antimicrobial compound resistant to degradation by a metallo β-lactamase has a Kₘ for the metallo β-lactamase greater than about 130 μM.

In some embodiments, the antimicrobial compound resistant to degradation by a metallo β-lactamase has a minimum inhibitory concentration for E. coli expressing the metallo β-lactamase less than about 250 μg/ml. In some embodiments, the antimicrobial compound resistant to degradation by a metallo β-lactamase has a minimum inhibitory concentration for E. coli expressing the metallo β-lactamase less than about 0.05 μg/ml.

In some embodiments, the antimicrobial compound resistant to degradation by a metallo β-lactamase comprises biapenem.

In some embodiments, the antimicrobial compound resistant to degradation by a metallo β-lactamase comprises a monobactam. In some embodiments, the antimicrobial compound resistant to degradation by a metallo β-lactamase is selected from the group consisting of Aztreonam, Tigemonam, Carumonam, SYN-2416, BA1.30072, and Nocardicin A.

In some embodiments, the sensitivity to the antimicrobial compound resistant to degradation by a metallo β-lactamase of the bacteria contacted with the β-lactamase inhibitor increases at least about 8-fold compared to bacteria not contacted with the β-lactamase inhibitor. In some embodiments, the sensitivity to the antimicrobial compound resistant to degradation by a metallo β-lactamase of the bacteria contacted with the β-lactamase inhibitor increases at least about 4-fold compared to bacteria not contacted with the β-lactamase inhibitor. In some embodiments, erein the sensitivity to the antimicrobial compound resistant to degradation by a metallo β-lactamase of the bacteria contacted with the β-lactamase inhibitor increases at least about 2-fold compared to bacteria not contacted with the β-lactamase inhibitor.

In some embodiments, the serine β-lactamase is selected from the group consisting of NMC-A, SME, KPC-2, OXA-48, and KPC-3. In some embodiments, the serine β-lactamase comprises a KPC enzyme. In some embodiments, the serine β-lactamase comprises KPC-2.

In some embodiments, the metallo β-lactamase comprises NDM-1.

In some embodiments, the metallo β-lactamase comprises IMP, VIM, SPM, and GIM.

In some embodiments, the bacterial infection comprises a bacterium selected from the group consisting of Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae. Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae. Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus. Staphylococcus hominis, and Staphylococcus saccharolyticus.

In some embodiments, a mammal has said bacterial infection. In some embodiments, a human has said bacterial infection.

### BRIEF DESCRIPTION OF THE DRAWINGS

FIG. 1 shows a graph of change in Log CFU/lung in a neutopenic mouse thigh infection model treated with Tigemonam alone or with the BLI, Compound A (also known as Compound 68).

### DETAILED DESCRIPTION

The present invention relates to compounds, compositions and methods for treating bacterial infections. Embodiments of the present invention include antibiotics and β-lactamase inhibitors to treat or prevent bacterial infections. Some embodiments include methods of treating or preventing a bacterial infection comprising administering a β-lactamase. Some such embodiments include contacting the bacteria causing the bacterial infection with a β-lactamase inhibitor and an antimicrobial β-lactam compound resistant to degradation by a metallo β-lactamase, such as a monobactam or biapenem. Some embodiments include identifying the bacterial infection as including a bacteria comprises a β-lactamase.

Some embodiments include methods of increasing the sensitivity of a bacterial infection to treatment with an antimicrobial β-lactam compound resistant to degradation by a metallo β-lactamase, such as a monobactam or biapenem. Some such embodiments include contacting the bacteria causing the infection with an effective amount of a β-lactamase inhibitor. Some such embodiments include indentifying a bacterial infection as including a bacteria that comprises a β-lactamase, and contacting the bacterial infection with an effective amount of a β-lactamase inhibitor. In some embodiments, the β-lactamase inhibitor increases the sensitivity of the bacteria in vitro and in vivo to an antimicrobial β-lactam compound resistant to degradation by a metallo β-lactamase, such as a monobactam or biapenem, compared to a bacterial infection not contacted with the β-lactamase inhibitor by at least about 2-fold, at least about 4-fold, at least about 8-fold, at least about 16-fold, and at least about 32-fold.

In some embodiments, the β-lactamase comprises a serine β-lactamase. In some embodiments, the β-lactamase comprises a metallo β-lactamase. In some embodiments, the bacteria comprises both a serine β-lactamase and a metallo β-lactamase. In preferred embodiments, the β-lactamase comprises a carbapenemase.

Examples of serine β-lactamases include KPC enzymes that are considered carbapenemases since they hydrolyze carbapenems as well as other beta-lactam antibiotics. Examples of KPC enzymes include KPC-2, KPC-3, KPC-3, KPC-4, KPC-5, KPC-6, KPC-7, KPC-8, KPC-9, KPC-10, and KPC-11 (see e.g., Bush, K. et al., (2010) Antimicro. Agents & Chemo. 54:969-976, incorporated by reference in its entirety). In some embodiments, the serine β-lactamases is NMC-A, SME, KPC-2, OXA-48, and KPC-3. In some embodiments, the serine β-lactamases is KPC-2. Examples of metallo β-lactamases include NDM-1, IMP, VIM, SPM, and GIM (see e.g., Walsh T. R., et al., (2005) Am. Soc. Micro. 18:306-325, incorporated herein by reference in its entrirety). In some embodiments, the metallo β-lactamase comprises NDM-1.

Methods of identifying a bacterial infection as including bacteria that comprise a β-lactamase, including one or more particular β-lactamases, are well known in the art. Examples of identifying a bacterial infection as including bacteria that comprise a β-lactamase include PCR and phenotypic tests, including screens based on media such as ChromlD ESBL culture medium (see e.g., Nordmann P. et al., (2011) J. Clin. Micro. 49:718-721, incorporated herein by reference in its entirety).

### DEFINITIONS

Terms and substituents are given their ordinary meaning unless defined otherwise, and may be defined when introduced and retain their definitions throughout unless otherwise specified, and retain their definitions whether alone or as part of another group unless otherwise specified.

As used herein, “alkyl” means a branched, or straight chain saturated chemical group containing only carbon and hydrogen, such as methyl, isopropyl, isobutyl, sec-butyl and pentyl. In various embodiments, alkyl groups can either be unsubstituted or substituted with one or more substituents, e.g., halogen, hydroxyl, substituted hydroxyl, acyloxy, amino, substituted amino, amido, cyano, nitro, guanidino, amidino, mercapto, substituted mercapto, carboxy, sulfonyloxy, carbonyl, benzyloxy, aryl, heteroaryl, carbocyclyl, heterocyclyl, or other functionality that may be suitably blocked with a protecting group. Typically, alkyl groups will comprise 1 to 20 carbon atoms, 1 to 9 carbon atoms, preferably 1 to 6, and more preferably 1 to 5 carbon atoms.

As used herein, “alkenyl” means a straight or branched chain chemical group containing only carbon and hydrogen and containing at least one carbon-carbon double bond, such as 1-propenyl, 2-propenyl, 2-methyl-1-propenyl, 1-butenyl, 2-butenyl, and the like. In various embodiments, alkenyls can either be unsubstituted or substituted with one or more substituents, e.g., halogen, hydroxyl, substituted hydroxyl, acyloxy, amino, substituted amino, amido, cyano, nitro, guanidino, amidino, mercapto, substituted mercapto, carboxy, sulfonyloxy, carbonyl, benzyloxy, aryl, heteroaryl, carbocyclyl, heterocyclyl, or other functionality that may be suitably blocked with a protecting group. Typically, alkenyl groups will comprise 2 to 20 carbon atoms, 2 to 9 carbon atoms, preferably 2 to 6, and more preferably 2 to 5 carbon atoms.

As used herein, “alkynyl” means a straight or branched chain chemical group containing only carbon and hydrogen and containing at least one carbon-carbon triple bond, such as 1-propynyl, 1-butynyl, 2-butynyl, and the like. In various embodiments, alkynyls can either be unsubstituted or substituted with one or more substituents, e.g., halogen, hydroxyl, substituted hydroxyl, acyloxy, amino, substituted amino, amido, cyano, nitro, guanidino, amidino, mercapto, substituted mercapto, carboxy, sulfonyloxy, carbonyl, benzyloxy, aryl, heteroaryl, carbocyclyl, heterocyclyl, or other functionality that may be suitably blocked with a protecting group. Typically, alkynyl groups will comprise 2 to 20 carbon atoms, 2 to 9 carbon atoms, preferably 2 to 6, and more preferably 2 to 5 carbon atoms. [0130] As used herein, “carbocyclyl” means a non-aromatic cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that at least one ring in the ring system is not aromatic. In various embodiments, carbocyclyl groups can either be unsubstituted or substituted with one or more substituents, e.g., halogen, alkoxy, acyloxy, amino, amido, cyano, nitro, hydroxyl, mercapto, carboxy, carbonyl, benzyloxy, aryl, heteroaryl, or other functionality that may be suitably blocked with a protecting group. Typically, carbocyclyl groups will comprise 3 to 10 carbon atoms, preferably 3 to 6.

As used herein, “cycloalkyl” means a fully saturated carbocyclyl ring system. Examples include cyclopropyl, cyclobutyl, cyclopentyl, and cyclohexyl.

As used herein, “cycloalkenyl” means a carbocyclyl ring system having at least one double bond. An example is cyclohexenyl.

As used herein, “lower alkyl” means a subset of alkyl, and thus is a hydrocarbon substituent, which is linear, or branched. Preferred lower alkyls are of 1 to about 4 carbons, and may be branched or linear. Examples of lower alkyl include butyl, propyl, isopropyl, ethyl, and methyl. Likewise, radicals using the terminology “lower” refer to radicals preferably with 1 to about 4 carbons in the alkyl portion of the radical.

As used herein, “aryl” means an aromatic radical having a single-ring (e.g., phenyl) or multiple condensed rings (e.g., naphthyl or anthryl) with only carbon atoms present in the ring backbone. In various embodiments, aryl groups can either be unsubstituted or substituted with one or more substituents, e.g., amino, cyano, hydroxyl, lower alkyl, haloalkyl, alkoxy, nitro, halo, mercapto, carboxy, carbonyl, benzyloxy, aryl, heteroaryl, and other substituents. Some embodiments include substitution with an alkoxy group, which may be further substituted with one or more substituents, e.g., amino, cyano, hydroxyl, lower alkyl, haloalkyl, alkoxy, nitro, halo, mercapto, and other substituents. A preferred aryl is phenyl.

As used herein, the term “heteroaryl” means an aromatic radical having one or more heteroatom(s) (e.g., N, O, or S) in the ring backbone and may include a single ring (e.g., pyridine) or multiple condensed rings (e.g., quinoline). In various embodiments, heteroaryl groups can either be unsubstituted or substituted with one or more substituents, e.g., amino, cyano, hydroxyl, lower alkyl, haloalkyl, alkoxy, nitro, halo, mercapto, carboxy, carbonyl, benzyloxy, aryl, heteroaryl, and other substituents. Examples of heteroaryl include thienyl, pyrridyl, furyl, oxazolyl, oxadiazolyl, pyrollyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl, quinolinyl, quinazolinyl and others.

In these definitions it is contemplated that substitution on the aryl and heteroaryl rings is within the scope of certain embodiments. Where substitution occurs, the radical is called substituted aryl or substituted heteroaryl. Preferably one to three and more preferably one or two substituents occur on the aryl ring. Though many substituents will be useful, preferred substituents include those commonly found in aryl compounds, such as alkyl, cycloalkyl, hydroxy, alkoxy, cyano, halo, haloalkyl, mercapto and the like.

As used herein, “amide” or “amido” includes both RNR′CO— (in the case of R=alkyl, alkaminocarbonyl-) and RCONR′— (in the case of R=alkyl, alkyl carbonylamino-). “Amide” or “amido” includes a H—CON—, alkyl-CON—, carbocyclyl-CON—, aryl-CON—, heteroaryl-CON— or heterocyclyl-CON— group, wherein the alkyl, carbocyclyl, aryl or heterocyclyl group is as herein described.

As used herein, the term “ester” includes both ROCO— (in the case of R=alkyl, alkoxycarbonyl-) and RCOO— (in the case of R=alkyl, alkylcarbonyloxy-).

As used herein, “acyl” means an H—CO—, alkyl-CO—, carbocyclyl-CO—, aryl-CO—, heteroaryl-CO— or heterocyclyl-CO— group wherein the alkyl, carbocyclyl, aryl or heterocyclyl group is as herein described. Preferred acyls contain a lower alkyl. Exemplary alkyl acyl groups include formyl, acetyl, propanoyl, 2-methylpropanoyl, t-butylacetyl, butanoyl and palmitoyl.

As used herein, “halo or halide” is a chloro, bromo, fluoro or iodo atom radical. Chloro and fluoro are preferred halides. The term “halo” also contemplates terms sometimes referred to as “halogen”, or “halide”.

As used herein, “heterocyclyl” means a non-aromatic cyclic ring system comprising at least one heteroatom in the ring system backbone. Heterocyclyls may include multiple fused rings. Heterocyclyls may have any degree of saturation provided that at least one ring in the ring system is not aromatic. The heteroatom(s) may be present in either a non-aromatic or aromatic ring in the ring system. In various embodiments, heterocyclyls may be substituted or unsubstituted with one or more substituents, e.g., halogen, alkoxy, acyloxy, amino, amido, cyano, nitro, hydroxyl, mercapto, carboxy, carbonyl, benzyloxy, aryl, heteroaryl, and other substituents, and are attached to other groups via any available valence, preferably any available carbon or nitrogen. Preferred heterocycles are of 5-7 members. In six membered monocyclic heterocycles, the heteroatom(s) are selected from one up to three of 0, N or S, and when the heterocycle is five membered, preferably it has one or two heteroatoms selected from O, N, or S. Examples of heterocyclyl include pyrrolidinyl, piperidinyl, azepanyl, tetrahydrofuranyl, tetrahydropyranyl, oxepanyl, tetrahydrothiophenyl, tetrahydrothiopyranyl, thiepanyl, indolinyl and dihydrobenzofuranyl.

As used herein, “substituted amino” means an amino radical which is substituted by one or two alkyl, cycloalkyl, aryl, heteroaryl or heterocyclyl groups, wherein the alkyl, aryl, heteroaryl, cycloalkyl or heterocyclyl are defined as above.

As used herein, “substituted hydroxyl” means RO— group wherein R is an alkyl, an aryl, heteroaryl, cycloalkyl or a heterocyclyl group, wherein the alkyl, cycloalkyl, aryl, heteroaryl or heterocyclyl are defined as above.

As used herein, “substituted thiol” means RS— group wherein R is an alkyl, an aryl, heteroaryl, cycloalkyl or a heterocyclyl group, wherein the alkyl, cycloalkyl, aryl, heteroaryl or heterocyclyl are defined as above.

As used herein, “sulfonyl” means an alkylSO₂, arylSO₂, heteroarylSO₂, carbocyclylSO₂, or heterocyclyl-SO₂ group wherein the alkyl, carbocyclyl, aryl, heteroaryl or heterocyclyl are defined as above.

As used herein, “sulfamido” means an alkyl-N—S(O)₂N—, aryl-NS(O)₂N—, heteroaryl-NS(O)₂N—, carbocyclyl-NS(O)₂N or heterocyclyl-NS(O)₂N— group wherein the alkyl, carbocyclyl, aryl, heteroaryl or heterocyclyl group is as herein described.

As used herein, “sulfonamido” means an alkyl-S(O)₂N—, aryl-S(O)₂N—, heteroaryl-S(O)₂N—, carbocyclyl-S(O)₂N— or heterocyclyl-S(O)₂N— group wherein the alkyl, carbocyclyl, aryl, heteroaryl or heterocyclyl group is as herein described.

As used herein, “ureido” means an alkyl-NCON—, aryl-NCON—, heteroaryl-NCON—, carbocyclyl-NCON—, heterocyclyl-NCON— group or heterocyclyl-CON— group wherein the heterocyclyl group is attached by a ring nitrogen, and wherein the alkyl, carbocyclyl, aryl, heteroaryl or heterocyclyl group is as herein described.

As used herein, “guanidino” means an alkyl-NC(═NR′)N—, aryl-NC(═NR′)N—, heteroaryl-NC(═NR′)N—, carbocyclyl-NC(═NR′)N— or heterocyclyl-NC(═NR′)N— group wherein R′ is an H, substituted or unsubstituted hydroxyl, CN, alkyl, aryl, heteroaryl or a heterocyclyl group, wherein the alkyl, carbocyclyl, aryl, heteroaryl or heterocyclyl group is as herein described.

As used herein, a substituted group is derived from the unsubstituted parent group in which there has been an exchange of one or more hydrogen atoms for another atom or group. When substituted, the substituent group(s) is (are) substituted with one or more substituent(s) individually and independently selected from C₁-C₆ alkyl, C₁-C₆ alkenyl, C₁-C₆ alkynyl, C₃-C₇ carbocycle (optionally substituted with halo, alkyl, alkoxy, carboxyl, haloalkyl, CN, —SO₂-alkyl, —CF₃, and —OCF₃), C₁-C₆ heteroalkyl, 5-7 membered heterocyclyl (e.g., tetrahydrofuryl) (optionally substituted with halo, alkyl, alkoxy, carboxyl, CN, —SO₂-alkyl, —CF₃, and —OCF₃), aryl (optionally substituted with halo, alkyl, aryl optionally substituted with C₁-C₆ alkyl, arylalkyl, alkoxy, carboxyl, CN, —SO₂-alkyl, —CF₃, and —OCF₃), arylalkyl (optionally substituted with halo, alkyl, alkoxy, aryl, carboxyl, CN, —SO₂-alkyl, —CF₃, and —OCF₃), heteroaryl (optionally substituted with halo, alkyl, alkoxy, aryl, aralkyl, carboxyl, CN, —SO₂-alkyl, —CF₃, and —OCF₃), heteroarylalkyl (optionally substituted with halo, alkyl, alkoxy, aryl, carboxyl, CN, —SO₂-alkyl, —CF₃, and —OCF₃), halo (e.g., chloro, bromo, iodo and fluoro), cyano, hydroxy, C₁-C₆ alkoxy, C₁-C₆ alkoxyalkyl (i.e., ether), aryloxy, sulfhydryl(mercapto), halo(C₁-C₆)alkyl (e.g., —CF₃), C₁-C₆ alkylthio, arylthio, amino (—NH₂), mono- and di-(C₁-C₆)alkyl amino, quaternary ammonium salts, amino(C₁-C₆)alkoxy (e.g, —O(CH₂)₄NH₂), amino(C₁-C₆)alkoxyalkyl (e.g., —CH₂O(CH₂)₂NH₂), hydroxy(C₁-C₆)alkylamino, amino(C₁-C₆)alkylthio (e.g, —S(CH₂)₂NH₂), cyanoamino, nitro, carbamyl, oxo (═O), carboxy, glycolyl, glycyl, hydrazino, guanidinyl, sulfamyl, sulfonyl, sulfinyl, thiocarbonyl, thiocarboxy, C-amide, N-amide, N-carbamate, O-carbamate, and urea. Wherever a group is described as “optionally substituted” that group can be substituted with the above substituents.

In some embodiments, substituted group(s) is (are) substituted with one or more substituent(s) individually and independently selected from C₁-C₆ alkyl, C₃-C₇ carbocycle, amino (—NH₂), amino(C₁-C₆)alkoxy, carboxyl, oxo (═O), C₁-C₆ alkylthio, amino(C₁-C₆)alkylthio, guanidinyl, aryl, 5-7 membered heterocyclyl, heteroarylalkyl, hydroxy, halo, amino(C₁-C₆)alkoxy, and amino(C₁-C₆)alkoxyalkyl.

In some embodiments, substituted group(s) is (are) substituted with one or more substituent(s) individually and independently selected from C₁-C₆ alkyl, amino (—NH₂), amino(C₁-C₆)alkoxy, carboxyl, oxo (═O), C₁-C₆ alkylthio, amino(C₁-C₆)alkylthio, guanidinyl, hydroxy, halo, amino(C₁-C₆)alkoxy, and amino(C₁-C₆)alkoxyalkyl.

In some embodiments, substituted group(s) is (are) substituted with one or more substituent(s) individually and independently selected from C₁-C₆ alkyl, amino (—NH₂), carboxyl, oxo (═O), guanidinyl, hydroxy, and halo.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical. For example, as used herein, “alkylene” means a branched, or straight chain saturated di-radical chemical group containing only carbon and hydrogen, such as methylene, isopropylene, isobutylene, sec-butylene, and pentylene, that is attached to the rest of the molecule via two points of attachment. As used herein, “alkenylene” means a straight or branched chain di-radical chemical group containing only carbon and hydrogen and containing at least one carbon-carbon double bond, such as 1-propenylene, 2-propenylene, 2-methyl-1-propenylene, 1-butenylene, and 2-butenylene, that is attached to the rest of the molecule via two points of attachment.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR⁹, —PO₂(R⁹)₂, —PO₃(R⁹)₂, —CONHNHSO₂R⁹, —COHNSO₂R⁹, and —CONR⁹CN, where R⁹ is as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R⁹ as defined herein.

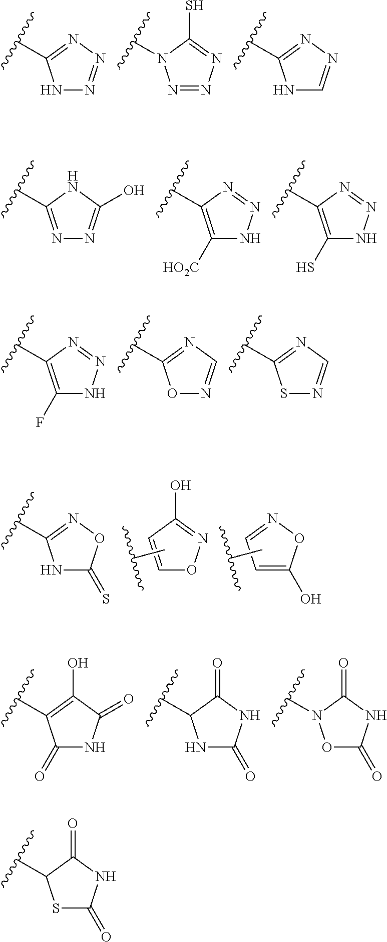

It is also contemplated that when chemical substituents are added to a carboxylic isostere, the compound retains the properties of a carboxylic isostere. It is contemplated that when a carboxylic isostere is optionally substituted with one or more moieties selected from R⁹ as defined herein, then the substitution and substitution position is selected such that it does not eliminate the carboxylic acid isosteric properties of the compound. Similarly, it is also contemplated that the placement of one or more R⁹ substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

The skilled artisan will recognize that some structures described herein may be resonance forms or tautomers of compounds that may be fairly represented by other chemical structures, even when kinetically; the artisan recognizes that such structures are only a very small portion of a sample of such compound(s). Such compounds are considered within the scope of the structures depicted, though such resonance forms or tautomers are not represented herein.

In some embodiments, due to the facile exchange of boron esters, some of the compounds described herein may convert to or exist in equilibrium with alternate forms. Accordingly, in some embodiments, the compounds described herein may exist in combination with one or more of these forms. For example, Compound 5 may exist in combination with one or more open-chain form (5a), dimeric form (5b), cyclic dimeric form (5c), trimeric form (5d), cyclic trimeric form (5e), and the like.

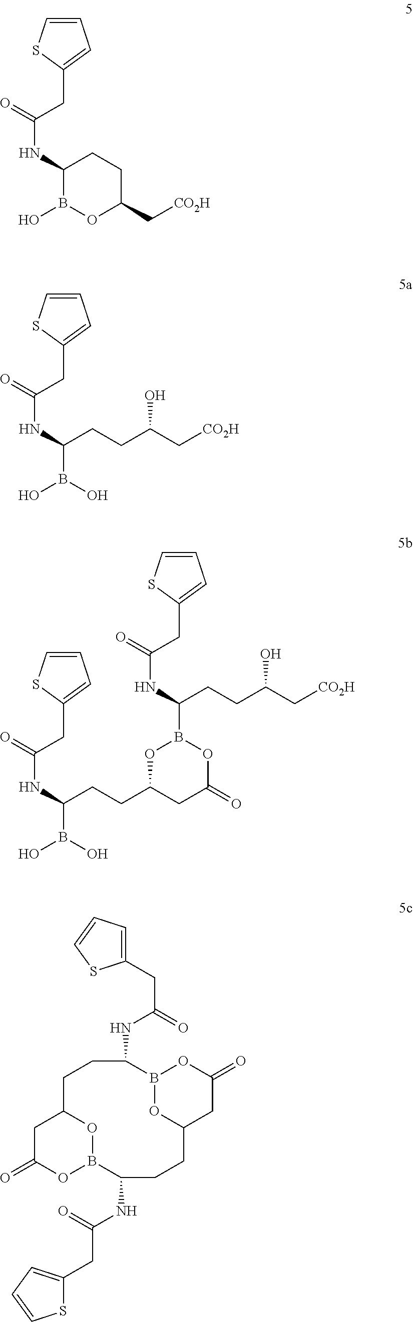 [SMILES: O=C(O)C[C@@H]1CC[C@H](NC(=O)Cc2cccs2)B(O)O1 ; O=C(O)C[C@@H](O)CC[C@H](NC(=O)Cc1cccs1)B(O)O ; O=C(O)C[C@@H](O)CC[C@H](NC(=O)Cc1cccs1)B1OC(=O)C[C@H](CC[C@H](NC(=O)Cc2cccs2)B(O)O)O1 ; O=C(Cc1cccs1)N[C@H]1CCC2CC(=O)OB(O2)[C@@H](NC(=O)Cc2cccs2)CCC2CC(=O)OB1O2]

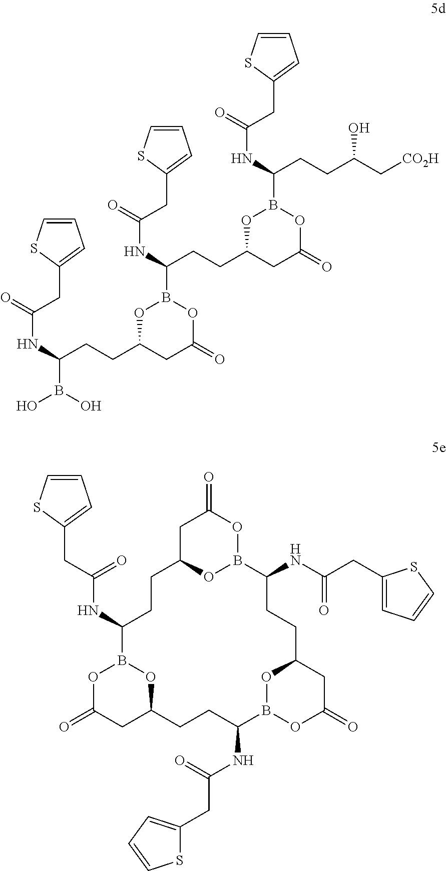 [SMILES: O=C(O)C[C@@H](O)CC[C@H](NC(=O)Cc1cccs1)B1OC(=O)C[C@H](CC[C@H](NC(=O)Cc2cccs2)B2OC(=O)C[C@H](CC[C@H](NC(=O)Cc3cccs3)B(O)O)O2)O1 ; O=C(Cc1cccs1)N[C@H]1CC[C@H]2CC(=O)OB(O2)[C@@H](NC(=O)Cc2cccs2)CC[C@H]2CC(=O)OB(O2)[C@@H](NC(=O)Cc2cccs2)CC[C@H]2CC(=O)OB1O2]

The compounds provided herein may encompass various stereochemical forms. The compounds also encompasses diastereomers as well as optical isomers, e.g. mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes humans, cattle, horses, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

The term “pharmaceutically acceptable salt” refers to salts that retain the biological effectiveness and properties of the compounds of the preferred embodiments and, which are not biologically or otherwise undesirable. In many cases, the compounds of the preferred embodiments are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Solvate” refers to the compound formed by the interaction of a solvent and an EPI, a metabolite, or salt thereof. Suitable solvates are pharmaceutically acceptable solvates including hydrates.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

A therapeutic effect relieves, to some extent, one or more of the symptoms of the infection, and includes curing an infection. “Curing” means that the symptoms of active infection are eliminated, including the elimination of excessive members of viable microbe of those involved in the infection. However, certain long-term or permanent effects of the infection may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment,” “prevent,” “prevention,” or “preventing” refers to treating a patient who is not yet infected, but who is susceptible to, or otherwise at risk of, a particular infection, whereby the treatment reduces the likelihood that the patient will develop an infection. The term “therapeutic treatment” refers to administering treatment to a patient already suffering from an infection.

### Administration and Pharmaceutical Compositions

Some embodiments include pharmaceutical compositions comprising: (a) a safe and therapeutically effective amount of a beta lactamase inhibitor provided herein; and (b) a pharmaceutically acceptable carrier. In some embodiments, the pharmaceutical composition additionally comprises an antimicrobial β-lactam compound resistant to degradation by a metallo β-lactamase, such as a monobactam or biapenem.

The β-lactamase inhibitors are administered at a therapeutically effective dosage, e.g., a dosage sufficient to inihibit the β-lactamase to a level sufficient to provide treatment or prevention of a bacterial infection when used in combination with an antibiotic such as an antimicrobial β-lactam compound resistant to degradation by a metallo β-lactamase, such as a monobactam or biapenem biapenem. While human dosage levels have yet to be optimized for the compounds of the preferred embodiments, generally, a daily dose for most of the β-lactamase inhibitors described herein is from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

Standard pharmaceutical formulation techniques are used, such as those disclosed in Remington's The Science and Practice of Pharmacy, 21st Ed., Lippincott Williams & Wilkins (2005), incorporated by reference in its entirety.

In addition to the active ingredients described above, come embodiments include compositions containing a pharmaceutically-acceptable carrier. The term “pharmaceutically-acceptable carrier”, as used herein, means one or more compatible solid or liquid filler diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound or compounds that are suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day, or as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. These oral forms comprise a safe and effective amount, usually at least about 5%, with a maximum of about 90%, of the active ingredients. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH. In various embodiments, the pH of the final composition ranges from 2 to 8, or preferably from 4 to 7. The resulting composition may be infused into the patient over a period of time. In various embodiments, the infusion time ranges from 5 minutes to continuous infusion, from 10 minutes to 8 hours, from 30 minutes to 4 hours, and from 1 hour to 3 hours. In one embodiment, the drug is infused over a 3 hour period. The infusion may be repeated at the desired dose interval, which may include, for example, 6 hours, 8 hours, 12 hours, or 24 hours.

The compositions for intravenous administration may be provided to caregivers in the form of one more solids that are reconstituted shortly prior to administration. In other embodiments, the compositions are provided in solution ready to administer. In still other embodiments, the compositions are provided in a solution that is further diluted prior to administration. In embodiments that include administering a β-lactamase inhibitor in combination with an antimicrobial β-lactam compound resistant to degradation by a metallo β-lactamase, such as a monobactam or biapenem, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

### Methods of Treatment

Some embodiments of the present invention include methods of treating bacterial infections with the compounds and compositions comprising β-lactamase inhibitors described herein. Some methods include administering a compound, composition, pharmaceutical composition described herein to a subject in need thereof. In some embodiments, a subject can be an animal, e.g., a mammal, a human. In some embodiments, the bacterial infection comprises a bacteria described herein. As will be appreciated from the foregoing, methods of treating a bacterial infection include methods for preventing bacterial infection in a subject at risk thereof.

Some embodiments include co-administering a β-lactamase inhibitor with an antimicrobial β-lactam compound resistant to degradation by a metallo β-lactamase, such as a monobactam or biapenem. As used herein “an antimicrobial β-lactam compound resistant to degradation by a metallo β-lactamase” includes an antimicrobial compound that is relatively resistant to hydolysis by a metallo β-lactamase compared to an antimicrobial compound that is hydrolyzed by a metallo β-lactamase. For example, in some embodiments, the Kₘ of an antimicrobial β-lactam compound resistant to degradation by a metallo β-lactamase with a metallo β-lactamase, such as NDM-1, can be at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 110 μM, at least about 120 μM, at least about 130 μM, at least about 140 μM, at least about 150 μM, at least about 160 μM, at least about 170 μM, at least about 180 μM, at least about 190 μM, and at least about 100 μM. In some embodiments, the Kₘ of an antimicrobial β-lactam compound resistant to degradation by a metallo β-lactamase with a metallo β-lactamase, such as NDM-1, can be at least about 150 at least about 200 at least about 250 at least about 300 at least about 350 at least about 400 at least about 450 at least about 500 μM.

In some embodiments, an antimicrobial β-lactam compound resistant to degradation by a metallo β-lactamase includes an antimicrobial compound with a metallo β-lactamase having a k_{cat} of at least about 50 s⁻¹, 100 s⁻¹, 150 s⁻¹, 200 s⁻¹, 250 s⁻¹, 300 s⁻¹, 350 s⁻¹, 400 s⁻¹, 450 s⁻¹, and 500 s⁻¹.

In some embodiments, an antimicrobial β-lactam compound resistant to degradation by a metallo β-lactamase includes an antimicrobial compound with a minimum inhibitory concentration (MIC) against a pathogenic microorganism expressing a metallo β-lactamase, such as NMD-1, such as Klebsiella spp. and E. coli, or Pseudomonas aeruginosa, less than about 300 μg/ml, less than about 250 μg/ml, less than about 200 μg/ml, less than about 150 μg/ml, less than about 100 μg/ml, less than about 50 μg/ml. In some embodiments, an antimicrobial β-lactam compound resistant to degradation by a metallo β-lactamase includes an antimicrobial compound with a MIC against a pathogenic microorganism expressing a metallo β-lactamase, such as NMD-1, less than about 50 μg/ml, less than about 40 μg/ml, less than about 30 μg/ml, less than about 20 μg/ml, less than about 10 μg/ml, and less than about 1 μg/ml. In some embodiments, an antimicrobial β-lactam compound resistant to degradation by a metallo β-lactamase includes an antimicrobial compound with a MIC against a pathogenic microorganism expressing a metallo β-lactamase, such as NMD-1, less than about 1.00 μg/ml, less than about 0.90 μg/ml, less than about 0.80 μg/ml, less than about 0.70 μg/ml, less than about 0.60 μg/ml, less than about 0.50 μg/ml, less than about 0.40 μg/ml, less than about 0.30 μg/ml, less than about 0.20 μg/ml, and less than about 0.10 μg/ml. In some embodiments, an antimicrobial compound resistant to degradation by a metallo β-lactamase, such as NDM-1, includes an antimicrobial compound with a MIC against a pathogenic microorganism expressing a metallo β-lactamase, such as NMD-1, less than about 0.10 μg/ml, less than about 0.09 μg/ml, less than about 0.08 μg/ml, less than about 0.07 μg/ml, less than about 0.06 μg/ml, less than about 0.05 μg/ml, less than about 0.04 μg/ml, less than about 0.03 μg/ml, less than about 0.02 μg/ml, and less than about 0.01 μg/ml. In some of the foregoing embodiments, the pathogenic microorganism comprises a single metallo β-lactamase. In some of the foregoing embodiments, the pathogenic microorganism comprises more than one metallo β-lactamase.

Examples of monobactams include SYN-2416 (also known as PTX2416), BA1.30072, Aztreonam, Tigemonam, and Carumonam, the structures of which are:

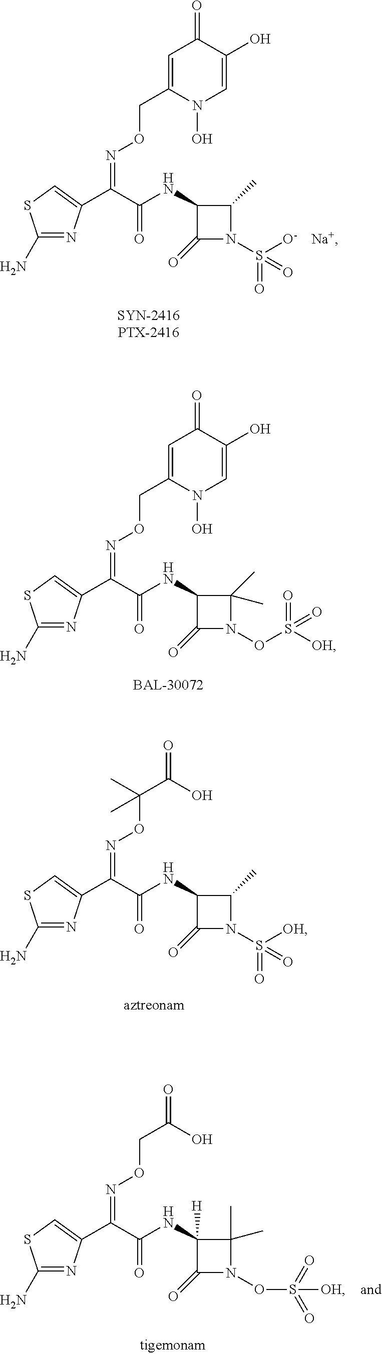

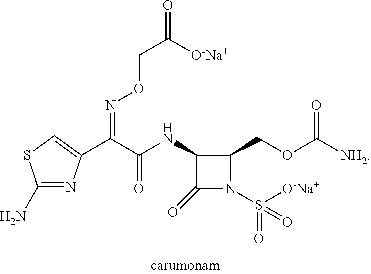

Some embodiments include an antimicrobial compound useful with the methods, compositions and compounds provided herein includes Nocardicin A, having the following formula:

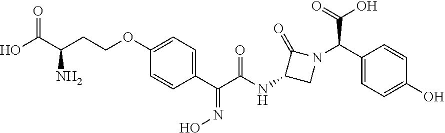 [SMILES: N[C@H](CCOc1ccc(/C(=N\O)C(=O)N[C@H]2CN([C@@H](C(=O)O)c3ccc(O)cc3)C2=O)cc1)C(=O)O]

Some embodiments include an antimicrobial compound useful with the methods, compositions and compounds provided herein are described in Int. Pub. WO2008116813, incorporated herein by reference in its entirety. Some embodiments include compounds having the following formula:

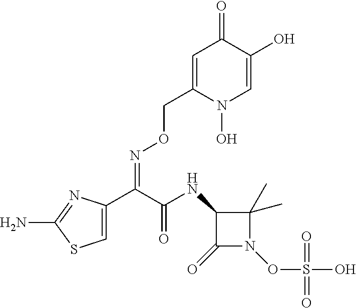

wherein, the oxyimino group i.e. >C═N—O— has Z-orientation, or a pharmaceutically acceptable salt thereof.

Some embodiments may also include co-administering additional medicinal agents. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. When combining the agents in a single dosage form, they may be physically mixed (e.g, by co-dissolution or dry mixing) or may form an adduct or be covalently linked such that they split into the two or more active ingredients upon administration to the patient. In another embodiment, the agents are administered sequentially. In one embodiment the agents are administered through the same route, such as orally. In another embodiment, the agents are administered through different routes, such as one being administered orally and another being administered i.v.

### Indications

The compounds and compositions comprising β-lactamase inhibitors described herein and their combinations with an antimicrobial β-lactam compound resistant to degradation by a metallo β-lactamase, such as a monobactam or biapenem, can be used to treat bacterial infections. Bacterial infections that can be treated with the compounds, compositions and methods described herein can comprise a wide spectrum of bacteria. Example organisms include gram-positive bacteria, gram-negative bacteria, aerobic and anaerobic bacteria, such as Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms.

More examples of bacterial infections include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

β-lactamase Inhibitors

Some β-lactamase inhibitors useful with the methods, compositions and compounds provided herein are described in PCT/US2011/046957, incorporated herein by reference in its entirety. Some embodiments include a compound having the structure of formula (I):

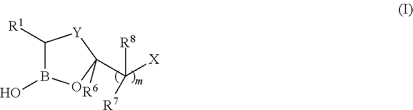

or a pharmaceutically acceptable salt thereof,

wherein, Y is a 1-4 atom alkylene or 2-4 atom alkenylene linker, optionally substituted by one or more substituents selected from the group consisting of Cl, F, CN, CF₃, —R⁹, —OR⁹, —C(═O)NR⁹R¹⁰, and —C(═O)OR⁹, wherein said alkylene or alkenylene linker is optionally fused to an optionally substituted aryl, optionally substituted heteroaryl, optionally substituted carbocyclyl, or optionally substituted heterocyclyl;

R¹ is selected from a group consisting of —C₁₋₉alkyl, —C₂₋₉alkenyl, —C₂₋₉alkynyl, —NR⁹R¹⁰, —C₁₋₉alkylR¹¹, —C₂₋₉alkenylR¹¹, —C₂₋₉alkynylR¹¹, -carbocyclyl-R¹¹, —CH(OH)C₁₋₉alkylR⁹, —CH(OH)C₂₋₉alkenylR⁹, —CH(OH)C₂₋₉alkynylR⁹, —CH(OH)carbocyclyl-R⁹, —C(═O)R⁹, —C(═O)C₁₋₉alkylR⁹, —C(═O)C₂₋₉alkenylR⁹, —C(═O)C₂₋₉alkynylR⁹, —C(═O)C₂₋₉carbocyclyl-R⁹, —C(═O)NR⁹R¹⁰, —N(R⁹)C(═O)R⁹, —N(R⁹)C(═O)NR⁹R¹⁰, —N(R⁹)C(═O)OR⁹, —N(R⁹)C(═O)C(═NR¹⁰)R⁹, —N(R⁹)C(═O)C(═NOR¹⁰)R⁹, —N(R⁹)C(═O)C(═CR⁹R¹⁰)R⁹, —N(R⁹)C(═O)C₁₋₄alkylN(R⁹)C(═O)R⁹, —N(R⁹)C(═NR¹⁰)R⁹, —C(═NR¹⁰)NR⁹R¹⁰, —N═C(R⁹)NR⁹R¹⁰, —N(R⁹)SO₂R⁹, —N(R⁹)SO₂NR⁹R¹⁰, —N═CHR⁹, substituted or unsubstituted aryl, substituted or unsubstituted heteroaryl, substituted or unsubstituted carbocyclyl, and substituted or unsubstituted heterocyclyl;

R⁶ is selected from a group consisting of H, C₂₋₉alkenyl, —C₂₋₉alkynyl, carbocyclyl, —C₁₋₉alkylR¹¹, —C₂₋₉alkenylR¹¹, —C₂₋₉alkynylR¹¹, carbocyclyl-R¹¹, —C(═O)OR⁹, —C₁₋₉alkylCO₂R⁹, —C₂₋₉alkenylCO₂R⁹, —C₂₋₉alkynylCO₂R⁹, and -carbocyclyl-CO₂R⁹, or alternatively R⁶ and an R⁷ are taken together with the atoms to which they are attached to form a substituted or unsubstituted carbocyclyl or substituted or unsubstituted heterocyclyl, or alternatively R⁶ and a carbon atom in Y are taken together with intervening atoms to form a substituted or unsubstituted carbocyclyl or substituted or unsubstituted heterocyclyl;

each R⁷ is independently selected from a group consisting of H, —NR⁹R¹⁰, —OR⁹, —C₁₋₉alkylCO₂R⁹, —C₂₋₉alkenylCO₂R⁹, —C₂₋₉alkynylCO₂R⁹, and -carbocyclyl-CO₂R⁹, or independently, R⁶ and an R⁷ or independently, an R⁷ and an R⁸ are taken together with the atoms to which they are attached to form a substituted or unsubstituted carbocyclyl or substituted or unsubstituted heterocyclyl, or independently, an R⁷ and a carbon atom in Y are taken together with intervening atoms to form a substituted or unsubstituted carbocyclyl or substituted or unsubstituted heterocyclyl, or independently a geminal R⁷ and R⁸ together form a C₂₋₉ alkenylenylCO₂R⁹;

each R⁸ is independently selected from a group consisting of H, —NR⁹R¹⁰, —OR⁹, —C₁₋₉alkylCO₂R⁹, —C₂₋₉alkenylCO₂R⁹, —C₂₋₉alkynylCO₂R⁹, -carbocyclyl-CO₂R⁹, or independently, an R⁷ and an R⁸ are taken together with the atoms to which they are attached to form a substituted or unsubstituted carbocyclyl or substituted or unsubstituted heterocyclyl, or independently a geminal R⁷ and R⁸ together form a C₂₋₉ alkenylenylCO₂R⁹;

each R⁹ is independently selected from a group consisting of H, —C₁₋₉alkyl, C₂₋₉alkenyl, —C₂₋₉alkynyl, carbocyclyl, —C₁₋₉alkylR¹¹, —C₂₋₉alkenylR¹¹, —C₂₋₉alkynylR¹¹, -carbocyclyl-R¹¹, substituted or unsubstituted aryl, substituted or unsubstituted heteroaryl, substituted or unsubstituted carbocyclyl, and substituted or unsubstituted heterocyclyl;

each R¹⁰ is independently selected from a group consisting of H, —C₁₋₉alkyl, —OR⁹, —CH(═NH), —C(═O)OR⁹, substituted or unsubstituted aryl, substituted or unsubstituted heteroaryl, substituted or unsubstituted carbocyclyl, and substituted or unsubstituted heterocyclyl;

each R¹¹ is independently selected from a group consisting of substituted or unsubstituted aryl, substituted or unsubstituted heteroaryl, substituted or unsubstituted carbocyclyl, and substituted or unsubstituted heterocyclyl;

X is selected from a group consisting of H, —CO₂R¹², and carboxylic acid isosteres;

R¹² is selected from a group consisting of H, C₁₋₉alkyl, —(CH₂)₀₋₃—R₁₁, —C(R¹³)₂OC(O)C₁₋₉alkyl, —C(R¹³)₂OC(O)R¹¹, —C(R¹³)₂OC(O)OC₁₋₉alkyl and —C(R¹³)₂OC(O)OR¹¹;

each R¹³ is independently selected from a group consisting of H and C₁₋₄alkyl; and

m is independently zero or an integer from 1 to 2,

wherein each C₁₋₉ alkyl, C₂₋₉ alkynyl, and C₂₋₉alkynyl is independently optionally substituted.

Some β-lactamase inhibitors useful with the methods, compositions and compounds provided herein are described in U.S. Provisional Application No. 61/529,859, incorporated herein by reference in its entirety. Some embodiments include a compound having the structure of formula II:

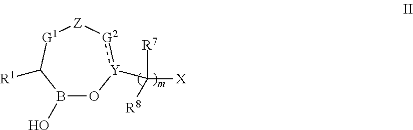

or pharmaceutically acceptable salt thereof, wherein:

R^{1α} is selected from a group consisting of —C₁₋₉ alkyl, —C₂₋₉ alkenyl, —C₂₋₉ alkynyl, —NR^{9α}R^{10α}, —C₁₋₉ alkylR^{11α}, —C₂₋₉ alkenylR^{11α}, —C₂₋₉ alkynylR^{11α}, -carbocyclyl-R^{11α}, —CH(OH)C₁₋₉alkylR^{9α}, —CH(OH)C₂₋₉alkenylR^{9α}, —CH(OH)C₂₋₉alkynylR^{9α}, —CH(OH)carbocyclyl-R^{9α}, —C(═O)R^{9α}, —C(═O)C₁₋₉alkylR^{9α}, —C(═O)C₂₋₉alkenylR^{9α}, —C(═O)C₂₋₉alkynylR^{9α}, —C(═O)C₂₋₉carbocyclyl-R^{9α}, —C(═O)NR^{9α}R^{10α}, —N(R^{9α})C(═O)R^{9α}, —N(R^{9α})C(═O)NR^{9α}R^{10α}, —N(R^{9α})C(═O)OR^{9α}, —N(R^{9α})C(═O)C(═NR^{10α})R^{9α}, —N(R^{9α})C(═O)C(═CR^{9α}R^{10α})R^{9α}, —N(R^{9α})C(═O)C₁₋₄alkylN(R^{9α})C(═O)R^{9α}, —N(R^{9α})C(═NR^{10α})R^{9α}, —C(═NR^{10α})NR^{9α}R^{10α}, —N═C(R^{9α})NR^{9α}R^{10α}, —N(R^{9α})SO₂R^{9α}, —N(R^{9α})SO₂NR^{9α}R^{10α}, —N═CHR^{9α}, —C(R^{9α}R^{10α})C(═O)NR^{9α}R^{10α}, —C(R^{9α}R^{10α})N(R^{9α})C(═O)R^{9α}, —C(R^{9α}R^{10α})OR^{9α}, substituted or unsubstituted aryl, substituted or unsubstituted heteroaryl, substituted or unsubstituted carbocyclyl, and substituted or unsubstituted heterocyclyl;

G^{1α} is selected from a divalent group consisting of —C(R^{aα}R^{bα})—, —C(═R^{a}′^{α})—, —C(R^{aα}R^{bα})C(R^{cα}R^{dα})—, —C(R^{aα})═C(R^{cα})—, —C(═O)C(R^{aα}R^{bα})—, —C(R^{aα}R^{bα})C(═O)—, and a bond;

G^{2α} is selected from a divalent group consisting of —C(R^{eα}R^{fα})—, —C(═R^{e}′^{α})—, ═C(R^{eα})—, —C(R^{eα}R^{fα})C(R^{gα}R^{hα})—, —C(R^{eα}R^{fα})C(R^{gα}R^{hα})C(R^{iα}R^{jα})—, —C(═O)—, —C(═O)C(R^{eα}R^{fα})—, —C(R^{eα}R^{fα})C(═O)—, —C(═O)C(R^{eα}R^{fα})C(R^{gα}R^{hα})—, —C(R^{eα}R^{fα})C(R^{gα}R^{hα})C(═O)—, —C(═O)C(R^{eα}R^{fα})C(R^{gα}R^{hα})C(R^{iα}R^{Jα})—, —C(R^{eα}R^{fα})C(R^{gα}R^{hα})C(R^{iα}R^{Jα})C(═O)—, —C(R^{eα})═C(R^{gα})—, —C(R^{eα})═C(R^{gα})C(R^{iα}R^{Jα})— and —C(R^{eα}R^{fα})C(R^{gα})═C(R^{Jα})—;

R^{aα}, R^{bα}, R^{cα}, R^{dα}, R^{eα}, R^{fα}, R^{gα}, R^{hα}, R^{iα}, and R^{Jα} are independently selected from a group consisting of H, Cl, F, CN, CF₃, —R^{9α}, —OR^{9α}, NR^{9α}R^{10α}, —C(═O)NR^{9α}R^{10α}, and —C(═O)OR^{9α}, or independently: R^{aα} and R^{cα}, R^{eα} and an R^{7α}, R^{kα} and R^{cα}, R^{kα} and R^{eα}, R^{eα} and R^{gα}, and R^{gα} and R^{jα} are taken together with the atoms to which they are attached and any intervening atoms to form a substituted or unsubstituted aryl, substituted or unsubstituted heteroaryl, substituted or unsubstituted carbocyclyl or substituted or unsubstituted heterocyclyl, or independently R^{eα} and R^{fα} are taken together with the atoms to which they are attached and any intervening atoms to form a substituted or unsubstituted carbocyclyl or substituted or unsubstituted heterocyclyl;

R^{a}′^{α} and R^{e}′^{α} are ═CR^{9α}R^{10α} or independently R^{a}′^{α} and R^{kα}, or R^{e}′^{α} and R^{kα}, are taken together with the atoms to which they are attached to form a substituted or unsubstituted aryl, substituted or unsubstituted heteroaryl, substituted or unsubstituted carbocyclyl, or substituted or unsubstituted heterocyclyl;

Z^{α} is selected from a divalent group consisting of —C(R^{9α}R^{10α})—, —O—, —S—, —N(R^{9α})—, —N[C(═O)R^{9α}]—, —N[C(═O)NR^{9α}R^{10a}]—, —N[C(═O)OR^{9α}]—, —N[C(═NR^{10α})R^{9α}]—, —N[SO₂R^{9α}]—, —N[SO₂NR^{9α}R^{10α}]—, —N(R^{9α})C(═O)—, —C(R^{9α}R^{kα})—, —C(═R^{kα})—, —N(R^{kα})—, and a bond;

R^{kα} and R^{cα}, R^{kα} and R^{eα}, R^{a}′^{α} and R^{kα}, or R^{e}′^{α} and R^{kα} are taken together with any intervening atoms to form a substituted or unsubstituted aryl, substituted or unsubstituted heteroaryl, substituted or unsubstituted carbocyclyl or substituted or unsubstituted heterocyclyl;

Y^{α} is selected from a group consisting of N, CR^{6α}, and C, with the proviso that when Z^{α} is a bond, —C(R^{9α}R^{10α})—, —C(R^{9α}R^{kα})—, or —C(═R^{kα})—, then Y^{α} is N;

R^{6α} is selected from a group consisting of H, —C₁₋₉alkyl, —C₂₋₉alkenyl, —C₂₋₉alkynyl, carbocyclyl, —C₂₋₉alkenylR^{11α}, —C₂₋₉alkynylR^{11α}, carbocyclyl-R^{11α}, —C(═O)OR^{9α} and —C₁₋₉alkylCO₂R^{9α}, —C₂₋₉alkenylCO₂R^{9α}, C₂₋₉alkynylCO₂R^{9α}, and -carbocyclyl-CO₂R^{9α}, or alternatively R^{6α} and an R^{7α} or R^{6α} and R^{eα} are taken together with the atoms to which they are attached and any intervening atoms to form a substituted or unsubstituted carbocyclyl or substituted or unsubstituted heterocyclyl;

each R^{7α} is independently selected from a group consisting of H, halo, —C₁₋₉alkyl, —C₂₋₉alkenyl, —C₂₋₉alkynyl, —NR^{9α}R^{10α}, —OR^{9α}, —C₁₋₉alkylCO₂^{9α}, —C₂₋₉alkenylCO₂R^{9α}, —C₂₋₉alkynylCO₂R^{9α}, and -carbocyclyl-CO₂R^{9α}, or independently, R^{6α} and an R^{7α} or an R^{7α} and an R^{8α} are taken together with the atoms to which they are attached and any intervening atoms to form a substituted or unsubstituted carbocyclyl or substituted or unsubstituted heterocyclyl, or independently an R^{7a} and R^{ea} are are taken together with intervening atoms to form a substituted or unsubstituted aryl, substituted or unsubstituted heteroaryl, substituted or unsubstituted carbocyclyl, or substituted or unsubstituted heterocyclyl;

each R^{8α} is independently selected from a group consisting of H, halo, —C₁₋₉alkyl, —C₂₋₉alkenyl, —C₂₋₉alkynyl, —NR^{9α}R^{10α}, OR^{9α}, —C₁₋₉alkylCO₂R^{9α}, —C₁₋₉alkylCO₂R^{9α}, —C₂₋₉alkenylCO₂R^{9α}, —C₂₋₉alkynylCO₂R^{9α}, and -carbocyclyl-CO₂R^{9α}, or independently, and R^{7α} and an R^{8α} are taken together with the atoms to which they are attached to form a substituted or unsubstituted carbocyclyl or substituted or unsubstituted heterocyclyl, or independently, each R^{8α} attached to a ring atom forming part of the substituted or unsubstituted aryl or a substituted or unsubstituted heteroaryl is absent;

each R^{9α} is independently selected from a group consisting of H, —C₁₋₉alkyl, C₂₋₉alkenyl, —C₂₋₉alkynyl, carbocyclyl, —C₁₋₉alkylR^{11α}, C₂₋₉alkenylR^{11α}, —C₂₋₉alkynylR^{11α}, -carbocyclyl-R^{11α}, —C₁₋₉alkylCO₂R^{12α}, C₂₋₉alkenylCO₂R^{12α}, C₂₋₉alkynylCO₂R^{12α}, -carbocyclyl-CO₂R^{12α}, —C₁₋₉alkyl-N(R^{12α})OR^{12α}, C₂₋₉alkenyl-N(R^{12α})OR^{12α}, —C₂₋₉alkynyl-N(R^{12α})OR^{12α}, -carbocyclyl-N(R^{12α})OR^{12α}, —C₁₋₉alkyl-OR^{12α}, C₂₋₉alkenyl-OR^{12α}, —C₂₋₉alkynyl-OR^{12α}, -carbocyclyl-OR^{12α}, substituted or unsubstituted aryl, substituted or unsubstituted heteroaryl, substituted or unsubstituted carbocyclyl, and substituted or unsubstituted heterocyclyl;

each R^{10α} is independently selected from a group consisting of H, —C₁₋₉alkyl, —OR^{9α}, —CH(═NH)—, —C(═O)OR^{9α}, substituted or unsubstituted aryl, substituted or unsubstituted heteroaryl, substituted or unsubstituted carbocyclyl, and substituted or unsubstituted heterocyclyl;

each R^{11α} is independently selected from a group consisting of substituted or unsubstituted aryl, substituted or unsubstituted heteroaryl, substituted or unsubstituted carbocyclyl, and substituted or unsubstituted heterocyclyl;

each R^{12α} is independently selected from a group consisting of H, C₁₋₉alkyl, —(CH₂)₀₋₃—R^{11α}, —C(R^{13α})₂OC(O)C₁₋₉alkyl, —C(R^{13α})₂OC(O)R^{11α}, —C(R^{13α})₂OC(O)OC₁₋₉alkyl and —C(R^{13α})₂OC(O)OR^{11α};

each R^{13α} is independently selected from a group consisting of H and C₁₋₄alkyl;

each X^{α} is independently selected from a group consisting of H, —CO₂R^{12α}, and carboxylic acid isosteres;

m^{α} is independently zero or an integer from 1 to 2;

the bond represented by a dashed and solid line represents a bond selected from the group consisting of a single bond and a double bond; and

each C₁₋₉alkyl, C₂₋₉alkenyl, and C₂₋₉alkynyl is optionally substituted.

Some β-lactamase inhibitors useful with the methods, compositions and compounds provided herein are described in U.S. Pub. No. 2010/0120715, incorporated herein by reference in its entirety. Some embodiments include a compound having the following structure:

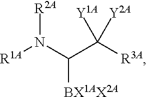

wherein, R^{1A} is —C(O)R^{4A}; —C(O)NR^{4A}R^{5A}; —C(O)OR⁴; —S(O)₂R^{4A}, —C(═NR^{4A}R^{5A})R^{4A}, C(═NR^{4A}R^{5A})NR^{4A}R^{5A}, hydrogen, or is selected from the group consisting of: (a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (c) heterocyclic group substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;

R^{2A} is hydrogen, or is selected from the group consisting of: (a) C₁-C₆ alkyl any carbon of which can be substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₆ carbons comprise part of said oxyimino group, sulfido, and sulfoxido, (b) C₃-C₇ cycloalkyl any carbon of which can be substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

R^{3A} is an aryl or heteroaryl group substituted with from 1 to 4 substituents wherein one of the substituents is a hydroxyl or amino group located at the 2 position relative to the group containing Y^{1A} and Y^{2A}, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;

R^{4A} is selected from the group consisting of: (a) C₁-C₁₀ alkyl any carbon of which can be substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₁₀ carbons comprise part of said oxyimino group, sulfido, and sulfoxido, (b) C₃-C₁₀ cycloalkyl any carbon of which can be substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

R^{5A} is hydrogen or is selected from the group consisting of: (a) C₁-C₆ alkyl any carbon of which can be substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₁₀ carbons comprise part of said oxyimino group, sulfido, and sulfoxido, (b) C₃-C₇ cycloalkyl any carbon of which can be substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxy 1, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, oxo, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

X^{1A} and X^{2A} are independently hydroxyl, halogen, NR^{4A}R^{5A}, C₁-C₆ alkoxy, or when taken together X^{1A} and X^{2A} form a cyclic boron ester where said chain or ring contains from 2 to 20 carbon atoms and, optionally, 1-3 heteroatoms which can be O, N, or S, or when taken together X^{1A} and X^{2A} form a cyclic boron amide where said chain or ring contains from 2 to 20 carbon atoms and, optionally, 1-3 heteroatoms which can be O, N, or S, or when taken together X^{1A} and X^{2A} form a cyclic boron amide-ester where said chain contains from 2-20 carbon atoms and, optionally, 1-3 heteroatoms which can be O, N, or S, or X^{1A} and R^{1A} together form a cyclic ring where said ring contains 2 to 10 carbon atoms and, optionally, 1-3 heteroatoms which can be O, N, or S, and X^{2A} is hydroxyl, halogen, NR^{4A}R^{5A}, C₁-C₆ alkoxy, or X^{1A} and R^{3A} together form a cyclic ring where said ring contains 3 to 10 carbon atoms and, optionally, 1-3 heteroatoms which can be O, N, or S, and X^{2A} is hydroxyl, halogen, NR^{4A}R^{5A}, or C₁-C₆ alkoxy;

Y^{1A} and Y^{2A} are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y^{1A} and Y^{2A} form a cyclic structure containing from 3-12 carbon atoms and, optionally, 1-3 heteroatoms which can be O, N, or S;

or a salt thereof;

provided that, when R^{1A} is —C(O)R^{4A}, R^{2A} is hydrogen, R^{3A} is a phenyl group having two substituents consisting of a hydroxyl at the 2-position and a carboxylic acid at the 3-position relative to the group containing Y^{1A} and Y^{2A}, X^{1A} and X^{2A} are hydroxyl or X^{1A} is hydroxyl and X^{2A} is replaced by the ortho-hydroxyl oxygen of R^{3A} such that a 6-membered ring is formed, and Y^{1A} and Y^{2A} are hydrogen, R^{4A} is not unsubstituted C₁ alkyl.

Some β-lactamase inhibitors useful with the methods, compositions and compounds provided herein are described in U.S. Pat. No. 6,184,363, incorporated herein by reference in its entirety. Some embodiments include compounds having the following formula:

(OH)₂—B—R^{B}

wherein, R^{B} is naphthalene, phenanthrene, or has one of the following formulas:

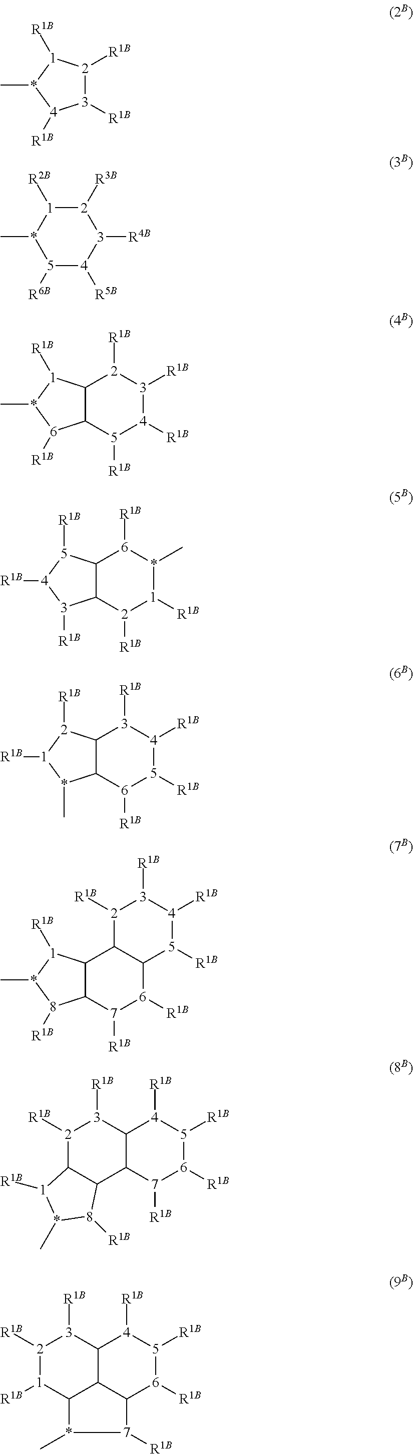

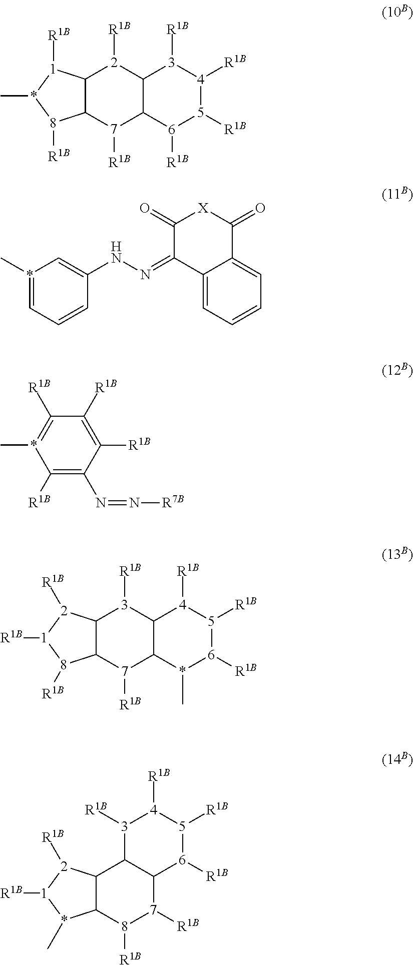

wherein, ring system (2^{B}), (3^{B}) (4^{B}) (5^{B}) (6^{B}) (7^{B}) (8^{B}) (9^{B}), (10^{B}), (13^{B}) or (14^{B}) is aromatic or nonaromatic;

the atom center * is (R) or (S) in the case of chiral compounds; positions 1, 2, 3, 4, 5, 6, 7 and 8 each independently is C, N, O or S;

R^{1B} through R^{6B} each independently is a lone pair, H, B(OH)₂, a halogen atom, CF₃, CH₂ CF₃, CCl₃, CH₂ CCl₃, CBR^{3B}, CH₂ CBR^{3B}, NO₂, lower alkyl, CO₂H, CHCHCOOH, CH₂CH₂CH₂ COOH, SO₃H, PO₃H, OSO₃H, OPO₃H, OH, NH₂, CONH₂, COCH₃, OCH₃, or phenyl boronic acid, except that R^{2B}, R^{3B}, R^{4B}, R^{5B} and R^{6B} cannot all simultaneously be H, R^{2B} cannot be lower alkyl when R^{3B}, R^{4B}, R^{5B} and R^{6B} are H, R^{3B} cannot be NH₂, OH or lower alkyl when R^{2B}, R^{4B}, R^{5B} and R^{6B} are H, and R^{4B} cannot be lower alkyl when R^{2B}, R^{3B}, R^{5B} and R^{6B} are H;

R^{7B} is H, CF₃, CCl₃, CBR^{3B}, CH₂CF₃, CH₂CCl₃, CH₂CBR^{3B}, NO₂, COCH₃, OCH₃, lower alkyl, cyclic alkene, cyclic alkene substituted with one or more substituents R^{8B}, heterocyclic alkene, or heterocyclic alkene substituted with one or more substituents R^{8B};

each R^{8B} is independently H, B(OH)₂, a halogen atom, CF₃, CCl₃, CBR^{3B}, CH₂CF₃, CH₂CCl₃, CH₂CBR^{3B}, NO₂, lower alkyl, OH, NH₂, N(CH₃)₂, N(CH₃)CH₂CH₃, NHCOCH₃, COOH, CHCHCOOH, CH₂CH₂CH₂COOH, COCH₃, OCH₃, phenyl boronic acid, CONH₂, CONHCH₂COOH, CONHCH₂CONH₂, CONHCH₂CONHCH₂R^{10B}, SO₂NH₂, SO₂NHCH₂COOH, SO₂NHCH₂CONH₂, or SO₂NHCH₂CONHCH₂R^{10B};

X is O, NH, NCH₃ or

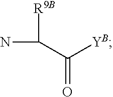

Y^{B} is OH, NH₂, NCH₃, N(CH₃)₂, NHCOCH₃ or NHCOCH₂COOH;

R^{9B} is H, a halogen atom, CF₃, CCl₃, CBR^{3B}, CH₂ CF₃, CH₂ CCl₃, CH₂CBR^{3B}, NO₂, CO₂H, CHCHCOOH, CH₂CH₂CH₂COOH, SO₃H, PO₃H, OSO₃H, OPO₃H, OH, NH₂, CONH₂, COCH₃, OCH₃, phenyl boronic acid, lower alkyl, or a side chain of a standard amino acid; and

R^{10B} is a side chain of a standard amino acid.

Some β-lactamase inhibitors useful with the methods, compositions and compounds provided herein are described in U.S. Pub. No. 2010/0256092, incorporated herein by reference in its entirety. Some embodiments include compounds having the following formula:

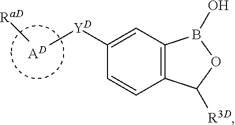

wherein, A^{D} is a member selected from cycloalkyl, heterocycloalkyl, aryl and heteroaryl;

Y^{D} is a member selected from O and —S(O)₂NH— wherein the sulfur in —S(O)₂NH— is covalently attached to A^{D};

R^{3D} is a member selected from H, cyano and substituted alkyl;

R^{aD} is a member selected from H, —OR¹⁰D, —NR^{10D}R^{11D}, —SR^{10D}, —S(O)R^{10D}, —S(O)₂R¹⁰D, —S(O)₂NR^{10D}R^{11D}, —C(O)R^{10D}, —C(O)OR^{10D}, —C(O)NR^{10D}R^{11D}, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl,

wherein, each R^{10D} and each R^{11D} is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl,

with the proviso that R^{10D} and R^{11D}, together with the nitrogen to which they are attached, are optionally combined to form a 5- to 7-membered substituted or unsubstituted heterocycloalkyl ring;

with the proviso that when Y^{D} is O, R^{3D} is a member selected from cyano and substituted alkyl; with the proviso that when Y^{D} is —S(O)₂NH—, R^{3D} is H, and R^{aD} is not H or unsubstituted alkyl or halosubstituted alkyl,

and salts thereof.

Some β-lactamase inhibitors useful with the methods, compositions and compounds provided herein are described in U.S. Pat. No. 7,271,186, incorporated herein by reference in its entirety. Some embodiments include compounds having the following formula:

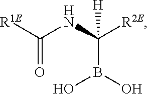

wherein, R^{1E} is a substituent selected from hydrogen, alkyl, alkenyl, cycloalkenyl, and heterocyclyl moieties, providing R^{1E} is not methyl and R^{1E} is not phenyl; and wherein R^{2E} is a substituent selected from heterocyclyl, cycloalkenyl, alkenyl and alkyl moieties.

Some β-lactamase inhibitors useful with the methods, compositions and compounds provided herein are described in U.S. Pub. No. 2011/0288063, incorporated herein by reference in its entirety. Some embodiments include compounds having the following formula:

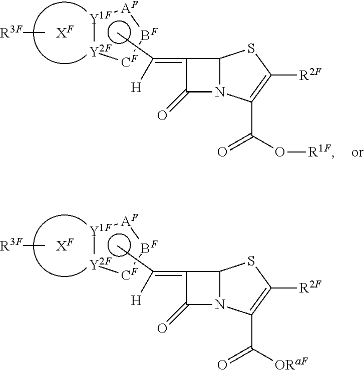

wherein, R^{1F} is the residue of a carboxy protecting group;

R^{aF} is hydrogen or a pharmaceutically-acceptable salt forming agent or a pharmaceutically-acceptable ester residue readily hydrolyzable in vivo;

R^{2F} is selected from the group consisting of: (a) Hydrogen, (b) straight or branched chain alkyl, (c) hydroxymethyl, (d) alkoxymethyl, (e) aminocarbonyloxymethyl, (f) aryl, (g) heteroaryl and (h) heterocyclyl;

heteroaryl means a 5- or 6-membered unsaturated aromatic ring containing from 1 to 4 of any one or more of the hetero atoms selected from O, S and N; heterocyclyl means a 5-membered saturated ring containing one hetero atom;

X^{F} is a bridged bicyclic ring system having optionally one or two hetero atoms selected from O, S and N; the ring X^{F} may be optionally substituted with R^{3F} wherein

R^{3F} is selected from (a) hydrogen, (b) alkyl, (c) hydroxy, (d) alkoxy, (e) hydroxymethyl, (f) alkoxymethyl, (g) halogen, (h) cyano, (i) carboxy, (j) alkoxycarbonyl, (k) amino, (l) aminoalkyl, (m) mono- or diallylamino, (n) mono- or dialkylaminoalkyl, (o) acylamino, (p) sulfonylamino, (q) substituted or unsubstituted amidino, (r) substituted or unsubstituted urea, (s) substituted or unsubstituted thiourea, (t) substituted or unsubstituted carboxamido, (u) substituted or unsubstituted thiocarboxamido, (v) substituted or unsubstituted aryl, (w) substituted or unsubstituted aralkyl, (x) substituted or unsubstituted heteroaryl, (y) substituted or unsubstituted heteroarylalkyl and (z) substituted or unsubstituted heterocyclylalkyl;

the heteroaryl groups mentioned in items (x) and (y) means a 5- or 6-membered unsaturated aromatic ring containing from 1 to 4 of any one or more of the hetero atoms selected from O, S and N, wherein the said heteroaryl groups could be bonded via carbon, or a nitrogen-containing heteroaryl group could be bonded via nitrogen;

the bridged bicyclic ring systems containing a NH ring atom may be optionally substituted on the said nitrogen by a substituent selected from: (a) alkyl, (b) alkenyl, (c) alkynyl, (d) cycloalkyl, (e) cycloalkylalkyl, (f) cycloalkenyl, (g) cycloalkenylalkyl, (h) aryl, (i) arylalkyl, (j) heteroaryl, (k) heteroarylalkyl, (l) heterocyclyl, (m) heterocyclylalkyl (n) or a protecting group;

Y^{1F} and Y^{2F} may independently be C or N;

A^{F}, B^{F} or C^{F} form part of a heteroaryl ring where one of A^{F}, B^{F} or C^{F} is a carbon atom to which the remainder of the molecule is attached, and A^{F} B^{F} and C^{F} are independently selected from CR^{4F}, O, N, S or NR^{5F};

R^{4F} is hydrogen; and

R^{5F} is selected from the group consisting of: (a) hydrogen, (b) straight or branched lower alkyl, (c) lower alkenyl, (d) lower alkynyl, (e) hydroxy alkyl, (f) alkoxy alkyl, (g) aminocarbonyloxy alkyl, (h) cyano alkyl, (i) aminoalkyl, (j) mono- or dialkylaminoalkyl, (k) alkoxycarbonylalkyl, (l) carboxyalkyl, (m) substituted or unsubstituted carboxamidoalkyl, (n) cycloalkylalkyl, (o) substituted or unsubstituted thiocarboxamidoalkyl, (p) substituted or unsubstituted amidinoalkyl, (q) substituted or unsubstituted guanidinoalkyl, (r) substituted or unsubstituted aminocarbonylaminoalkyl, (s) acylaminoalkyl, (t) aralkyl, (u) heteroarylalkyl and (v) heterocyclylalkyl.

Some β-lactamase inhibitors useful with the methods, compositions and compounds provided herein are described in U.S. Pub. No. 2005/0020572, incorporated herein by reference in its entirety. Some embodiments include compounds having the following formula:

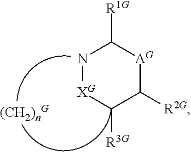

wherein, R^{1G} is hydrogen, COOH, CN, COOR^{G}, CONR^{6G}R^{7G}, (CH₂)ₙ^{G}R^{5G} or C(═NR^{6G})NHR^{7G};

R^{G} is selected from the group consisting of alkyl containing 1 to 6 carbon atoms optionally substituted by a pyridyl or carbamoyl radical, —CH₂-alkenyl containing 3 to 9 carbon atoms, aryl containing 6 to 10 carbon atoms and aralkyl containing 7 to 11 carbon atoms, wherein the nucleus of said aryl or aralkyl is optionally substituted by OH, NH₂, NO₂, alkyl containing 1 to 6 carbon atoms, alkoxy containing 1 to 6 carbon atoms or by one or more halogen atoms;

R^{6G} and R^{7G} are identical or different and are independently selected from the group consisting of hydrogen, alkyl containing 1 to 6 carbon atoms, aryl containing 6 to 10 carbon atoms and aralkyl containing 7 to 11 carbon atoms optionally substituted by a carbamoyl, ureido or dimethylamino radical, and alkyl containing 1 to 6 carbon atoms substituted by a pyridyl radical;

n^{1G} is 1 or 2;

R^{5G} is selected from the group consisting of COOH, CN, OH, NH₂, CO—NR^{6G}R^{7G}, COOR^{G}, OR^{G}, OCHO, OCOR^{G}, OCOOR^{G}, OCONHR^{G}, OCONH₂, NHR^{G}, NHCOH, NHCOR^{G}, NHSO₂R^{G}, NH—COOR^{G}, NH—CO—NHR^{G} and NHCONH₂, wherein R^{G}, R^{6G} and R^{7G} are as defined above;

R^{2G} is hydrogen or (CH₂)ₙ^{1G}₁R^{5G} wherein n^{1G} is 0, 1 or 2, and

R^{5G} is as defined above;

R^{3G} is hydrogen or alkyl containing 1 to 6 carbon atoms;

A^{G} is a bond between the two carbons which carry R^{1G} and R^{2G},

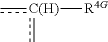

group wherein R^{4G} is hydrogen or (CH₂)ₙ^{1G}₁R^{5G} and n^{1G} and R^{5G} are as defined above, and the dotted line is an optional bond with one of the two carbons which carry R^{1G} and R^{2G};

n^{G} is 1 or 2;

X^{G} is a divalent —C(O)—B^{G}— group linked to the nitrogen atom by the carbon atom wherein B^{G} is a divalent —O—(CH₂)ₙ^{2G}— group linked to the carbonyl by the oxygen atom, a divalent —NR^{8G}—(CH₂)ₙ^{2G}— or —NR^{8G}—O— group linked to the carbonyl by the nitrogen atom, n^{2G} is 0 or 1, and wherein B^{G} is —NR^{8G}—(CH₂)ₙ^{2G}—, R^{8G} is selected from the group consisting of hydrogen, OH, R^{G}, OR^{G}, Y^{G}, OY^{G}, Y^{1G}, OY^{1G}, Y^{2G}, OY^{2G}, Y^{3G}, OCH₃CH₂SOₘ^{G}R^{G}, OSiR^{aG}R^{bG}R^{cG} and SiR^{aG}R^{bG}R^{cG} and wherein B^{G} is —NR^{aG}—O—, R^{8G} is selected from the group consisting of hydrogen, R, Y^{G}, Y^{1G}, Y^{2G}, Y^{3G} and SiR^{aG}R^{bG}R^{cG}, wherein R^{aG}, R^{bG} and R^{cG} is each independently a linear or branched alkyl containing 1 to 6 carbon atoms or aryl containing 6 to 10 carbon atoms, R^{G} is as defined above and m^{G} is 0, 1 or 2;

Y^{G} is selected from the group consisting of COH, COR^{G}, COOR^{G}, CONH₂, CONHR^{G}, CONHOH, CONHSO₂R^{G}, CH₂COOH, CH₂COOR^{G}, CH₂CONHOH, CH₂CONHCN, CH₂tetrazole, protected CH₂tetrazole, CH₂SO₃H, CH₂SO₂R^{G}, CH₂PO(OR^{G})₂, CH₂PO(OR^{G})(OH), CH₂PO(R^{G})(OH) and CH₂PO(OH)₂;

Y^{1G} is selected from the group consisting of SO₂R^{G}, SO₂NHCOH, SO₂NHCOR^{G}, SO₂NHCOOR^{G}, SO₂NHCONHR^{G}, SO₂NHCONH₂ and SO₃H;

Y^{2G} is selected from the group consisting of PO(OH)₂, PO(OR^{G})₂, PO(OH)(OR^{G}) and PO(OH)(R^{G});

Y^{3G} is selected from the group consisting of tetrazole, tetrazole substituted by R^{G}, squarate, NH or NR^{G}-tetrazole, NH or NR^{G}-tetrazole substituted by R^{G}, NHSO₂R^{G} and NR^{G}SO₂R^{G} wherein R^{G} is as defined above; and

R^{1G}, R^{2G} and R^{3G} are not simultaneously hydrogen when n^{G} is 1, A^{G} is

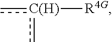

wherein R^{4G} is hydrogen and

X^{G} is —C(O)—O—(CH₂)ₙ^{G2} wherein n^{G2} is 0 or 1, or

X^{G} is —CO—NR^{8G}—(CH₂)ₙ^{G2} wherein n^{G2} is 1 and R^{8G} is isopropyl, or

X^{G} is —CO—NR^{8G}—(CH₂)ₙ^{G2} wherein n^{G2} is 0 and R^{8G} is hydrogen or phenyl.

Some β-lactamase inhibitors useful with the methods, compositions and compounds provided herein include the compound NX1.104, having the following formula:

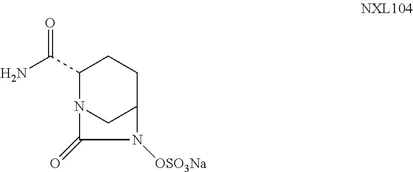

Some β-lactamase inhibitors useful with the methods, compositions and compounds provided herein are described in U.S. Pub. No. 2004/0157826, incorporated herein by reference in its entirety. Some embodiments include compounds having the following formula:

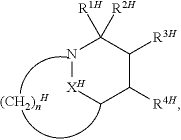

wherein, either:

a) R^{1H} is a radical selected from the group consisting of hydrogen, COOH, COOR, CN, (CH₂)ₙ^{1H}R^{5H}, CONR^{6H}R^{7H} and

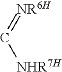

R^{H} is selected from the group consisting of an alkyl radical containing from 1 to 6 carbon atoms, optionally substituted with one or more halogen atoms or with a pyridyl radical; a —CH₂— alkenyl radical containing in total from 3 to 9 carbon atoms; a (poly)alkoxyalkyl group containing 1 to 4 oxygen atoms and 3 to 10 carbon atoms; an aryl radical containing from 6 to 10 carbon atoms or an aralkyl radical containing from 7 to 11 carbon atoms, the nucleus of the aryl or aralkyl radical being optionally substituted with a radical selected from the group consisting of OH, NH₂, NO₂, alkyl containing from 1 to 6 carbon atoms, alkoxy containing from 1 to 6 carbon atoms and one or more halogen atoms;

R^{5H} is selected from the group consisting of COOH, CN, OH, NH₂, CO—N,

R^{6H}R^{7H}, COOR^{H} and OR^{H} radicals, R^{H} being as defined above, R^{6H} and R^{7H} are individually selected from the group consisting of hydrogen, an alkyl radical containing from 1 to 6 carbon atoms, an alkoxy radical containing from 1 to 6 carbon atoms, an aryl radical containing from 6 to 10 carbon atoms, an aralkyl radical containing from 7 to 11 carbon atoms and an alkyl radical containing from 1 to 6 carbon atoms which is substituted with a pyridyl radical;

n^{1H} is equal to 1 or 2,

R^{3H} and R^{4H}, together with the carbons to which they are attached, form a phenyl or a 5- or 6-membered aromatic heterocycle containing from 1 to 4 heteroatoms selected from nitrogen, oxygen and sulfur, which is substituted with one or more R¹H groups, R^{1H} being a radical selected from the group consisting of: —(O)ₐ^{H}—(CH₂)_{b}^{H}—(O)ₐ^{H}—CONR^{6H}R^{7H}, —(O)ₐ^{H}—(CH₂)_{b}^{H}—OSOₐ^{H}, —(O)ₐ^{H}—(CH₂)_{b}^{H}—SO₃, —(O)ₐ^{H}—SO₂R^{H}, —(O)ₐ^{H}—SO₂—CHa^{H}I₃, —(O)ₐ^{H}—(CH₂)_{b}^{H}—NR^{6H}R^{7H}, — (O)ₐ^{H}—(CH₂)_{b}^{H}—NH—COOR^{H}, —(CH₂)_{b}^{H}—COOH, —(CH₂)_{b}^{H}—COOR^{H}, —OR^{H}″, OH, —(CH₂)_{b}^{H}— phenyl, and —(CH₂)_{b}^{H}-5- or 6-membered aromatic heterocycle containing from 1 to 4 heteroatoms selected from nitrogen, oxygen and sulfur,

each of said phenyl and said heterocycle being optionally substituted with one or more substituents selected from halogen, alkyl containing from 1 to 6 carbon atoms, alkoxy containing from 1 to 6 carbon atoms and CF₃,

R^{H}, R^{6H} and R^{7H} being as defined above,

R^{H}″ being selected from alkyl radicals containing from 1 to 6 carbon atoms substituted with one or more radicals selected from hydroxy, protected hydroxy, oxo, halogen and cyano radicals,

a^{H} being equal to 0 or 1 and b being an integer from 0 to 6,

provided that, when R^{1H} is OH, R^{1H} is CONR^{6H}R^{7H} in which one of R^{6H} and R^{7H} is an alkoxy containing from 1 to 6 carbon atoms; or

b) R^{4H} is hydrogen or (CH₂)ₙ^{1H}₁ R^{5H}, wherein n^{1H}₁, is 0, 1 or 2 and R^{5H} is as defined above,

and R^{1H} and R^{3H}, together with the carbons to which they are attached, form a substituted phenyl or heterocycle, as defined above;

and, in both cases a) and b),

R^{2H} is selected from the group consisting of hydrogen, halogen, R^{H}, S(O)ₘ^{H}R^{H}, OR^{H}, NHCOR^{H}, NHCOOR^{H} and NHSO₂R^{H}, R being as defined above and m^{H} being 0, 1 or 2,

X^{H} is a divalent group —C(O)—B^{H}— linked to the nitrogen atom by the carbon atom,

B^{H} is a divalent group selected from 1) —O—(CH₂)ₙ″^{H}— linked to the carbonyl by the oxygen atom, 2) —NR^{8H}—(CH₂)ₙ″^{H}— and 3) —NR^{8H}—O— linked to the carbonyl by the nitrogen atom, n″^{H} is 0 or 1 and R^{8H} is a radical selected from the group consisting of hydrogen, OH, R^{H}, OR^{H}, Y^{H}, OY^{H}, Y^{1H}, OY^{1H}, Y^{2H}, OY^{2H}, Y^{3H}, O—CH₂—CH₂—S(O—)ₘ^{H}—R^{H}, SiR^{aH}R^{bH}R^{cH} and OSiR^{aH}R^{bH}R^{cH} wherein each of R^{aH}, R^{bH} and R^{cH} is a linear or branched alkyl containing from 1 to 6 carbon atoms or an aryl containing from 6 to 10 carbon atoms, and R^{H} and m^{H} are as defined above;

Y^{H} is selected from the group consisting of COH, COR^{H}, COOR^{H}, CONH₂, CONHR^{H}, CONHOH, CONHSO₂R^{H}, CH₂COOH, CH₂COOR^{H}, CHF—COOH, CHF—COOR^{H}, CF₂—COOH, CF₂—COOR^{H}, CN, CH₂CN, CH₂CONHOH, CH₂CONHCN, CH₂tetrazole, protected CH₂tetrazole, CH₂SO^{3H}, CH₂SO₂R^{H}, CH₂PO(OR^{H})₂, CH₂PO(OR^{H})(OH), CH₂PO(R^{H})(OH) and CH₂PO(OH)₂;

Y^{1H} is selected from the group consisting of SO₂R^{H}, SO₂NHCOH, SO₂NHCOR^{H}, SO₂NHCOOR^{H}, SO₂NHCONHR^{H}, SO₂NHCONH₂ and SO^{3H};

Y^{2H} is selected from the group consisting of PO(OH)₂, PO(OR^{H})₂, PO(OH)(OR^{H}) and PO(OH)(R^{H});

Y^{3H} is selected from the group consisting of tetrazole, tetrazole substituted with R^{H}, squarate, NH or NR^{H}tetrazole, NH or NR^{H}tetrazole substituted with R^{H}, NHSO₂R^{H}, NR^{H}SO₂R^{H}, CH₂tetrazole and CH₂tetrazole substituted with R^{H}, R^{H} being as defined above, and

n^{H} is 1 or 2, or one of its salts with a base or an acid.

Some β-lactamase inhibitors useful with the methods, compositions and compounds provided herein are described in U.S. Pat. No. 7,439,253 incorporated herein by reference in its entirety. Some embodiments include compounds having the following formula:

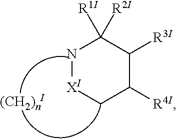

wherein, either:

a) R^{1I} is a radical selected from the group consisting of hydrogen, COOH, COOR^{I}, CN, (CH₂)ₙ′^{I}R^{5I}, CONR^{6I}R^{7I} and

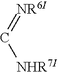

R^{I} is selected from the group consisting of an alkyl radical containing from 1 to 6 carbon atoms, optionally substituted with one or more halogen atoms or with a pyridyl radical; a —CH₂— alkenyl radical containing in total from 3 to 9 carbon atoms; a (poly)alkoxyalkyl group containing 1 to 4 oxygen atoms and 3 to 10 carbon atoms; an aryl radical containing from 6 to 10 carbon atoms or an aralkyl radical containing from 7 to 11 carbon atoms, the aryl or aralkyl radical being optionally substituted with a radical selected from the group consisting of OH, NH₂, NO₂, alkyl containing from 1 to 6 carbon atoms, alkoxy containing from 1 to 6 carbon atoms and one or more halogen atoms;

R^{5I} is selected from the group consisting of COOH, CN, OH, NH₂, CO—NR^{6I}R^{7I}, COOR^{I} and OR^{I} radicals, R^{I} being as defined above,

R^{6I} and R^{7I} are individually selected from the group consisting of hydrogen, an alkyl radical containing from 1 to 6 carbon atoms, an alkoxy radical containing from 1 to 6 carbon atoms, an aryl radical containing from 6 to 10 carbon atoms, an aralkyl radical containing from 7 to 11 carbon atoms and an alkyl radical containing from 1 to 6 carbon atoms which is substituted with a pyridyl radical;

n′^{I} is equal to 1 or 2,

R^{3I} and R^{4I}, together with the carbons to which they are attached, form a phenyl or a 5- or 6-membered aromatic heterocycle containing from 1 to 4 heteroatoms selected from nitrogen, oxygen and sulfur, which is substituted with one or more R′^{I} groups, R′^{I} being a radical selected from the group consisting of:

—(O)ₐ^{I}—(CH₂)_{b}^{I}—(O)ₐ^{I}CONR^{6I}R^{7I}, —(O)ₐ^{I}—(CH₂)_{b}—OSO₃H, —(O)ₐ^{I}—(CH₂)_{b}^{I}—SO₃H, —(O)ₐ^{I}—SO₂R^{I}, —(O)ₐ^{I}—SO₂—CHₐ^{I}I₃, —(O)ₐ^{I}—(CH₂)_{b}^{I}—NR^{6I}R^{7I}, —(O)ₐ^{I}—(CH₂)_{b}^{I}—NH—COOR^{I}, —(CH₂)_{b}—COOH, —(CH₂)_{b}^{I}—COOR^{I}, —OR″^{I}, OH, —(CH₂)_{b}^{I}-phenyl, —O—(CH₂)₂—O—CH₃, —O—CH₂-(2,2-dimethyl-1,3-dioxolan-4-yl), —CO—NH phenyl,

—(CH₂)_{b}^{I}-5- or 6-membered aromatic heterocycle containing from 1 to 4 heteroatoms selected from nitrogen, oxygen and sulfur, each of said phenyl and said heterocycle being optionally substituted with one or more substituents selected from halogen, alkyl containing from 1 to 6 carbon atoms, alkoxy containing from 1 to 6 carbon atoms and CF₃,

R^{I}, R^{6I} and R^{7I} being as defined above,

R″^{I} being selected from alkyl radicals containing from 1 to 6 carbon atoms substituted with one or more radicals selected from hydroxy, protected hydroxy, oxo, halogen and cyano radicals,

a^{I} being equal to 0 or 1 and b^{I} being an integer from 0 to 6,

provided that, when R′^{I} is OH, R^{1I} is CONR^{6I}R^{7I} in which one of R^{6I} and R^{7I} is an alkoxy containing from 1 to 6 carbon atoms; or

b) R^{4I} is hydrogen or (CH₂)ₙ′^{I}₁R^{5I}, wherein n′^{I}₁, is 0, 1 or 2 and R^{5I} is as defined above,

and R^{1I} and R^{3I}, together with the carbons to which they are attached, form a substituted phenyl or heterocycle, as defined above;

and, in both cases a) and b), R^{2I} is selected from the group consisting of hydrogen, halogen, R^{I}, S(O)ₘ^{I}R^{I}, OR^{I}, NHCOR^{I}, NHCOOR^{I} and NHSO₂R^{I}, R^{I} being as defined above and m^{I} being 0, 1 or 2,

X^{I} is a divalent group —C(O)—B¹— linked to the nitrogen atom by the carbon atom,

B^{I} is a divalent group selected from 1) —NR^{8I}—(CH₂)ₙ″^{I}-linked to the carbonyl by the nitrogen atom, n″^{I} is 0 and R^{8I} is a radical selected from the group consisting of hydrogen, OH, R^{I}, OR^{I}, Y^{I}, OY¹, Y^{1I}, OY^{1I}, Y^{2I}, OY^{2I}, Y^{3I}, O—CH₂—CH₂—S(O—)ₘ^{I}—R^{I}, SiR^{aI}R^{bI}R^{cI} and OSiR^{aI}R^{bI}R^{cI}, wherein each of R^{aI}, R^{bI} and R^{cI} is a linear or branched alkyl containing from 1 to 6 carbon atoms or an aryl containing from 6 to 10 carbon atoms, and R^{I} and m^{I} are as defined above;

Y^{I} is selected from the group consisting of COH, COR^{I}, COOR^{I}, CONH₂, CONHR^{I}, CONHOH, CONHSO₂R^{I}, CH₂COOH, CH₂COOR^{I}, CHF—COOH, CHF—COOR^{I}, CF₂—COOH, CF₂—COOR^{I}, CN, CH₂CN, CH₂CONHOH, CH₂CONHCN, CH₂tetrazole, protected CH₂tetrazole, CH₂SO₃H, CH₂SO₂R^{I}, CH₂PO(OR^{I})₂, CH₂PO(OR^{I})(OH), CH₂PO(R¹)(OH) and CH₂PO(OH)₂;

Y^{1I} is selected from the group consisting of SO₂R^{I}, SO₂NHCOH, SO₂NHCOR^{I}, SO₂NHCOOR^{I}, SO₂NHCONHR^{I}, SO₂NHCONH₂ and SO₃H;

Y^{2I} is selected from the group consisting of PO(OH)₂, PO(OR^{I})₂, PO(OH)(OR^{I}) and PO(OH)(R^{I});

Y^{3I} is selected from the group consisting of tetrazole, tetrazole substituted with R^{I}, squarate, NH or NR^{I}tetrazole, NH or NR^{I}tetrazole substituted with R^{I}, NHSO₂R^{I}, NR^{I}SO₂R^{I}, CH₂tetrazole and CH₂tetrazole substituted with R^{I}, R^{I} being as defined above,

and n^{I} is 1, or one of its salts with a base or an acid.

Some β-lactamase inhibitors useful with the methods, compositions and compounds provided herein are described in U.S. Pat. No. 7,612,087, incorporated herein by reference in its entirety. Some embodiments include compounds having the following formula:

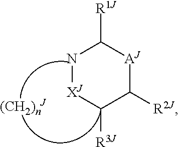

wherein, R^{1J} is hydrogen, COOH, CN, COOR^{J}, CONR^{6J}R^{7J}, (CH₂)ₙ′^{J}R^{5J} or C(═NR^{6J})NHR^{7J};

R^{J} is selected from the group consisting of alkyl containing 1 to 6 carbon atoms optionally substituted by a pyridyl or carbamoyl radical, —CH₂-alkenyl containing 3 to 9 carbon atoms, aryl containing 6 to 10 carbon atoms and aralkyl containing 7 to 11 carbon atoms, wherein the nucleus of said aryl or aralkyl is optionally substituted by OH, NH₂, NO₂, alkyl containing 1 to 6 carbon atoms, alkoxy containing 1 to 6 carbon atoms or by one or more halogen atoms;

R^{6J} and R^{7J} are identical or different and are independently selected from the group consisting of hydrogen, alkyl containing 1 to 6 carbon atoms, aryl containing 6 to 10 carbon atoms and aralkyl containing 7 to 11 carbon atoms optionally substituted by a carbamoyl, ureido or dimethylamino radical, and alkyl containing 1 to 6 carbon atoms substituted by a pyridyl radical;

n′^{J} is 1 or 2;

R^{5J} is selected from the group consisting of COOH, CN, OH, NH₂, CO—NR^{6J}R^{7J}, COOR^{J}, OR^{J}, OCHO, OCOR^{J}, OCOOR^{J}, OCONHR^{J}, OCONH₂, NHR^{J}, NHCOH, NHCOR^{J}, NHSO₂R^{J}, NH—COOR^{J}, NH—CO—NHR^{J} and NHCONH₂ wherein R^{J}, R^{6J} and R^{7J} are as defined above;

R^{2J} is hydrogen or (CH₂)ₙ′^{J}₁R^{5J} wherein n′^{J}₁ is 0, 1 or 2, and R^{5J} is as defined above;

R^{3J} is hydrogen or alkyl containing 1 to 6 carbon atoms;

A^{J} is a

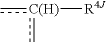

group wherein R^{4J} is hydrogen or (CH₂)ₙ′^{J}₁R^{5J} and n′^{J}₁ and R^{5J} are as defined above, and the dotted line is an optional bond with one of the two carbons which carry R^{1J} and R^{2J};

n^{J} is 1;

X^{J} is a divalent —C(O)—B^{J}— group linked to the nitrogen atom by the carbon atom wherein B^{J} is a divalent —O—(CH₂)ₙ″^{J}— group linked to the carbonyl by the oxygen atom, a divalent —NR^{8J}—(CH₂)ₙ″^{J}— or —NR^{8J}—O— group linked to the carbonyl by the nitrogen atom, n″^{J} is 0, and wherein B^{J} is —NR^{8J}—(CH₂)ₙ″^{J}—, R^{8J} is selected from the group consisting of hydrogen, OH, R^{J}, OR^{J}, Y^{J}, OY^{J}, Y^{1J}, OY^{1J}, Y^{2J}, OY^{2J}, Y^{3J}, OCH₂CH₂SOₘ^{J}R^{J}, OSiR^{aJ}R^{bJ}R^{cJ} and SiR^{aJ}R^{bJ}R^{cJ} and wherein B^{J} is — NR^{8J}—O—, R^{8J} is selected from the group consisting of hydrogen, R, Y^{J}Y^{2J}, Y^{3J} and SiR^{aJ}R^{bJ}R^{cJ}, wherein R^{aJ}, R^{bJ} and R^{cJ} is each independently a linear or branched alkyl containing 1 to 6 carbon atoms or aryl containing 6 to 10 carbon atoms, R^{J} is as defined above and m^{J} is 0, 1 or 2;

Y^{J} is selected from the group consisting of COH, COR^{J}, COOR^{J}, CONH₂, CONHR^{J}, CONHOH, CONHSO₂R^{J}, CH₂COOH, CH₂COOR^{J}, CH₂CONHOH, CH₂CONHCN, CH₂tetrazole, protected CH₂tetrazole, CH₂SO₃H, CH₂SO₂R^{J}, CH₂PO(OR^{J})₂, CH₂PO(OR^{J})(OH), CH₂PO(R^{J})(OH) and CH₂PO(OH)₂;

Y₁^{J} is selected from the group consisting of SO₂R^{J}, SO₂NHCOH, SO₂NHCOR^{J}, SO₂NHCOOR^{J}, SO₂NHCONHR^{J}, SO₂NHCONH₂ and SO₃H;

Y₂^{J} is selected from the group consisting of PO(OH)₂, PO(OR^{J})₂, PO(OH)(OR^{J}) and PO(OH)(R^{J});

Y₃^{J} is selected from the group consisting of tetrazole, tetrazole substituted by R^{J}, squarate, NH or NR^{J}-tetrazole, NH or NR^{J}-tetrazole substituted by R^{J}, NHSO₂R^{J} and NRSO₂R^{J} wherein R^{J} is as defined above; and

R^{1J}, R^{2J} and R^{3J} are not simultaneously hydrogen when n^{J} is 1,

R^{4J} is hydrogen and

X^{J} is —C(O)—O—(CH₂)ₙ″^{J} wherein n″^{J} is 0, or

X^{J} is —CO—NR^{8J}—(CH₂)ₙ″^{J} wherein n″^{J} is 0 and R^{8J} is hydrogen or phenyl.

Some β-lactamase inhibitors useful with the methods, compositions and compounds provided herein are described in Int. Pub. WO2011017125, incorporated herein by reference in its entirety. Some embodiments include compounds having the following formula:

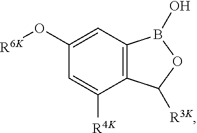

wherein, R^{3K} is —(CH₂)ₘ^{K} C(O)OR^{3aK},

m^{K} is an integer selected from 1, 2, 3, 4, 5, or 6;

R^{3aK} is selected from the group consisting of H, unsubstituted alkyl, and phenyl substituted alkyl;

R^{4K} is selected from the group consisting of unsubstituted alkyl, —OR^{4bK},

—(CH₂)ₙ^{K}—O—(CH₂)_{P}^{K}CH₃, and halogen

n^{K} is an integer selected from 1, 2, 3, 4, 5, or 6;

p^{K} is an integer selected from 0, 1, 2, 3, 4, 5, or 6;

R^{4bK} is H or substituted or unsubstituted alkyl;

R^{6K} is selected from the group consisting of H, substituted or unsubstituted alkyl, —C(O)OR^{6aK}, —C(O)NR^{6aK}R^{5bK}, —S(O₂)R^{6cK}, and A^{K};

R^{6aK} is H or unsubstituted alkyl;

R^{6bK} is H or unsubstituted alkyl;

R^{6cK} is selected from the group consisting of unsubstituted alkyl, NH₂ and heteroaryl, optionally substituted with unsubstituted alkyl A is selected from the group consisting of substituted or unsubstituted cycloalkyl, substituted or unsubstituted heterocycloalkyl, substituted or unsubstituted aryl and substituted or unsubstituted heteroaryl;

or a salt, hydrate or solvate thereof

Some β-lactamase inhibitors useful with the methods, compositions and compounds provided herein are described in Int. Pub. WO2009140309, incorporated herein by reference in its entirety. Some embodiments include compounds having the following formula:

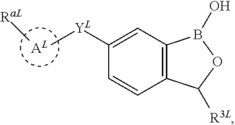

wherein, A^{L} is a member selected from cycloalkyl, heterocycloalkyl, aryl and heteroaryl;

Y^{L} is a member selected from 0 and —S(O)₂NH—

wherein the sulfur in —S(O)₂NH— is covalently attached to A^{L};

R^{3L} is a member selected from H, cyano and substituted alkyl;

R^{aL} is a member selected from H, —OR^{10L}, —NR^{10L}R^{11L}, —SR^{10L}, —S(O)R^{10L}, —S(O)₂R^{10L}, —S(O)₂NR^{10L}R^{11L}, —C(O)R^{10L}, —C(O)OR^{10L}, —C(O)NR^{10L}R^{11L}, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl,

each R^{10L} and each R^{11L} is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl

with the proviso that R^{10L} and R^{11L} together with the nitrogen to which they are attached, are optionally combined to form a 5- to 7-membered substituted or unsubstituted heterocycloalkyl ring; with the proviso that when Y^{L} is O, R^{L} is a member selected from cyano and substituted alkyl;

with the proviso that when Y^{L} is —S(O)₂NH—, R^{3L} is H, and R^{aL} is not H or unsubstituted alkyl or halosubstituted alkyl and salts thereof.

Some β-lactamase inhibitors useful with the methods, compositions and compounds provided herein are described in Int. Pub. WO2010130708, incorporated herein by reference in its entirety. Some embodiments include compounds having the following formula:

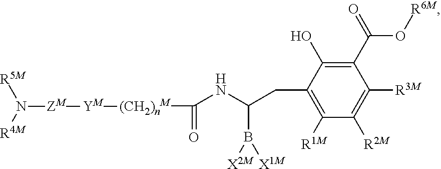

wherein, R^{1M}, R^{2M}, and R^{3M} are independently hydrogen, or selected from the group consisting of hydroxyl, halogen, carboxyl, cyano, thiol, optionally substituted: C₁-C₅ alkyl, C₁-C₅ alkoxy, C₁-C₅ alkenyl, C₃-C₆ cycloalkyl, C₃-C₆ heterocyclyl, amino, sulfide, and sulfone;

n^{m} is O, 1, or 2;

Y^{M} is selected from the group consisting of: (a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, (b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and (c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;

R^{4M} is hydrogen, or selected from the group consisting of: (a) C₁-C₅ alkyl any carbon of which can be substituted with from 0 to 3 substituents selected from the group consisting of hydroxyl, halogen, carboxyl, cyano, oxo, optionally substituted: alkyl, alkenyl, alky ny I₁ cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C₁-C₅ carbons comprise part of said oxyimino group, imino wherein any of the C₁-C₅ carbons comprise part of said imino group, amidino wherein any of the C₁-C₅ carbons comprise part of said amidino group, sulfido, and sulfoxido, (b) C₃-C₆ cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfide and sulfoxido, (c) heteroaryl group substituted with from 0 to 3 substituents selected from the group consisting of hydroxyl, halogen, carboxyl, cyano, thiol, sulfonic acid, sulfate, optionally substituted: alkyl, alkenyf, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and (d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;

R^{5M} is a lone pair of electrons, hydrogen, or selected from the group consisting of: (a) C₁-C₅ alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C₁-C₅ carbons comprise part of said oxyimino group, imino wherein any of the C₁-C₅ carbons comprise part of said imino group, amidino wherein any of the C₁-C₅ carbons comprise part of said amidino group, sulfido, and sulfoxido, (b) C₃-C₆ cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido, (c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and (d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;

or R^{4M} and Y^{M} together form a ring of between 5 and 7 atoms where said ring is optionally fused or spiro in relation to the ring system of Y^{M}, said ring optionally being partially saturated or aromatic and optionally containing 1-2 additional heteroatoms selected from the group consisting of N, O, S, and a combination thereof;

or R^{4M} and R^{5M} together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;

R^{6M} is hydrogen or an ester prodrug of the carboxylic acid;

Z^{M} is a bond;

or Z^{M} is optionally substituted: C₁-C₄ alkyl, C₁-C₄ alkoxy, C₁-C₄ sulfido, C₃-C₆ cycloalkyl, C₃-C₆ heterocyclyl where the bond to Y is through a carbon atom of said heterocyclyl ring, heteroaryl where the bond to Y is through a carbon atom of said heteraryl ring, oxyimino, imino, or amidino where the carbon of said oxyimino, imino, or amidino group is attached to Y;

or Z^{M} and Y^{M} together form a ring of 5-7 atoms where said ring is optionally fused or spiro in relation to the ring system of Y^{M}, said ring optionally being partially saturated or aromatic and optionally containing 1-3 heteroatoms selected from the group consisting of N, O, S, and a combination thereof;

or Z^{M} and R^{4M} together form a ring of 4-7 atoms where said ring optionally is saturated, partially unsaturated, or aromatic and optionally contains 1-2 additional heteroatoms selected from the group consisting of N, O, S, and a combination thereof;

X^{1M} and X^{2M} are independently hydroxyl, halogen, NR^{4M}R^{5M}, C₁-C₆ alkoxy, or when taken together X^{1M} and X^{2M} form a cyclic boron ester where said chain or ring contains from 2 to 20 carbon atoms and, optionally, 1-3 heteroatoms selected from the group consisting of N, O, S, and a combination thereof, or when taken together X^{1M} and X^{2M} form a cyclic boron amide where said chain or ring contains from 2 to 20 carbon atoms and, optionally, 1-3 heteroatoms selected from the group consisting of N, O, S, and a combination thereof, or when taken together X^{1M} and X^{2M} form a cyclic boron amide-ester where said chain contains from 2-20 carbon atoms and, optionally, 1-3 heteroatoms selected from the group consisting of N, O, S, and a combination thereof, or X^{1M} is hydroxyl and X^{2M} is replaced by the ortho-hydroxyl oxygen of the phenyl ring such that a 6-membered ring is formed;

or a salt thereof;

provided that when R^{1M}, R^{2M}, R^{3M}, R^{4M}, R^{5M} and R^{6M} are hydrogen, X^{1M} and X^{2M} are hydroxyl, n^{M} is 0, Y^{M} is phenyl, and Z^{M} is CH₂ then Z^{M} cannot be at the meta-position of the phenyl ring relative to the rest of the molecule.

Some β-lactamase inhibitors useful with the methods, compositions and compounds provided herein are described in Int Pub. WO2009064413, incorporated herein by reference in its entirety. Some embodiments include compounds having the following formula:

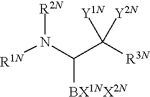

wherein, R^{1N} is —C(O)R^{4N}: —C(O)NR^{4N}R^{5N}; —C(O)OR^{4N}; —S(O)₂R^{4N}, —C(═NR^{4N}R^{5N})R^{4N}, —C(═N R^{4N}R^{5N}) N R^{4N}R^{5N}, hydrogen, or is selected from the group consisting of: (a) aryl group substituted with from O to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (b) heteroaryl group substituted with from O to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (c) heterocyclic group substituted with from O to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;

R^{2N} is hydrogen, or is selected from the group consisting of: (a) C₁-C₆ alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₆ carbons comprise part of said oxyimino group, sulfido, and sulfoxido, (b) C₃-C₇ cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

R^{3N} is an aryl or heteroaryl group substituted with from 1 to 4 substituents selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol, provided that, when one of the substituents is a carboxylic acid group located at the 3-position relative to the group containing Y^{1N} and Y^{2N}, one of the remaining substituents is not a hydroxyl or amino group located at the 2- or 6-position relative to the group containing Y^{1N} and Y^{2N};

R^{4N} is selected from the group consisting of: (a) C₁-C₁₀ alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₁₀ carbons comprise part of said oxyimino group, sulfido, and sulfoxido, (b) C₃-C₁₀ cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

R^{5N} is hydrogen or is selected from the group consisting of: (a) C₁-C₆ alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₁₀ carbons comprise part of said oxyimino group, sulfido, and sulfoxido, (b) C₃-C₇ cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

X^{1N} and X^{2N} are independently hydroxyl, halogen, NR^{4N}R^{5N}, C₁-C₆ alkoxy, or when taken together X^{1N} and X^{2N} form a cyclic boron ester where said chain or ring contains from 2 to 20 carbon atoms and, optionally, 1-3 heteroatoms which can be O, N, or S, or when taken together X^{1N} and X^{2N} form a cyclic boron amide where said chain or ring contains from 2 to 20 carbon atoms and, optionally, 1-3 heteroatoms which can be O, N, or S, or when taken together X^{1N} and X^{2N} form a cyclic boron amide-ester where said chain contains from 2-20 carbon atoms and, optionally, 1-3 heteroatoms which can be O, N, or S, or X^{1N} and R^{1N}N together form a cyclic ring where said ring contains 2 to 10 carbon atoms and, optionally, 1-3 heteroatoms which can be O, N, or S, and X^{2N} is hydroxyl, halogen, NR^{4N}R^{5N}, C₁-C₆ alkoxy, or X^{1N} and R^{3N} together form a cyclic ring where said ring contains 2 to 10 carbon atoms and, optionally, 1-3 heteroatoms which can be O, N, or S, and X^{2N} is hydroxyl, halogen, NR^{4N}R^{5N}, or C₁-C₆ alkoxy;

Y^{1N} and Y^{2N} are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y^{1N} and Y^{2N} form a cyclic structure containing from 3-12 carbon atoms and, optionally, 1-3 heteroatoms which can be O, N, or S;

or a salt thereof;

provided that, when R^{1N}N is —C(O)R^{4N}, R^{2N}N is hydrogen, R^{3N} is a phenyl group having one substitution consisting of a carboxylic acid group located at the 3-position relative to the group containing Y^{1N} and Y^{2N}, X^{1N} and X^{2N} are hydroxyl, and Y^{1N} and Y^{2N} are hydrogen, R^{4N} is not unsubstituted C₁ alkyl or C₁ alkyl having one substitution consisting of a phenyl group.

Some β-lactamase inhibitors useful with the methods, compositions and compounds provided herein are described in Int. Pub. WO2009064414, incorporated herein by reference in its entirety. Some embodiments include compounds having the following formula:

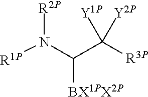

wherein, R^{1P} is —C(O)R^{4P}; —C(O)NR^{4P}R^{5P}; —C(O)OR^{4P}; —S(O)₂R^{4P}, —C(═NR^{4P}R^{5P})R^{4P}, —C(═NR^{4P}R^{5P})NR^{4P}R^{5P}, hydrogen, or is selected from the group consisting of: (a) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (b) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (c) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido;

R^{2P} hydrogen, or is selected from the group consisting of: (a) C₁-C₆ alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₆ carbons comprise part of said oxyimino group, sulfido, and sulfoxido, (b) C₃-C₇ cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxide), and (e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

R^{3P} is an aryl or heteroaryl group substituted with from 1 to 4 substituents wherein one of the substituents is a hydroxyl or amino group located at the 2 position relative to the group containing Y^{1P} and Y^{2P}, and wherein the remaining substituents are selected from the group consisting of hydroxyl, alkyl, cycloalkyl, alkoxy, alkenyl, alkynyl, amino, aminocarbonyl, carbonyl, aminosulfonyl, alkylaryl, aryl, aryloxy, carboxyl, cyano, guanidino, halogen, heteroaryl, heterocyclyl, sulfido, sulfonyl, sulfoxido, sulfonic acid, sulfate, and thiol;

R^{4P} is selected from the group consisting of: (a) C₁-C₁₀ alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₁₀ carbons comprise part of said oxyimino group, sulfido, and sulfoxido, (b) C₃-C₁₀ cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

R^{5P} is hydrogen or is selected from the group consisting of: (a) C₁-C₆ alkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the C₁-C₁₀ carbons comprise part of said oxyimino group, sulfido, and sulfoxido, (b) C₃-C₇ cycloalkyl any carbon of which can be substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, sulfido, and sulfoxido, (c) aryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, (d) heteroaryl group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, sulfido, and sulfoxido, and (e) heterocyclic group substituted with from 0 to 3 substituents selected from the group consisting of hydroxyl, halogen, carboxyl, cyano, thiol, sulfonic acid, sulfate, optionally substituted: alkyl, cycloalkyl, alkoxy, alkenyl, alkynyl, aryl, heteroaryl, heterocyclyl, arylalkyl, alkylaryl, heteroarylalkyl, alkylheteroaryl, cycloalkoxy, heterocyclyloxy, aryloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, sulfido, and sulfoxido;

X^{1P} and X^{2P} are independently hydroxyl, halogen, NR^{4P}R^{5P}, C₁-C₆ alkoxy, or when taken together X^{IP} and X^{2P} form a cyclic boron ester where said chain or ring contains from 2 to 20 carbon atoms and, optionally, 1-3 heteroatoms which can be O, N, or S, or when taken together X^{1P} and X^{2P} form a cyclic boron amide where said chain or ring contains from 2 to 20 carbon atoms and, optionally, 1-3 heteroatoms which can be O, N, or S, or when taken together X^{1P} and X^{2P} form a cyclic boron amide-ester where said chain contains from 2-20 carbon atoms and, optionally, 1-3 heteroatoms which can be O, N, or S, or X^{1P} and R^{1P} together form a cyclic ring where said ring contains 2 to 10 carbon atoms and, optionally, 1-3 heteroatoms which can be O, N, or S, and X^{2P} is hydroxyl, halogen, NR^{4P}R^{5P}, C₁-C₆ alkoxy, or X^{1P} and R^{3P} together form a cyclic ring where said ring contains 3 to 10 carbon atoms and, optionally, 1-3 heteroatoms which can be O, N, or S, and X^{2P} is hydroxyl, halogen, NR^{4P}R^{5P}, or C₁-C₆ alkoxy;

Y^{1P} and Y^{2P} are independently hydrogen, alkyl, cycloalkyl, alkoxy, alkenyl, alkynyl, amino, aminosulfonyl, aminocarbonyl, carbonyl, alkylaryl, aryl, aryloxy, carboxyl, cyano, halogen, heteroaryl, heteroaryloxy, heterocyclyl, sulfido, sulfonyl, or sulfoxido, or taken together Y^{1P} and Y^{2P} form a cyclic structure containing from 3-12 carbon atoms and, optionally, 1-3 heteroatoms which can be O, N, or S;

or a salt thereof;

provided that, when le is —C(O)R^{4P}, R^{2P} is hydrogen, R^{3P} is a phenyl group having two substituents consisting of a hydroxyl at the 2-position and a carboxylic acid at the 3-position relative to the group containing Y^{1P} and Y^{2P}, X^{1P} and X^{2P} are hydroxyl or X^{1P} is hydroxyl and X^{2P} is replaced by the ortho-hydroxyl oxygen of R^{3P} such that a 6-membered ring is formed, and Y^{1P} and Y^{2P} are hydrogen, R^{4P} is not unsubstituted C₁ alkyl.

Some β-lactamase inhibitors useful with the methods, compositions and compounds provided herein are described in Int. Pub. WO2009091856, incorporated herein by reference in its entirety. Some embodiments include compounds having the following formula:

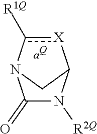

or a pharmaceutically acceptable salt thereof, wherein the bond identified as a^{Q} is a single bond or a double bond;

when bond a^{Q} is a single bond, X is CH₂, CH₂CH₂, CH₂CH₂CH₂, CH—CH₅, CH₂—CH—CH, or CH═CH—CH₂;

when bond a^{Q} is a double bond, X is CH, CH—CH₂, or CH—CH═CH;

R^{1Q} is C(O)N(R^{3Q})R^{4Q}, C(O)OR^{3Q}, or C(O)OR^{5Q};

R^{2Q} is SO₃M^{Q}, OSO₃M^{Q}, SO₂NH₂, PO₃M^{Q}, OPO₃M^{Q}, CH₂CO₂M^{Q}, CF₂CO₂M^{Q}, or CF₃;

M^{Q} is H or a pharmaceutically acceptable cation;

R^{3Q} is (1) C 1-8 alkyl substituted with a total of from 1 to 4 substituents selected from the group consisting of zero to 2 N(R^{AQ})R^{BQ}, zero to 2 R^{CQ}, and zero to 1 of AryA^{Q}, HetA^{Q}, or HetB^{Q}, (2) CycA^{Q}, (3) HetA^{Q}, (4) AryA^{Q}, (5) HetB^{Q}, or (6) AryB^{Q};

R^{4Q} is H or C 1-8 alkyl optionally substituted with N(R^{AQ})R^{BQ};

or alternatively, when R^{1Q} is C(O)N(R^{3Q})R^{4Q}, R^{3Q} and R^{4Q} together with the N atom to which they are both attached form a 4- to 9-membered, saturated monocyclic ring optionally containing 1 heteroatom in addition to the nitrogen attached to R^{3Q} and R^{4Q} selected from N, O, and S, where the S is optionally oxidized to S(O) or S(O)₂; wherein the monocyclic ring is optionally fused to, bridged with, or spiro to a 4- to 7-membered, saturated heterocyclic ring containing from 1 to 3 heteroatoms independently selected from N, O and S, where the S is optionally oxidized to S(O) or S(O)₂, to form a bicyclic ring system, wherein the monocyclic ring or the bicyclic ring system so formed is optionally substituted with 1 or 2 substituents each of which is independently: (1) C₁₋₆ alkyl, (2) C₁₋₆ fluoroalkyl, (3) (CH₂)₁₋₂G, wherein G is OH, O—C₁₋₆ alkyl, O—C₁₋₆ fluoroalkyl, N(R^{AQ})R^{BQ}, C(O)N(R^{AQ})R^{BQ}, C(O)R^{AQ}, CO₂R^{AQ}, or SO₂R^{AQ}, (4) O—C₁₋₆ alkyl, (5) O—C₁₋₆ fluoroalkyl, (6) OH, (7) oxo, (8) halogen, (9) N(R^{AQ})R^{BQ}, (10) C(O)N(R^{AQ})R^{BQ}, (11) C(O)R^{AQ}, (12) C(O)—C₁₋₆ fluoroalkyl, (13) C(O)OR^{AQ}, or (14) S(O)₂R^{AQ};

R^{5Q} is C₁₋₈ alkyl substituted with 1 or 2 substituents each of which is independently N(R^{AQ})C(O)-AryA^{Q};

CycA^{Q} is C₄₋₉ cycloalkyl which is optionally substituted with a total of from 1 to 4 substituents selected from zero to 2 (CH₂)ₙ^{Q}N(R^{AQ})R^{BQ} and zero to 2 (CH₂)ₙ^{Q}R^{CQ};

HetA^{Q} is a 4- to 9-membered saturated or mono-unsaturated heterocyclic ring containing from 1 to 3 heteroatoms independently selected from N, O and S, wherein any ring S is optionally oxidized to S(O) or S(O)₂ and either 1 or 2 ring carbons are optionally oxidized to C(O); wherein the ring is optionally fused with a C₃₋₇ cycloalkyl; and wherein the optionally fused, saturated or mono-unsaturated heterocyclic ring is optionally substituted with a total of from 1 to 4 substituents selected from zero to 2 (CH₂)ₙ^{Q}N(R^{AQ})R^{BQ} and zero to 2 (CH₂)ₙ^{Q}R^{CQ};

AryA^{Q} is phenyl which is optionally substituted with a total of from 1 to 4 substituents selected from zero to 2 (CH₂)ₙ^{Q}N(R^{AQ})R^{BQ} and zero to 2 (CH₂)ₙ^{Q}R^{CQ};

HetB^{Q} is a 5- or 6-membered heteroaromatic ring containing from 1 to 4 heteroatoms selected from 1 to ₃ N atoms, zero or 1 O atom, and zero or 1 S atom; wherein the heteroaromatic ring is optionally fused with a 5- to 7-membered, saturated heterocyclic ring containing 1 or 2 heteroatoms independently selected from N, O and S, wherein any ring S is optionally oxidized to S(O) or S(O)₂ and either 1 or 2 non-fused ring carbons are optionally oxidized to C(O); and wherein the optionally fused heteroaromatic ring is optionally substituted with a total of from 1 to 4 substituents selected from zero to 2 (CH₂)ₙ^{Q}N(R^{AQ})R^{BQ} and zero to 2 (CH₂)ₙ^{Q}R^{CQ};

AryB^{Q} is a bicyclic ring system which is phenyl fused with a 5- to 7-membered saturated heterocyclic ring containing from 1 to 3 heteroatoms independently selected from N, O and S, wherein any ring S is optionally oxidized to S(O) or S(O)₂, and wherein the bicyclic ring system is optionally substituted with a total of from 1 to 4 substituents selected from zero to 2 2 (CH₂)ₙ^{Q}N(R^{AQ})R^{BQ} and zero to 2 (CH₂)ₙ^{Q}R^{CQ};

each n^{Q} is independently an integer which is 0, 1, 2, or 3;

each R^{AQ} is independently H or C₁₋₈ alkyl;

each R^{BQ} is independently H or C₁₋₈ alkyl;

each R^{CQ} is independently C₁₋₆ alkyl, OH, O—C₁₋₈ alkyl, OC(O)—C₁₋₈ alkyl, C(═NH)NH₂, NH—C(═NH)NH₂, halogen, CN, C(O)R^{AQ}, C(O)OR^{AQ}, C(O)N(R^{AQ})R^{BQ}, SO₂R^{AQ}, SO₂N(R^{AQ})R^{BQ}, pyridyl, pyrrolidinyl, piperidinyl, piperazinyl, morpholinyl, or thiomorpholinyl; and

provided that:

(A) when R^{1Q} is C(O)OR^{3Q} and R^{3Q} is AryA^{Q}, then AryA^{Q} is not (i) unsubstituted phenyl, (ii) phenyl substituted with NH₂, (iii) phenyl substituted with OH, (iii) phenyl substituted with O—C₁₋₆ alkyl, (iv) phenyl substituted with one or more halogens, or (v) phenyl substituted with C₁₋₆ alkyl;

(B) when R^{1Q} is C(O)OR^{3Q} and R^{3Q} is C₁₋₆ alkyl substituted with HetB^{Q}, then HetB^{Q} is not pyridyl;

(C) when R^{1Q} is C(O)OR^{3Q} and R^{3Q} is CH₂-AryA^{Q} or CH₂CH₂-AryA^{Q}, then AryA^{Q} is not (i) unsubstituted phenyl, (ii) phenyl substituted with NH₂, OH, O—C₁₋₆ alkyl, or C₁₋₆ alkyl, or (iii) phenyl substituted with one or more halogens;

(D) when R^{1Q} is C(O)N(R^{3Q})R^{4Q}, R^{3Q} is AryA^{Q}, CH₂-AryA^{Q} or CH₂CH₂-AryA^{Q}, and R^{4Q} is H or C₁₋₆ alkyl, then AryA^{Q} is not unsubstituted phenyl, phenyl substituted with N(CH₃)₂, or phenyl substituted with C(O)NH₂;

(E) when R^{1Q} is C(O)N(R^{3Q})R^{4Q}, R^{3Q} is C₁₋₆ alkyl substituted with HetB^{Q}, and R^{4Q} is H or C₁₋₆ alkyl then HetB^{Q} is not pyridyl; and

(F) when R^{1Q} is C(O)OR^{3Q} and R^{3Q} is C₁₋₆ alkyl substituted with R^{CQ}, then R^{CQ} is not C(O)NH₂.

Some β-lactamase inhibitors useful with the methods, compositions and compounds provided herein include the compound MK-7655, having the following formula:

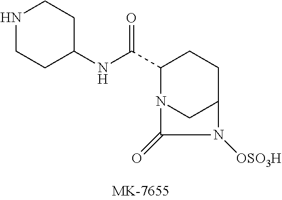

Some β-lactamase inhibitors useful with the methods, compositions and compounds provided herein are described in Int. Pub. WO2008039420, incorporated herein by reference in its entirety. Some embodiments include compounds having the following formula:

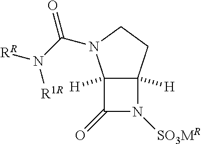

or a pro-drug or pharmaceutically acceptable salt thereof,

wherein, R^{R} represents a 7-, 8-, or 9-membered saturated or unsaturated ring optionally containing from 1 to 3 heteroatoms independently selected from N, O and S, wherein the ring is optionally substituted with one or more R^{aR} groups;

R^{1R} represents hydrogen or methyl;

each R^{aR} independently represents hydrogen, C₁₋₆ alkyl, halo, —(CH₂)ₙ^{R}CN, —(CH2)ₙ^{R}NO₂, —(CH₂)ₙ^{R}OR^{bR}, —(CH₂)ₙ^{R}SR^{bR}, —(CH₂)ₙ^{R}N(R^{bR})₂, —(CH₂)ₙ^{R}C(O)N(R^{bR})₂, —(CH₂)ₙ^{R}SO₂N(R^{bR})₂, —(CH₂)ₙ^{R}CO₂R^{bR}, —(CH₂)n^{R}C(O)R^{bR}, —(CH₂)ₙ^{R}OC(O)R^{bR}, —(CH₂)ₙ^{R}NHC(O)R^{bR}, —(CH₂)ₙ^{R}NHC(O)₂R^{bR}, —(CH₂)ₙ^{R}NHSO₂R^{bR}, —(CH₂)ₙ^{R}C(═NH)NH₂, or —(CH₂)ₙ^{R}C(═NH)H; or two R^{aR} groups on the same ring carbon atom are optionally taken together to form oxo; or two R^{aR} groups on the same ring sulfur atom are optionally taken together with the sulfur to represent SO; or four R^{aR} groups on the same ring sulfur atom are optionally taken together with the sulfur to represent SO₂;

each n^{R} is independently 0, 1, 2, 3, or 4;

each R^{bR} independently represents hydrogen or C₁₋₄ alkyl; and

M^{R} represents hydrogen or a pharmaceutically acceptable cation or, when the compound contains an internal base which is capable of being protonated by a sulfonic acid, M^{R} is optionally a negative charge.

Some β-lactamase inhibitors useful with the methods, compositions and compounds provided herein include compound BAL-29880, having the following formula:

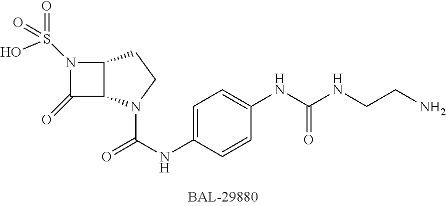 [SMILES: NCCNC(=O)Nc1ccc(NC(=O)N2CC[C@@H]3[C@H]2C(=O)N3S(=O)(=O)O)cc1]

Some β-lactamase inhibitors useful with the methods, compositions and compounds provided herein are described in Int. Pub. WO0222137, incorporated herein by reference in its entirety. Some embodiments include compounds having the following formula:

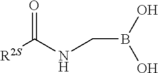

wherein, R^{2S} is H, alkyl, heteroalkyl, cycloalkyl, heterocycloalkyl, alkyl-cycloalkyl, heteroalkyl-cycloalkyl, alkyl-heterocycloalkyl, heteroalkyl-heterocycloalkyl, alkenyl, heteroalkenyl, cyclic alkene, heterocyclic alkene, alkyl-cyclic alkene, heteroalkyl-cyclic alkene, cyclic alkene-alkyl, cyclic alkene-heteroalkyl, alkyl-heterocyclic alkene, heterocyclic alkene-alkyl, heterocyclic alkene-heteroalkyl, heteroalkyl-heterocyclic alkene, alkyl-O-cyclic alkene, alkyl-O-heterocyclic alkene, alkyl-S-cyclic alkene, alkyl-S-heterocyclic alkene, or

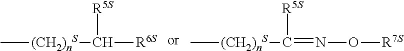

each R^{2S} may be unsubstituted or substituted with one or more R^{3S} groups;

each R^{3S} is independently alkyl, heteroalkyl, cyclic alkene, cyclic alkene substituted with one or more R^{4S} groups, heterocyclic alkene, heterocyclic alkene substituted with one or more R^{4S} groups, halogen, —NH₂, ═NH, ═N, ═N—OH, ═O, —OH, —O—C(O)H, —O-alkyl, —COOH, —(CH₂)ₘ^{S}—COOH, ═CH—(CH₂)ₘ^{S}—COOH, —CN, ═N—O—CH₃, ═N—O—C(CH₃)₂—COOH, ═N—O—C(CH₃)₂—C(O)—O-alkyl, —(CH₂)ₘ^{S}—NH₂, ═C(COOH)—C(O)—NH₂, —C(O)—O-alkyl, —C(O)—O-cyclic alkene, —S-alkyl, —SO₃H, or —SO₂—CH₃;

each R^{4S} is independently alkyl, halogen, ═NH, —NH₂, —(CH₂)ₘ^{S}—NH₂, ═O, —OH, —(CH₂)ₘ^{S}— OH, —COOH, —(CH₂)ₘ^{S}—COOH, —C(═O)NH₂, —SO₃H, or —SO₂—CH₃;

R^{5S} is cyclic alkene or heterocyclic alkene, each of which may be unsubstituted or substituted with one or more R^{4S} groups;

R^{6S} is alkyl or heteroalkyl, each of which may be unsubstituted or substituted with one or more R^{4S} groups;

R^{7S} is H or R^{7S} is alkyl or heteroalkyl, each of which may be unsubstituted or substituted with one or more R^{4S} groups;

m^{S} is 1-4; and

n^{S} is 0-2;

or a pharmaceutically-acceptable salt thereof

Some β-lactamase inhibitors useful with the methods, compositions and compounds provided herein are described in Int. Pub. WO2000035904, incorporated herein by reference in its entirety. Some embodiments include compounds having the following formula:

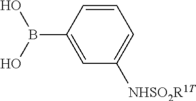

wherein, R^{1T} is N-lower alkyl, a cyclic alkene or a heterocyclic alkene, wherein the cyclic alkene and heterocyclic alkene may be substituted with one or more substituents R^{2T}; and

each R^{2T} is independently H, a halogen atom, lower, alkyl, lower alkyl substituted with one or more halogen atoms, NH₂, NO, NO₂, N-lower alkyl, N-lower alkyl substituted with one or more halogen atoms, OH, O-lower alkyl, O-lower alkyl substituted with one more halogen atoms, CO-lower alkyl, CO-lower alkyl substituted with one or more halogen atoms, COOH, lower alkyl-COOH, COO-lower alkyl, CONH₂, CON-lower alkyl, SO₃H, SO₂NH₂, SO₂N-lower alkyl, or B(OH)₂, except that R^{2T} cannot be N-lower alkyl when R^{1T} is naphthalene;

or a pharmaceutically-acceptable salt thereof

Some β-lactamase inhibitors useful with the methods, compositions and compounds provided herein are described in Int. Pub. WO98/56392, incorporated herein by reference in its entirety. Some embodiments include compounds having the following formula:

(OH)₂B—R^{U}

wherein, R^{U} is naphthalene, phenanthrene, or has one of the following formulas:

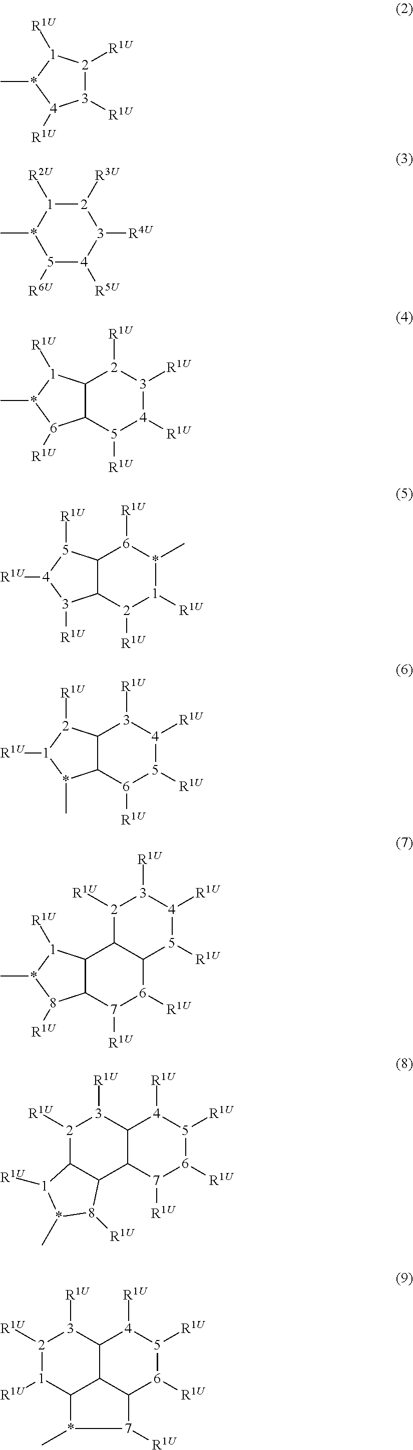

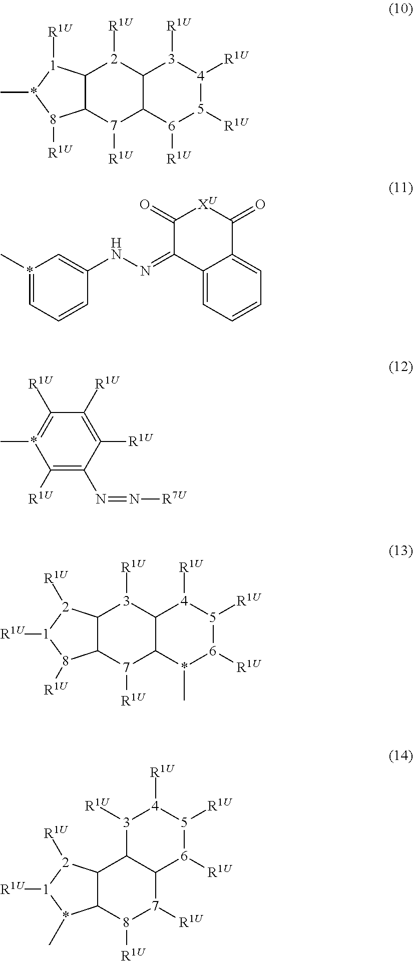

wherein, ring system (2), (3), (4), (5), (6), (7), (8), (9) or (10) is aromatic or nonaromatic; the atom center * is (R) or (S) in the case of chiral compounds; positions 1, 2, 3, 4, 5, 6, 7 or 8 each independently is C, N, O or S;

R^{1U} through R^{6U} each independently is a lone pair, H, B(OH)₂, a halogen atom, CF₃, CH₂CF₃, CCl₃, CH₂CCl₃, CBR^{3U}, CH₂CBR^{3U}, NO₂, lower alkyl, CO₂H, CHCHCOOH, CH2CH₂CH₂COOH, SO₃H, PO₃H, OSO₃H, OPO₃H, OH, NH₂, CONH₂, COCH₃, OCH₃, or phenyl boronic acid, except that R^{2U}, R^{3U}, R^{4U}, R^{5U} and R^{6U} cannot all simultaneously be H, R^{2U} cannot be lower alkyl when R^{3U}, R^{4U}, R^{5U} and R^{6U} are H, R^{3U} cannot be NH₂, OH or lower alkyl when R^{2U}, R^{4U}, R^{5U}, R^{6U} are H, and R^{4U} cannot be lower alkyl when R^{2U}, R^{3U}, R^{5U} and R^{6U} are H;

R^{7U} is a lone pair of electrons, H, B(OH)₂, a halogen atom, CF₃, CCl₃, CBR^{3U}, CH₂CF₃, CH₂CCl₃, CH₂CBR^{3U}, NO₂, CONH₂, COCH₃, OCH₃, lower alkyl, aryl, aryl substituted with one or more substituents R^{8U}, heteroaryl, or heteroaryl substituted with one or more substituents R^{8U};

each R^{8U} is independently a lone pair, H, B(OH)₂, a halogen atom, CF₃, CCl₃, CBR^{3U}, CH₂CF₃, CH₂CCl₃, CH₂CBR^{3U}, NO₂, lower alkyl, O, N, S, OH, NH₂, N(CH₃)₂, N(CH₃)CH₂CH₃, NCOCH₃, COOH, CHCHCOOH, CH₂CH₂CH₂COOH, CONH₂, COCH₃, OCH₃, OCl or phenyl boronic acid;

X^{U} is O, NH, NCH₃ or

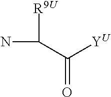

Y^{U} is OH, NH₂, NCH₃, N(CH₃)₂, NHCOCH₃ or NHCOCH₂COOH; and

R^{9U} is a lone pair of electrons, H, B (OH)₂, a halogen atom, CF₃, CCl₃, CBR^{3U}, CH₂CF₃, CH₂CCl₃, CH₂CBR^{3U}, NO₂, CO₂H, CHCHCOOH, CH₂CH₂CH₂COOH, SO₃H, PO₃H, OSO₃H, OPO₃H, OH, NH₂, CONH₂, COCH₃, OCH₃, phenyl boronic acid, lower alkyl, or a side chain of a standard amino acid;

or a pharmaceutically-acceptable salt thereof.

Some β-lactamase inhibitors useful with the methods, compositions and compounds provided herein are described in Int. Pub. WO200035905, incorporated herein by reference in its entirety. Some embodiments include compounds having the following formula:

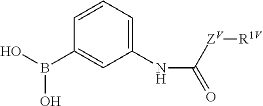

wherein, R^{1V} is lower alkyl, lower alkyl substituted with one or more halogen atoms, a cyclic alkene, or a heterocylic alkene, wherein the cyclic alkene or heterocyclic alkene may be substituted with one or more substituents R^{2V};

each R^{2V} is independently H, a halogen atom, lower alkyl, lower alkyl substituted with one or more halogen atoms, NH₂, NO, NO₂, CN, N-lower alkyl, N-lower alkyl substituted with one or more halogen atoms, OH, O-lower alkyl, O-lower alkyl substituted with one or more halogen atoms, CO-lower alkyl, CO-lower alkyl substituted with one or more halogen atoms, COOH, lower alkyl-COOH, CONH₂, CON-lower alkyl, SO₃H, SO₂NH₂, or SO₂N-lower alkyl; and

Z^{V} is a bond, O, S, lower alkyl radical, or lower heteroalkyl radical;

or a pharmaceutically-acceptable salt thereof.

Some β-lactamase inhibitors useful with the methods, compositions and compounds provided herein are described in Int. Pub. WO2007065288 and U.S. Pub. No. 2010/0056478, the disclosures of which are incorporated herein by reference in their entireties. Some embodiments include compounds having the following formula:

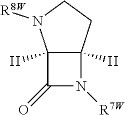

wherein, R^{7W} signifies SO₃H, OSO₃H or OCR^{jW}R^{jW′}COOH,

wherein R^{jW} and R^{j′W} are independently selected from hydrogen; alkyl; phenyl which may be substituted with 1 to 5 substituents selected from alkyl, hydroxyl, alkylhydroxyl, amino, alkylamino, dialkylamino and halogen; benzyl which may be substituted with 1 to 5 substituents selected from alkyl, hydroxyl, alkylhydroxyl, amino, alkylamino, dialkylamino and halogen; alkylamino and alkoxyalkyl;

R^{8W} is alkoxycarbonylamino, the acyl residue of an a or β-amino acid, or a residue of the formula Q^{W}-(X^{W})ᵣ^{W}—Y^{W}—, wherein Q^{W} is a 3-6 membered ring which optionally contains nitrogen, sulphur and/or oxygen and which is optionally fused to a phenyl ring or to a 5-6 membered heterocyclic ring and which is optionally substituted with 1 to 4 substituents selected from alkyl, allyl, hydroxyl, alkylhydroxyl, amino, alkylamino, dialkylamino, carboxamide, which may be substituted, carboxylic acid, carbonylalkoxy, aminocarbonyl, alkylaminocarbonyl halogen, halogenomethyl, dihalogenomethyl, trihalogenomethyl, sulfamide, substituted sulfamide with substituents selected from alkyl, allyl, phenyl which may be substituted with 1 to 5 substituents selected from alkyl, hydroxyl, alkylhydroxyl, amino, alkylamino and halogen and benzyl which may be substituted with 1 to 5 substituents selected from alkyl, hydroxyl, alkylhydroxyl, amino, alkylamino, halogen and benzyl, urea which may be substituted with alkyl, aminoalkyl or alkylhydroxyl and carbamate which may be substituted with alkyl, aminoalkyl or alkylhydroxyl,

X^{W} signifies a linear spacer of from 1 to 6 atoms length and containing carbon, nitrogen, oxygen and/or sulphur atoms, of which up to 2 atoms can be nitrogen atoms and 1 atom can be oxygen or sulphur,

r^{W} is an integer of from 0 to 1; and

Y^{W} is selected from —CO—, —CS—, —NHCO— and —SO₂—;

or a pharmaceutically acceptable salt thereof.

More embodiments include compounds having the following formula:

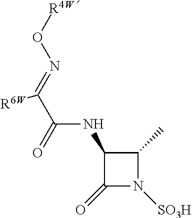

wherein, R^{4W}′ signifies hydrogen, alkyl, C(R^{xW}′)(R^{yW}′)Z^{W}′,

wherein R^{xW}′ and R^{yW}′ are independently selected from hydrogen, alkyl and (C₃-C₆) cycloalkyl; and Z^{W}′ signifies COOH or a group of one of the following two formulae

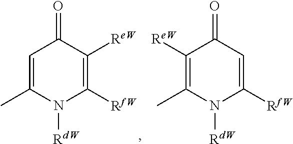

wherein, R^{dW} is hydrogen; amino; monoalkylamino; alkyl; alkoxycarbony; benzyl which may be substituted with 1 to 5 substituents selected from alkyl, hydroxyl, alkylhydroxyl, amino, alkylamino, dialkylamino and halogen, diphenylmethyl; trityl; or ORg whereby R^{gW} is hydrogen, alkyl, benzyl which may be substituted with 1 to 5 substituents selected from alkyl, hydroxyl, alkylhydroxyl, amino, alkylamino and halogen; phenyl which may be substituted with 1 to 5 substituents selected from alkyl, hydroxyl, alkylhydroxyl, amino, alkylamino and halogen;

R^{eW} and R^{fW} are independently selected from hydrogen; alkyl; benzyl which may be substituted with 1 to 5 substituents selected from alkyl, hydroxyl, alkylhydroxyl, amino, alkylamino and halogen; phenyl which may be substituted with 1 to 5 substituents selected from alkyl, hydroxyl, alkylhydroxyl, amino, alkylamino and halogen; OR^{gW} whereby R^{gW} is hydrogen, alkyl, benzyl which may be substituted with 1 to 5 substituents selected from alkyl, hydroxyl, alkylhydroxyl, amino, alkylamino and halogen; phenyl which may be substituted with 1 to 5 substituents selected from alkyl, hydroxyl, alkylhydroxyl, amino, alkylamino and halogen; diphenylmethyl; trityl or alkoxycarbonyl; or, when R^{eW} and R^{fW} are vicinal substituents, R^{eW} and R^{fW} taken together may also be —O—CH═CH—CH₂—, —O—CH₂—CH₂—O—, —CH₂—CH₂—CH₂—, —CH₂—CH₂—CH₂—CH₂—, —CH═CH—CH═CH— or —CH═C(OH)—C(OH)═CH—;

R^{6W}′ signifies phenyl which may be substituted with 1 to 5 substituents selected from alkyl, hydroxyl, alkylhydroxyl, amino, alkylamino, dialkylamino and halogen; or a 5-6 membered heteroaromatic ring which may be substituted with amino, alkyl amino, carbonylamino or halogen.

More embodiments include compounds having the following formula:

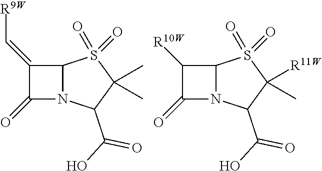

wherein, R^{9W} signifies COOH or a 5-6 membered monocyclic or polycyclic heteroaromatic group;

R^{10W} signifies hydrogen or halogen;

R^{11W} signifies CH₂R^{12W}; CH═CHR^{12W} wherein R^{12W} is hydrogen, halogen, cyano, carboxylic acid, carboxamide which may be substituted, alkoxycarbonyl or a 5-6 membered heteroaromatic ring which is optionally substituted with 1 to 5 substituents selected from alkyl, hydroxyl, alkylhydroxyl, amino, alkylamino, dialkylamino and halogen; or which is optionally fused with a 5-6 membered heteroaromatic ring; CH═NR^{12W}′ wherein R^{12W}′ is amino, alkylamino, dialkylamino, aminocarbonyl, hydroxy, alkylhydroxy,

or a pharmaceutically acceptable salt thereof

More embodiments include compounds having the following formula:

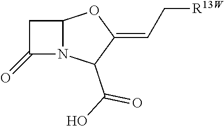

wherein, R^{13W} signifies OR^{14W}; S(O)ₙ^{W}R^{14W} or a 5-6 membered heteroaromatic ring which may be substituted with 1 to 5 substituents selected from alkyl, hydroxyl, alkylhydroxyl, amino, alkylamino, dialkylamino and halogen; whereby n^{W}=0, 1 or 2, and R^{14W} is hydrogen, alkyl, (C₂-C₇) alkene, (C₂-C₇) alkyne or a 5-6 membered heteroaromatic ring which may be substituted with 1 to 5 substituents selected from alkyl, hydroxyl, alkylhydroxyl, amino, alkylamino, dialkylamino and halogen,

or a pharmaceutically acceptable salt thereof

More embodiments include compounds having the following formula:

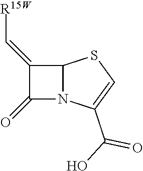

wherein, R^{15W} signifies a 5-6 membered heteroaromatic ring which maybe substituted with 1 to 5 substituents selected from alkyl, hydroxyl, alkylhydroxyl, amino, alkylamino, dialkylamino and halogen; or which is optionally fused with a 5-6 membered heteroaromatic ring and/or which is optionally bound to the exo-methylene group over a —CH═CH— spacer being preferably in the (E)-configuration,

or a pharmaceutically acceptable salt thereof

More embodiments include compounds having the following formula:

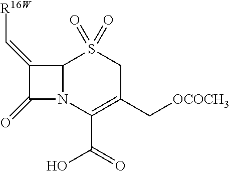

wherein, R^{16W} signifies COOR^{17W}, whereby R^{17W} signifies hydrogen or alkyl; or a 5-6 membered heteroaromatic ring which is optionally fused with a 5-6 membered heteroaromatic ring being optionally substituted with 1 to 5 substituents selected from alkyl, hydroxyl, alkylhydroxyl, amino, alkylamino, dialkylamino, halogen; and/or being optionally bound to the exo-methylene group over a —CH═CH— spacer being preferably in the (E)-configuration,

or a pharmaceutically acceptable salt thereof.

More embodiments include compounds having the following formula:

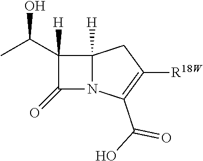

wherein, R^{18W} signifies —S-alkyl —S—(CH₂) 2-NH—CH═NH or a group of one of the following two formulae

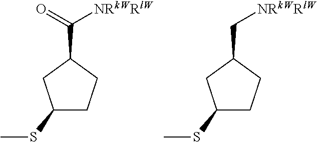

wherein R^{kW} and R^{1W} are individually selected from hydrogen, alkyl, 2-, 3-, 4-carboxyphenyl and sulfamoyl, or a pharmaceutically acceptable salt thereof.

More embodiments include compounds having the following formula:

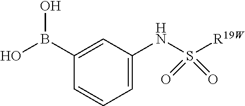

wherein R^{19W} signifies a 5-6 membered heteroaromatic ring which may be substituted with amino, alkylamino, dialkylamino or alkylsulfoxide, or a pharmaceutically acceptable salt thereof.

More embodiments include compounds having the following formula:

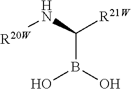

wherein, R^{20W} and R^{21W} are independently selected from a 5-6 membered heteroaromatic ring; phenyl which may be substituted with 1 to 5 substituents selected from alkyl, hydroxyl, alkyl-hydroxyl, amino, alkylamino, dialkylamino and halogen and benzyl which may be substituted with 1 to 5 substituents selected from alkyl, hydroxyl, alkylhydroxyl, amino, alkylamino, dialkylamino and halogen,

or a pharmaceutically acceptable salt thereof

More embodiments include compounds having the following formula:

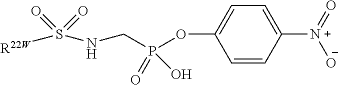

wherein, R^{22W} is selected from a 5-6 membered heteroaromatic ring which may be substituted with 1 to 5 substituents selected from alkyl, hydroxyl, alkylhydroxyl, amino, alkylamino, dialkylamino and halogen and which is optionally fused with a 5-6 membered heteroaromatic ring; phenyl which may be substituted with 1 to 5 substituents selected from alkyl, hydroxyl, alkylhydroxyl, amino, alkylamino, dialkylamino and halogen; and benzyl which may be substituted with 1 to 5 substituents selected from alkyl, hydroxyl, alkylhydroxyl, amino, alkylamino, dialkylamino and halogen.

More embodiments include compounds having the following formula:

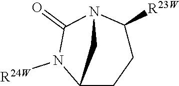

wherein, R^{23W} signifies hydrogen, carboxylic acid, alkoxycarbonyl or carboxamide which may be substituted, and

R^{24W} signifies SO₃H, OSO₃H or OCR^{jW}R^{jW}′COOH, wherein R^{jW} and R^{jW}′ are independently selected from hydrogen, alkyl, phenyl which may be substituted, benzyl which may be substituted, aminoalkyl and alkoxy.

Some β-lactamase inhibitors useful with the methods, compositions and compounds provided herein include compound SYN-2190, having the following formula:

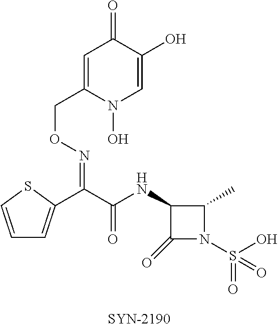 [SMILES: C[C@H]1[C@H](NC(=O)/C(=N/OCc2cc(=O)c(O)cn2O)c2cccs2)C(=O)N1S(=O)(=O)O]

Some β-lactamase inhibitors useful with the methods, compositions and compounds provided herein include compound BLI-489, having the following formula:

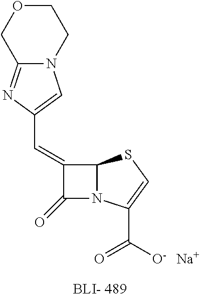 [SMILES: O=C([O-])C1=CS[C@@H]2/C(=C\c3cn4c(n3)COCC4)C(=O)N12 ; [Na+]]

Some β-lactamase inhibitors useful with the methods, compositions and compounds provided herein include compound AM-112, having the following formula:

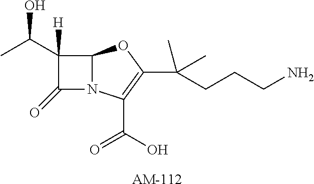

The following examples of specific β-lactamase inhibitors are used for the purposes of illustration only, and should not be considered as limiting.

### EXAMPLES

### General Procedures

Materials used in preparing the cyclic boronic acid derivatives described herein may be made by known methods or are commercially available. It will be apparent to the skilled artisan that methods for preparing precursors and functionality related to the compounds claimed herein are generally described in the literature including, for example, procedures described in U.S. Pat. No. 7,271,186 and WO2009064414, each of which is incorporated by reference in its entirety. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

(¹H) nuclear magnetic resonance spectra (NMR) were measured in the indicated solvents on either a Broker NMR spectrometer (Avance™ DRX500, 500 MHz for 1H) or Varian NMR spectrometer (Mercury 400BB, 400 MHz for 1H). Peak positions are expressed in parts per million (ppm) downfield from tetramethylsilane. The peak multiplicities are denoted as follows, s, singlet; d, doublet; t, triplet; q, quartet; quin, quintet; sex, sextet; sep, septet; non, nonet; dd, doublet of doublets; td, triplet of doublets; m, multiplet.

The following abbreviations have the indicated meanings:
n-BuLi=n-butyllithium
t-Bu=tert-butyl
DCM=dichloromethane
DMF=N,N-dimethylformamide
DIPEA=diisopropylethylamine
EDCI=1-ethyl-3-(3-dimethylaminopropyl) carbodiimide
ESBL=extended-spectrum β-lactamase
ESIMS=electron spray mass spectrometry
EtOAc=ethyl acetate
EtOH=ethanol
HATU=2-(7-aza-1H-benzotriazole-1-yl)-1,1,3,3-tetramethyluronium hexafluorophosphate
HCl=hydrochloric acid
HOBt=hydroxybenzotriazole
Im=imidazole
LiHMDS=lithium bis(trimethylsilyl)amide
MeCN=acetonitrile
NaHCO₃ sodium bicarbonate
Na₂SO₄=sodium sulfate
NMM=N-methylmorpholine
NMR=nuclear magnetic resonance
Pd/C=palladium on carbon
TBDMSCl=tert-butyldimethylsilyl chloride
TBS=tert-butyldimethylsilyl
TFA=trifluoroacetic acid
THF=tetrahydrofuran
TLC=thin layer chromatography
TMS=trimethylsilyl
TPPB=tris(pentafluorophenyl)borane monohydrate

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Formula (I)

Compounds of formula I where R¹ is an acylamino group and X is a carboxylic acid can be prepared as depicted in Scheme 1.

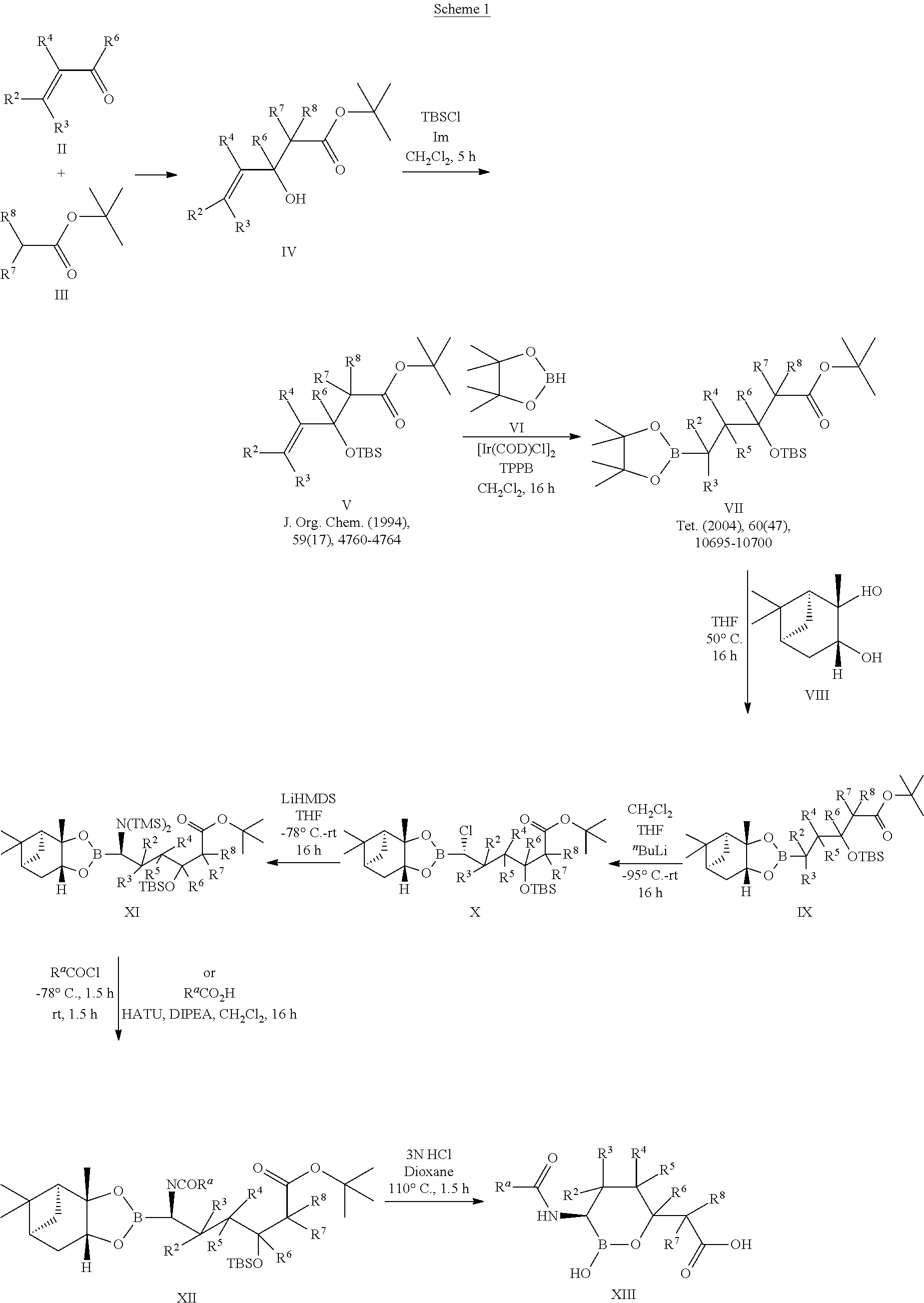

The addition of enolates to substituted α,β-unsaturated ketones or aldehydes to form β-hydroxy esters is a well-known reaction (Scheme 1). Substituents R⁷ and R⁸ of formula I may be controlled by use of the appropriate α-mono or di-substituted ester III. Similarly, substituents R², R³, and R⁴ may be controlled by use of the appropriate substituted substituted α,β-unsaturated ketones or aldehydes analog II. Precursors of structure IV, where R⁶ and R⁷ or R⁸ are combined together, may be made following the known procedures [J. Am. Chem. Soc. (1982), 104, 1735-7, Tetrahedron Lett. (2003), 44, 1259-62]. The β-hydroxy ester of structure IV is protected with an acid-sensitive protecting group, affording V; this selection allows simultaneous deprotection of the boronate ester and hydroxyl protecting group in the final step, resulting in a cyclized product. The pinacol boronate VII is formed from substituted V using iridium catalysis [Tetrahedron (2004), 60, 10695-700]. Trans-esterification was readily achieved with optically active pinane diol VIII to result in IX [Tetrahedron: Asymmetry, (1997), 8, 1435-40]. Transesterification may also be achieved from the catechol ester analog of VII. Such catechol esters can be made by reaction of V with commercially available catechol borane [Tetrahedron (1989), 45, 1859-85]. Homologation of IX to give chloromethylene addition product X with good stereocontrol may be achieved via Matteson reaction conditions (WO0946098). The chloro derivative X can be utilized to introduce a substituted amine group at the C3-position of the oxaborinane-2-ol. Stereospecific substitution with hexamethyldisilazane gives the corresponding bis(trimethylsilyl) amide XI which may be reacted in situ with an acid chloride to result directly in analogs of structure XII. Such analogs of XII can also be made via coupling of the bis-TMS amine with commercially available carboxylic acids under typical amide coupling conditions (e.g., carbodiimide or HATU coupling). Simultaneous deprotection of the pinane ester, the tert-butyldimethylsilyloxy group and the tert-butyl ester group and concomitant cyclization are achieved by heating with dilute HCl, affording the desired oxaborinane derivatives of structure XIII. This transformation may also be achieved by treatment with BCl₃ or BBr₃. Alternatively, the deprotection may be attained via trans-esterification with isobutyl boronic acid in presence of dilute HCl (WO09064413).

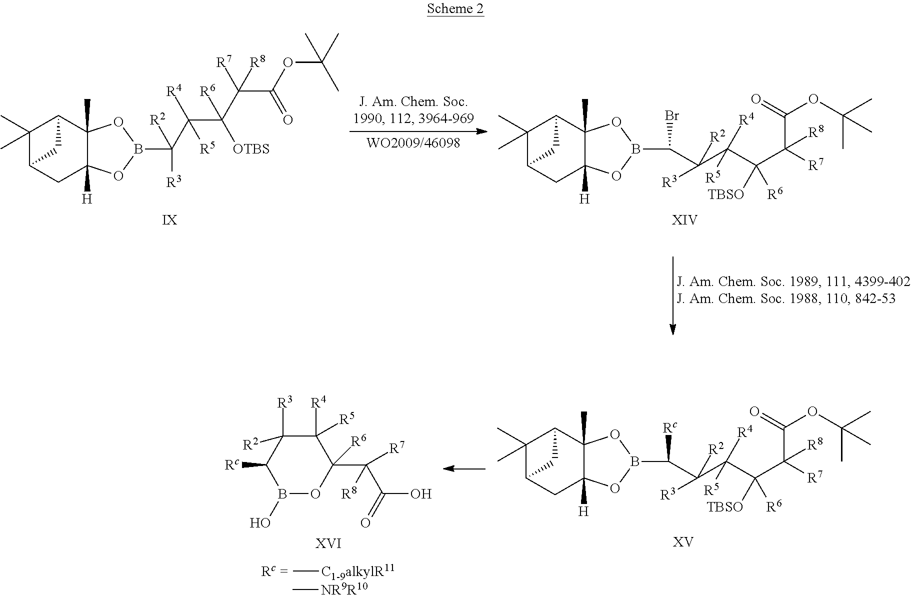

Compounds of structure XVI where R¹ of Formula I is an alkyl, aralkyl or aminoaryl group may be made from bromo intermediate XIV as shown in Scheme 2 [J. Organomet. Chem. (1992), 431, 255-70]. Such bromo derivatives may be made as analogously to the chloro compounds of Scheme 1, utilizing dibromomethane [J. Am. Chem. Soc. (1990), 112, 3964-969]. Displacement of the bromo group in XIV can be achieved by α-alkoxy substituted alkyllithium agents [J. Am. Chem. Soc. (1989), 111, 4399-402; J. Am. Chem. Soc. (1988), 110, 842-53] or organomagnesium reagents (WO0946098) or by the sodium salt of alkyl or aryl carbamate derivatives [J. Org. Chem. (1996), 61, 7951-54], resulting in XV. Cyclization of XV to afford XVI may be achieved under the conditions described in Scheme 1.

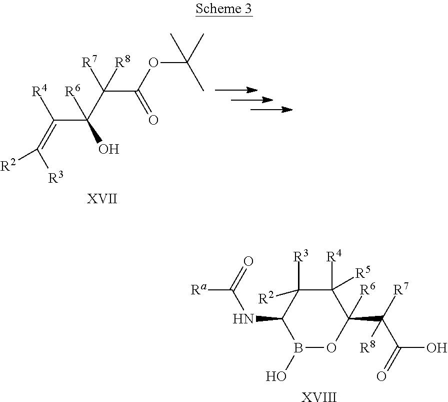

Compounds of formula XIII and XVI are mixtures of 3,6-cis- and 3,6-trans-isomers. These analogs can be made in enantiomerically pure form as single isomers by starting (as in Scheme 1) with a single enantiomer (XVII), as shown in Scheme 3. A variety of methods to prepare such enantiomerically pure β-hydroxy esters are known in literature, for example via resolution [Org. Lett., (2008), 10, 3907-09] or stereoselective synthesis [Tetrahedron, (2000), 56, 917-47]. Such single isomers result in enantiomerically pure cis-compounds XIII or XVI when used in the sequences depicted in Schemes 1 and 2.

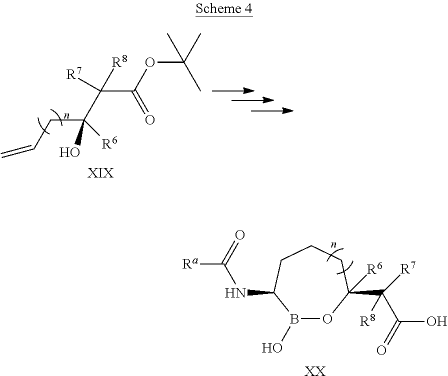

The sequence shown in Scheme 1 also allows for varied ring sizes in formula I such as 7- and 8-membered rings. For example, a seven-membered analog XX where n=1 can be achieved by using the corresponding allyl intermediate (XIX) as a starting material (Scheme 4). Such allyl derivatives as XIX can be made utilizing one of several well known β-hydroxy ester preparations [Tetrahedron (2007), 63, 8336-50]. Intermediate XIX where n=2 can be prepared as described in Scheme 1 to give corresponding 8-membered compound of structure XX starting from pent-4-ene-1-al [J. Med. Chem. (1998), 41(6), 965-972].

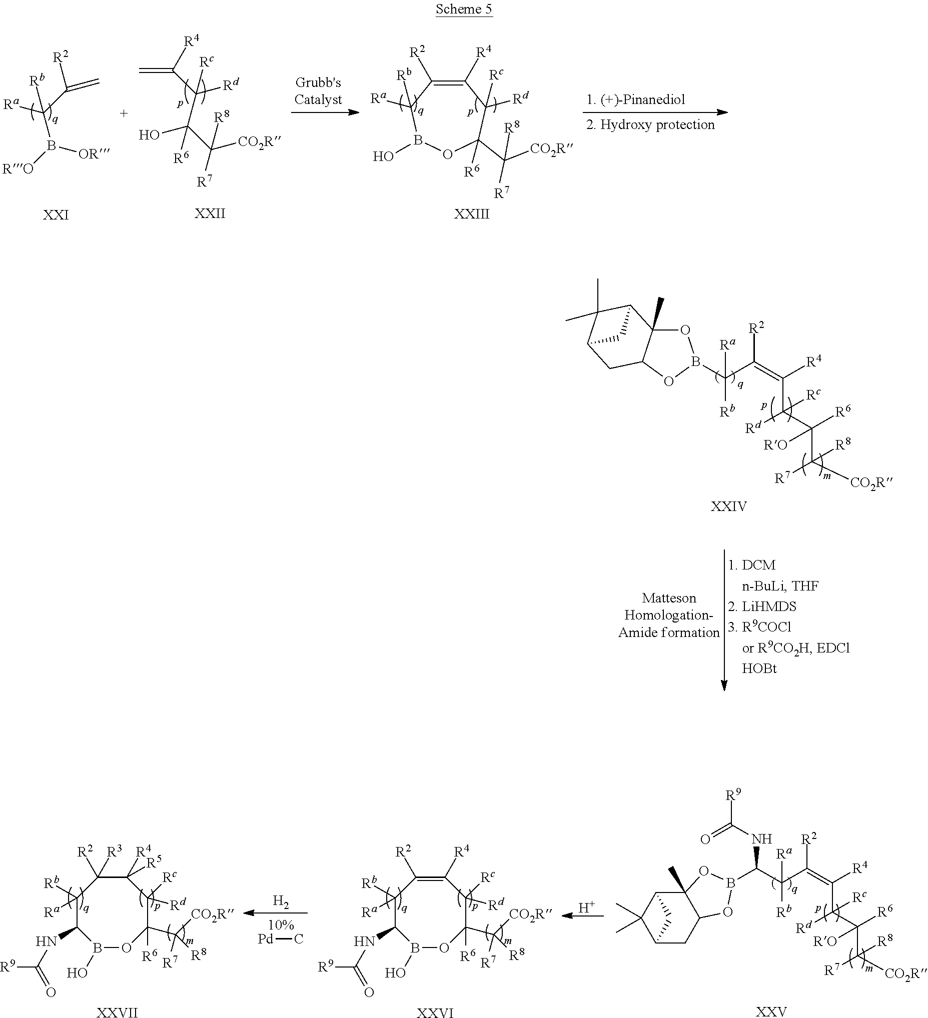

Compounds of formula XXVI and XXVII can be made following the sequence depicted in Scheme 5. Ring-Closing Metathesis reaction with boronated olefins (XXI) and olefin substituted β-hydroxy esters (XXII) result in cyclic boronates of formula XXIII Such cyclic boronates (XXIII) undergo ready esterification with (+)-pinane diol to give required Matteson reaction precursors upon protection of the resulting alcohol with groups such as t-butyldimethylsilyl- or benzyl or trityl. Matteson homologation followed by amide formation result in compounds of formula XXV with high stereoselectivity, as described above. Acid mediated hydrolysis of compounds of XXV upon deprotection give cyclic boronate (XXVI). Double bond substitution of XXVI can be further modified to other analogs such as saturated cyclic boronate (XXVII) by catalytic hydrogenation. The above sequence can be utilized to make 7- or 8-membered rings with double bond at a desired position by varying p and q of XXI and XXII.

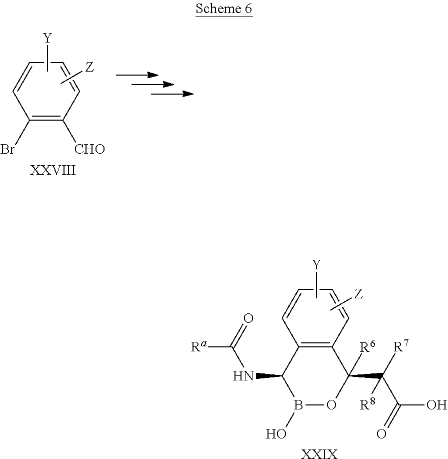

Compounds of formula I where R² and R⁴ taken together form an aryl ring can be made from commercially available substituted aryl precursors as XXVIII. Substitution of the bromine atom by a boronate ester may be done under palladium catalyzed conditions [Tetrahedron (2002), 58, 9633-95]. The steps of hydroxy ester formation, α-amidoboronate preparation and cyclization can be attained by synthetic steps analogous to those in Scheme 1 to give compounds XXIX.

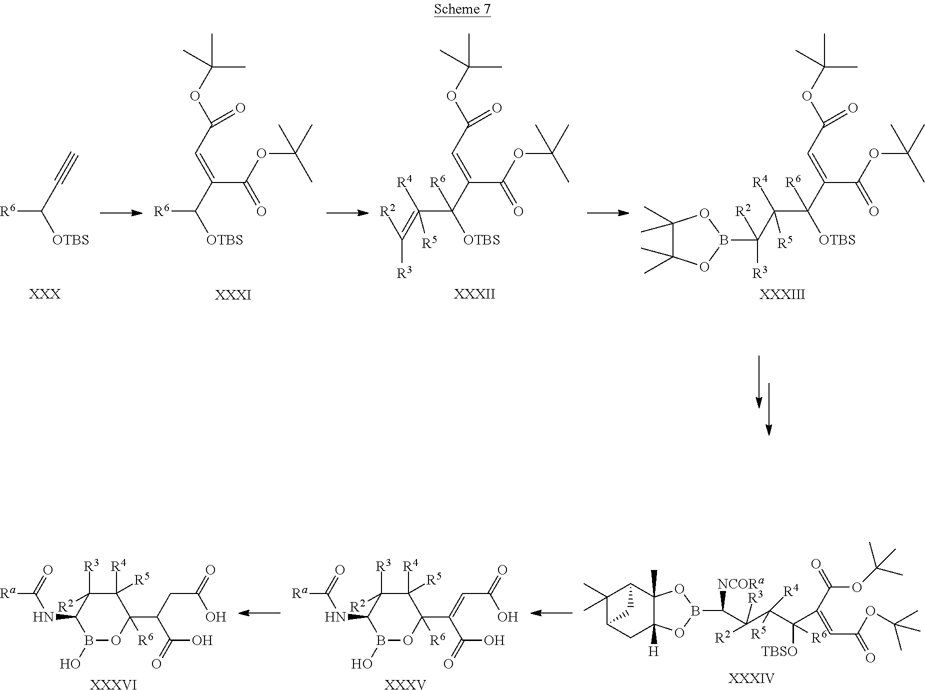

Compounds of formula I where R⁷ and R⁸ are substituted as maleate (XXXV) or succinate (XXXVI) may be made following the sequence shown in Scheme 7. Maleate intermediates such as XXXII can be transformed to analogs XXXV analogously to the steps in Scheme 1. Analogs of XXXV can be further transformed to the corresponding succinic acids of structure XXXVI by catalytic hydrogenation. Maleate intermediate XXXII may be assembled from intermediate XXXI by successive deprotection of the TBS group, oxidation to the aldehyde, addition of vinyl Grignard and reprotection as a TBS ether. Intermediate XXXI may be formed from a protected propargylic alcohol XXX following methods known in the literature [Tetrahedron, (2002), 58, 6545-54].

### Illustrative Compound Examples

Synthesis of 2-((3R)-2-hydroxy-3-(2-(thiophen-2-yl)acetamido)-1,2-oxaborinan-6-yl)acetic acid. An example synthesis of 1 is depicted in Scheme 8 and Example 1.

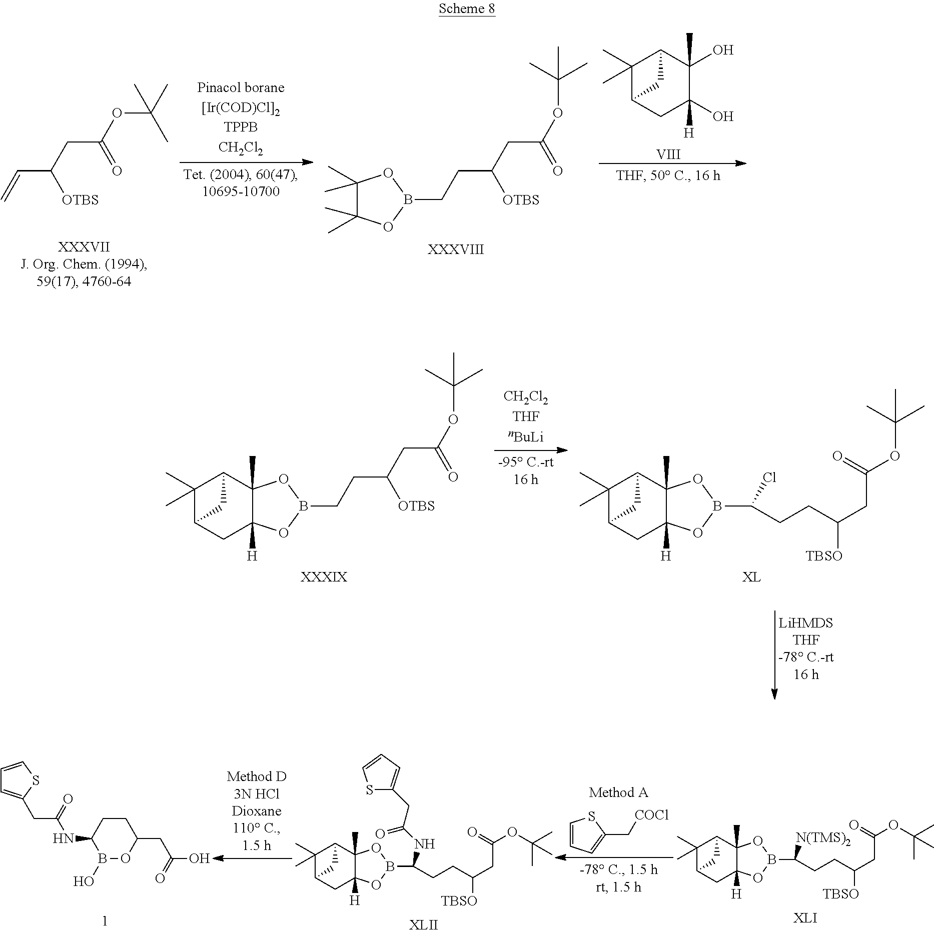

### Example 1

### Step 1

A round-bottom flask charged with [Ir(cod)Cl]₂ (350 mg, 0.52 mmol) and 1,4-bis(diphenylphosphanyl)butane (446 mg, 1.04 mmol) was flushed with argon. DCM (60 mL), pinacolborane (3 mL, 21 mmol) and tert-butyl-3-(tert-butyldimethylsilyloxy)pent-4-enoate XXXVII [J. Org. Chem., (1994), 59(17), 4760-4764] (5 g, 17.48 mmol) in 5 mL of DCM were added successively at room temperature. The mixture was then stirred at room temperature for 16 h. The reaction was quenched with MeOH (3 mL) and water (10 mL), the product was extracted with ether, and dried. Chromatography on silica gel (100% DCM→50% EtOAc/DCM gave tert-butyl 3-(tert-butyl dimethylsilyloxy)-5-(4,4,5,5-tetramethyl-1,3,2-dioxaborolan-2-yl)pentanoate XXXVIII (5.5 g, 13.2 mmol, 75.5% yield).

### Step 2

To a solution of tert-butyl 3-(tert-butyldimethylsilyloxy)-5-(4,4,5,5-tetramethyl-1,3,2-dioxaborolan-2-yl)pentanoate XXXVIII (5.4 g, 13 mmol) in THF (25 mL) was added (1 S,2S,3R,5S)-2,6,6-trimethylbicyclo[3.1.1]heptane-2,3-diol (2.4 g, 14.3 mol) at room temperature. The reaction mixture was stirred for 16 h and then was concentrated under vacuum. The residue was purified by column chromatography (100% hexane→40% EtOAc/hexane) on silica gel to give 1-(tert-butoxy)-3-[(tert-butyldimethylsilyl)oxy]-1-oxo-6-[(2S,6R)-2,9,9-trimethyl-3,5-dioxa-4-boratricyclo[6.1.1.02,6]decan-4-yl]hexan-3-yl XXXIX (5.5 g, 11 mmol, 84.6% yield).

### Step 3

To a solution of DCM (1.5 mL, 23.6 mmol) in THF (30 mL) at −100° C. was added 2.5 M n-butyl lithium in hexane (5.19 mL, 12.98 mmol) slowly under nitrogen and down the inside wall of the flask whilst maintaining the temperature below −90° C. The resulting white precipitate was stirred for 30 minutes before the addition of 1-(tert-butoxy)-3-[(tert-butyldimethylsilyl)oxy]-1-oxo-6-[(2S,6R)-2,9,9-trimethyl-3,5-dioxa-4-boratricyclo [6.1.1.02,6]decan-4-yl]hexan-3-yl XXXIX (5.5 g, 11 mmol) in THF (10 mL) at −90° C. Zinc chloride (23.6 mL, 0.5 M in diethyl ether, 11.86 mmol) was then added to the reaction mixture at −90° C. and then the reaction was allowed to warm to room temperature where it was stirred for 16 h. The reaction was quenched with a saturated solution of ammonium chloride and the phases were separated. The aqueous phase was then extracted with diethyl ether (3×50 mL) and the combined organic extracts were dried over Na₂SO₄, filtered and concentrated under reduced pressure. The concentrated material was then chromatographed (100% hexane→50% EtOAc/hexane) to obtain 6-(tert-butoxy)-4-[(tert-butyldimethylsilyl)oxy]-1-chloro-6-oxo-1-[(2S,6R)-2,9,9-trimethyl-3,5-dioxa-4-boratricyclo[6.1.1.02,6]decan-4-yl]hexyl XL (5.6 g, 10.5 mmol, 95.4% yield).

### Step 4-5

Chloro intermediate XL (1.2 g, 2.33 mmol) in THF (10 mL) was cooled to −78° C. under nitrogen. A solution of LiHMDS (2.33 mL, 1.0 M in THF, 2.33 mmol) was added slowly and the reaction flask was then allowed to warm to room temperature where it was stirred for 16 h. Method A: The resulting was cooled to −78° C. and 5-thiopheneacetyl chloride was added and the solution stirred at −78° C. for 1.5 h. Then, the cooling bath was removed and the solution stirred at ambient temperature for 1.5 h. The reaction was quenched with water and extracted twice with EtOAc. The organic layers were combined, washed with water, brine, dried (Na₂SO₄) and concentrated in vacuo to afford a pale yellow solid as crude product. The residue was chromatographed on a silica column (100% DCM→40% EtOAc/DCM) to afford 570 mg of 6-(tert-butoxy)-4-[(tert-butyldimethylsilyl)oxy]-6-oxo-1-(thiophen-2-ylacetamido)-1-[(2S,6R)-2,9,9-trimethyl-3,5-dioxa-4-boratricyclo[6.1.1.02,6]decan-4-yl]hexylidyne XLII as a white solid (570 mg, 0.92 mmol, 39.5% yield).

### Step 6

### Method D

To a solution of amide XLII (250 mg, 0.40 mmol) in 1,4-dioxane (10 mL) was added 10 mL of 3 N HCl. The mixture was heated to 110° C. for 90 min. The solution was cooled and diluted with 10 mL of water and extracted twice with 10 mL of diethyl ether. The aqueous layer was concentrated to afford a sticky residue as crude product. The residue was rinsed with 5 mL of water, dissolved in 10% MeCN-water and lyophilized to afford 2-((3R)-2-hydroxy-3-(2-(thiophen-2-yl)acetamido)-1,2-oxaborinan-6-yl)acetic acid 1 as white powder (100 mg, 0.337 mmol, 84.1% yield). ¹H NMR (CD₃OD) δ ppm 0.94-1.35 (m, 1H), 1.35-1.54 (m, 1H), 1.54-1.68 (m, 1H), 1.68-2.00 (m, 1H), 2.20-2.67 (m, 3H), 3.93 (s, 1H), 3.98 (s, 1H), 4.02-4.23 (m, 2H), 6.98-7.05 (m, 2H), 7.32-7.36 (m, 1H); ESIMS found for C₁₂H₁₆BNO₅S m/z 280 (100%) (M−H₂O)+.

Alternative procedures for Steps 5 and 6 are shown in Scheme 9.

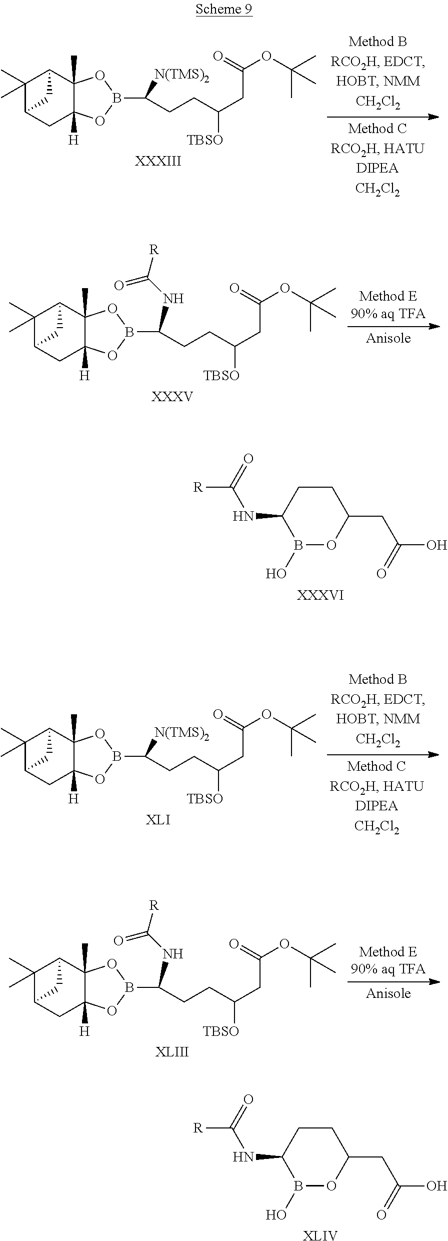

### Step 5, Method B

To a solution of the acid (0.36 mmol) in DCM (10 mL) at 0° C. under nitrogen was added EDCI (86 mg, 0.45 mmol) and HOBT (48 mg, 0.36 mmol). After stirring at 0° C. for 30 minutes, a solution of the bis-silyl amide intermediate XLI (0.3 mmol) in DCM (2 mL) followed by N-methyl-morpholine (65 μL, 0.6 mmol) were sequentially added at 0° C. The reaction flask was then allowed to warm to room temperature. After stirring at room temperature overnight, the reaction mixture was washed with water, then brine, dried (Na₂SO₄), filtered and concentrated under vacuum. The residue was purified by column chromatography to produce intermediate XLIII.

### Step 5, Method C

A solution of bis-silyl amide XLI (0.5 mmol) and acid in dry DCM (10 mL) were cooled to 0° C. Then DIPEA (1.5 mmol) was added drop wise followed HATU (0.75 mmol). The mixture was then allowed to warm to room temperature. After TLC has indicated complete conversion (˜3 h) of the starting materials, the reaction was diluted with additional DCM (20 mL). The reaction mixture was washed with water (3×5 mL), brine (10 mL), and dried over Na₂SO₄. After removal of the solvent, the residue was subjected to flash column chromatography to produce intermediate XLIII.

### Step 6, Method E

To a solution of amide (XLIII) (0.1 mmol) in dichloroethane (2 mL) at 0° C. was treated with pre-cooled 90% aq. TFA (4 mL) and stirred at room temperature for 3 hrs. The reaction mixture was evaporated in vacuo, azeotroped with MeCN (3×5 mL) and the residue was triturated with ether (5 mL). The product separated was filtered, dissolved in dioxane-water mixture and freeze dried to give the final product XLIV as a fluffy solid.

The following compounds are prepared in accordance with the procedure described in the above Example 1 using methods A and D.

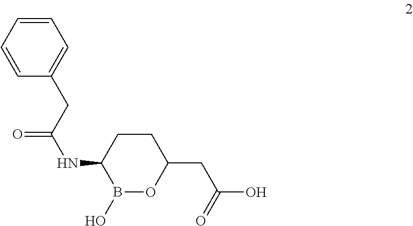 [SMILES: O=C(O)CC1CC[C@H](NC(=O)Cc2ccccc2)B(O)O1]

2-((3R)-2-hydroxy-3-(2-phenylacetamido)-1,2-oxaborinan-6-yl)acetic acid 2. ¹H NMR (CD₃OD) δ ppm 0.82-1.33 (m, 1H), 1.33-1.51 (m, 1H), 1.51-168 (m, 1H), 1.69-2.00 (m, 1H), 2.14-2.34 (m, 1H), 2.34-2.69 (m, 2H), 3.74-3.76 (m, 2H), 3.98-4.20 (m, 1H), 7.22-7.41 (m, 5H); ESIMS found for C14H18BNO5 m/z 274 (100%) (M−H₂O)⁺.

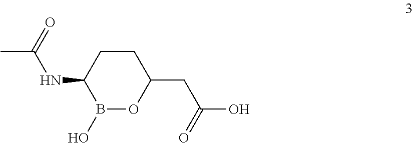

2-((3R)-3-acetamido-2-hydroxy-1,2-oxaborinan-6-yl)acetic acid 3. ¹H NMR (CD₃OD) δ ppm 1.07-1.36 (m, 1H), 1.36-159 (m, 1H), 1.59-1.73 (m, 1H), 173-2.09 (m, 1H), 2.15-2.16 (d, 3H), 2.35-2.69 (m, 3H), 4.01-4.23 (m, 1H); ESIMS found for C₈H₁₄BNO₅ m/z 198 (100%) (M−H₂O)⁺.

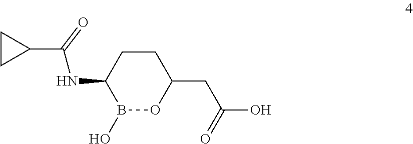

2-((3R)-3-(cyclopropanecarboxamido)-2-hydroxy-1,2-oxaborinan-6-yl)acetic acid 4. ¹H NMR (CD₃OD) δ ppm 0.98-1.32 (m, 5H), 1.32-1.67 (m, 2H), 1.67-2.06 (m, 2H), 2.27-2.66 (m, 3H), 3.98-4.16 (m, 1H); ESIMS found for C₁₀H₁₆BNO₅ m/z 224 (100%) (M−H₂O)⁺.

The following compounds are prepared starting from enantiomerically pure (R)-tert-butyl 3-hydroxypent-4-enoate (J. Am. Chem. Soc. 2007, 129, 4175-4177) in accordance with the procedure described in the above Example 1.

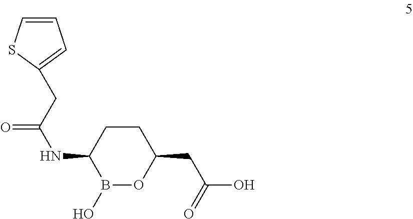 [SMILES: O=C(O)C[C@@H]1CC[C@H](NC(=O)Cc2cccs2)B(O)O1]

2-((3R,6S)-2-hydroxy-3-(2-(thiophen-2-yl)acetamido)-1,2-oxaborinan-6-yl)acetic acid 5. ¹H NMR (CD₃OD) δ ppm 0.97-1.11 (q, 1H), 1.47-1.69 (m, 2H), 1.69-1.80 (m, 1H), 2.21-2.33 (td, 1H), 2.33-2.41 (dd, 1H), 2.58-2.67 (m, 1H), 3.97 (s, 2H), 4.06-4.14 (m, 1H), 6.97-7.04 (m, 1H), 7.04-7.08 (m, 1H), 7.34-7.38 (dd, 1H); ESIMS found for C₁₂H₁₆BNO₅S m/z 280 (100%) (M−H₂O)⁺.

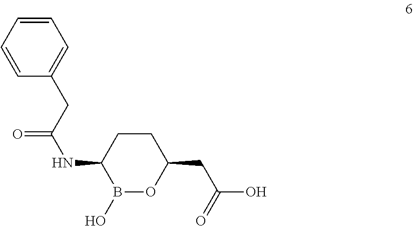 [SMILES: O=C(O)C[C@@H]1CC[C@H](NC(=O)Cc2ccccc2)B(O)O1]

2-((3R,6S)-2-hydroxy-3-(2-phenylacetamido)-1,2-oxaborinan-6-yl)acetic acid 6. ¹H NMR (CD₃OD) δ ppm 0.86-1.02 (m, 1H), 1.44-1.53 (dd, 1H), 1.53-1.66 (td, 1H), 1.68-1.78 (m, 1H), 2.17-2.26 (dd, 1H), 2.26-2.36 (dd, 2H), 3.75 (s, 2H), 4.02-4.12 (m, 1H), 7.22-7.40 (m, 5H); ESIMS found for C₁₄H₁₈BNO₅ m/z 274 (100%) (M−H₂O)⁺.

The following compounds are prepared in accordance with the procedure described in the above Example 1 starting from enantiomerically pure (R)-tert-butyl 3-hydroxypent-4-enoate (J. Am. Chem. Soc. 2007, 129, 4175-4177) using methods B and D.

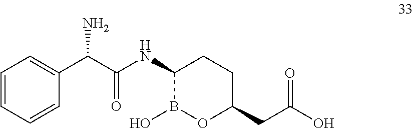

2-((3R,6S)-3-((S)-2-amino-2-phenylacetamido)-2-hydroxy-1,2-oxaborinan-6-yl)acetic acid 33 was isolated as the HCl salt. ¹H NMR (CD₃OD) δ ppm 1.24-1.27 (m, 1H), 151-1.72 (m, 3H), 2.45-2.50 (dd, J=5 Hz, J=5 Hz, 1H), 2.55-2.63 (dd, J=2 Hz, J=3 Hz, 1H), 3.66-3.71 (m, 1H), 4.38-4.53 (m, 1H), 4.99-5.09 (d, 1H), 7.48-7.56 (m, 5H); ESIMS found for C₁₄H₁₉BN₂O₅ m/z 289 (M−H₂O)⁺.

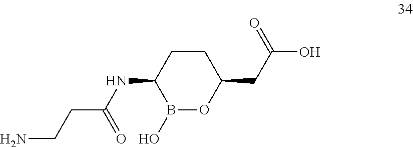 [SMILES: NCCC(=O)N[C@H]1CC[C@@H](CC(=O)O)OB1O]

2-((3R,6S)-3-(3-aminopropanamido)-2-hydroxy-1,2-oxaborinan-6-yl)acetic acid 34 was isolated as the HCl salt. ¹H NMR (CD₃OD) δ ppm 1.24-1.29 (td, J=13 Hz. J=3 Hz, 1H), 1.55-1.62 (td, J=14 Hz, J=4 Hz, 1H), 1.68-1.72 (m, 1H), 1.79-1.82 (m, 1H), 2.43-2.47 (dd, J=6 Hz, J=6 Hz, 2H), 2.70-2.74 (m, 2H), 2.83-2.86 (t, J=7 Hz, 2H), 3.26-3.29 (t, J=7 Hz, 1H), 4.10-4.16 (m, 1H); ESIMS found for C₉H₁₇BN₂O₅ m/z 227 (M−H₂O)⁺.

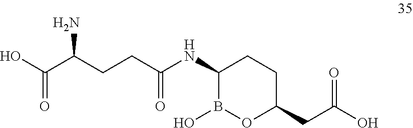 [SMILES: N[C@@H](CCC(=O)N[C@H]1CC[C@@H](CC(=O)O)OB1O)C(=O)O]

(S)-2-amino-5-((3R,6S)-6-(carboxymethyl)-2-hydroxy-1,2-oxaborinan-3-ylamino)-5-oxopentanoic acid 35 was isolated as the HCl salt. ¹H NMR (CD₃OD) δ ppm 150-1.66 (m, 2H), 1.66-1.84 (m, 2H), 2.10-2.20 (sex, J=8 Hz 1H), 2.20-2.29 (m, 1H), 2.40-2.47 (m, 2H), 2.55-2.59 (q, J=7 Hz 1H), 2.69-2.75 (m, 1H), 2.94-2.98 (td, J=9 Hz, J=2 Hz 1H), 3.99-4.12 (m, 2H); ESIMS found for C₁₁H₁₉BN₂O₇ m/z 302.8 (M+H).

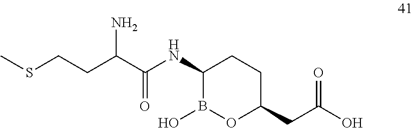 [SMILES: CSCCC(N)C(=O)N[C@H]1CC[C@@H](CC(=O)O)OB1O]

2-((3R,6S)-3-(2-amino-4-(methylthio)butanamido)-2-hydroxy-1,2-oxaborinan-6-yl)acetic acid 41 was isolated as the HCl salt. ¹H NMR (CD₃OD) δ ppm 1.45-1.65 (m, 1H), 1.65-175 (m, 1H), 1.75-186 (m, 1H), 1.86-2.05 (m, 1H), 2.09-2.20 (m, 4H), 2.46-2.73 (m, 6H), 2.84-2.86 (t, J=6 Hz, 1H), 3.99-4.02 (t, J=7 Hz, 1H), 4.38-4.46 (m, 1H); ESIMS found for C₁₁H₂₁BN₂O₅S m/z 287 (M−H₂O)⁺.

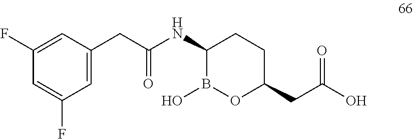 [SMILES: O=C(O)C[C@@H]1CC[C@H](NC(=O)Cc2cc(F)cc(F)c2)B(O)O1]

2-((3R,6S)-3-(2-(3,5-difluorophenyl)acetamido)-2-hydroxy-1,2-oxaborinan-6-yl)acetic acid 66 was isolated as the HCl salt. ¹H NMR (CD₃OD) δ ppm 0.98-1.07 (q, J=13 Hz, 1H), 1.55-168 (m, 2H), 1.73-1.79 (dd, J=6 Hz, J=3 Hz, 1H), 2.22-2.26 (dd, J=15 Hz, J=6 Hz, 1H), 2.33-2.38 (dd, J=13 Hz, J=7 Hz, 1H), 2.62-2.63 (m, 1H), 3.78 (s, 2H), 4.05-4.12 (m, 1H), 6.88-5.93 (tt, J=5 Hz, J=2 Hz, 1H), 6.97-7.01 (dd, J=5 Hz, J=2 Hz, 2H); ESIMS found for C₁₄H₁₆BF₂NO₅ m/z 310.1 (M−H₂O)⁺.

The following compounds are prepared in accordance with the procedure described in the above Example 1 starting from enantiomerically pure (R)-tert-butyl 3-hydroxypent-4-enoate (J. Am. Chem. Soc. 2007, 129, 4175-4177) using methods A and E.

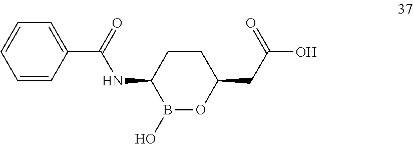 [SMILES: O=C(O)C[C@@H]1CC[C@H](NC(=O)c2ccccc2)B(O)O1]

2-((3R,6S)-3-benzamido-2-hydroxy-1,2-oxaborinan-6-yl)acetic acid 37. ¹H NMR (CD₃OD) δ ppm 1.10-1.19 (q, J=11 Hz, 1H), 1.60-1.65 (dd, J=14 Hz, J=3 Hz, 1H), 1.71-1.80 (td, J=9 Hz, J=3 Hz, 1H), 1.91-1.96 (d, J=14 Hz, 1H), 2.32-2.38 (dd, J=15 Hz, J=6 Hz, 1H), 2.44-2.49 (dd, J=15 Hz, J=7 Hz, 1H), 2.82-2.84 (d, J=4 Hz, 1H), 4.10-4.17 (m, 1H), 7.57-7.60 (t, J=8 Hz, 2H), 7.70-7.73 (t, J=8 Hz, 1H), 8.00-8.02 (d, J=8 Hz 2H); ESIMS found for C₁₃H₁₆BNO₅ m/z 260 (M−H₂O)⁺.

The following compounds are prepared in accordance with the procedure described in the above Example 1 starting from enantiomerically pure (R)-tert-butyl 3-hydroxypent-4-enoate (J. Am. Chem. Soc. 2007, 129, 4175-4177) using methods B and E.

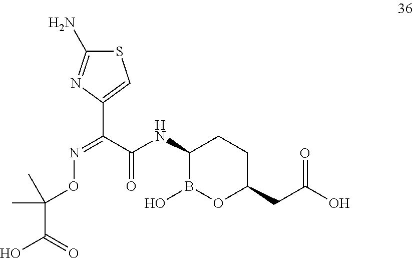 [SMILES: CC(C)(O/N=C(\C(=O)N[C@H]1CC[C@@H](CC(=O)O)OB1O)c1csc(N)n1)C(=O)O]

2-((Z)-1-(2-aminothiazol-4-yl)-2-((3R,6S)-6-(carboxymethyl)-2-hydroxy-1,2-oxaborinan-3-ylamino)-2-oxoethylideneaminooxy)-2-methylpropanoic acid 36 was isolated as the TFA salt. ¹H NMR (CD₃OD) δ ppm 1.60 (s, 3H), 161 (s, 3H), 1.62-1.75 (m, 2H), 1.77-1.82 (m, 1H), 1.86-1.91 (m, 1H), 2.55-2.58 (t, J=6 Hz, 2H), 2.90-2.94 (t, J=6 Hz, 2H), 4.37-4.42 (m, 1H), 7.11 (s, 1H); ESIMS found for C₁₅H₂₁BN₄O₈S m/z 411 (M−H₂O)⁺.

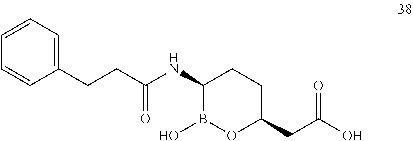 [SMILES: O=C(O)C[C@@H]1CC[C@H](NC(=O)CCc2ccccc2)B(O)O1]

2-((3R,6S)-2-hydroxy-3-(3-phenylpropanamido)-1,2-oxaborinan-6-yl)acetic acid 38. ¹H NMR (CD₃OD) δ ppm 0.78-0.87 (q, J=13 Hz, 1H), 1.40-1.46 (dd, J=10 Hz, J=3 Hz, 1H), 1.54-1.62 (dt, J=8 Hz, J=4 Hz, 1H), 1.63-1.70 (d, J=13 Hz, 1H), 2.24-2.29 (dd, J=15 Hz, J=6 Hz, 1H), 2.36-2.40 (dd, J=8 Hz, J=3 Hz, 1H), 2.53-2.56 (d, J=3.2 Hz, 1H), 2.74-2.78 (t, J=7 Hz, 2H), 2.98-3.01 (t, J=6 Hz, 2H), 3.90-4.03 (m, 1H), 7.18-7.23 (m, 1H), 7.25-7.33 (m, 4H); ESIMS found for C₁₅H₂₀BNO₅ m/z 288 (M−H₂O)⁺.

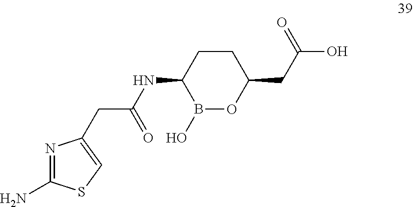 [SMILES: Nc1nc(CC(=O)N[C@H]2CC[C@@H](CC(=O)O)OB2O)cs1]

2-((3R,6S)-3-(2-(2-aminothiazol-4-yl)acetamido)-2-hydroxy-1,2-oxaborinan-6-yl)acetic acid 39 was isolated as the TFA salt. ¹H NMR (CD₃OD) δ ppm 1.25-1.36 (m, 1H), 163-1.76 (m, 3H), 2.40-2.43 (d, J=6 Hz 2H), 2.68-2.70 (m, 1H), 3.72 (s, 2H), 4.17-4.21 (m, 1H), 6.69 (s, 1H); ESIMS found for C₁₁H₁₆BN₃O₅S m/z 296.1 (M−H₂O)⁺.

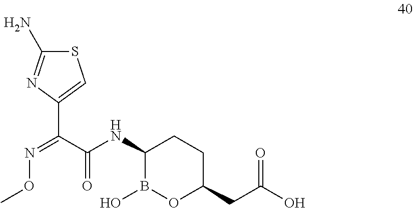 [SMILES: CO/N=C(\C(=O)N[C@H]1CC[C@@H](CC(=O)O)OB1O)c1csc(N)n1]

2-((3R,6S)-3-((Z)-2-(2-aminothiazol-4-yl)-2-(methoxyimino)acetamido)-2-hydroxy-1,2-oxaborinan-6-yl)acetic acid 40 was isolated as the TFA salt. ¹H NMR (CD₃OD) δ ppm 1.56-1.67 (m, 2H), 1.76-1.81 (m, 1H), 1.86-1.90 (m, 1H), 2.50-2.54 (dd, J=17 Hz, J=6 Hz, 1H), 2.59-2.64 (dd, J=16 Hz, J=7 Hz, 1H), 2.86-2.90 (t, J=7 Hz, 1H), 4.22 (s, 3H), 4.34-4.37 (m, 1H), 7.86 (s, 1H); ESIMS found for C₁₂H₁₇BN₄O₆S m/z 339.1 (M−H₂O)⁺.

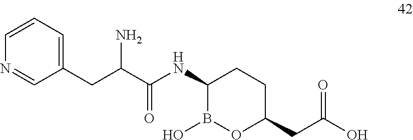 [SMILES: NC(Cc1cccnc1)C(=O)N[C@H]1CC[C@@H](CC(=O)O)OB1O]

2-((3R,6S)-3-(2-amino-3-(pyridin-3-yl)propanamido)-2-hydroxy-1,2-oxaborinan-6-yl)acetic acid 42 was isolated as the TFA salt. ¹H NMR (CD₃OD/CF₃O₂D) δ ppm 1.43-1.56 (m, 2H), 1.72-1.83 (m, 2H), 2.37-2.42 (m, 1H), 2.53-2.57 (t, J=6 Hz, 1H), 2.89-2.93 (t, J=7 Hz, 1H), 3.37-3.43 (m, 2H), 4.17-4.21 (t, J=7 Hz, 1H), 4.41-4.46 (m, 1H), 8.06-8.10 (dd, J=6 Hz, J=3 Hz, 1H), 8.53-8.57 (t, J=17 Hz, 1H), 8.80-8.81 (brd, J=4 Hz, 1H), 8.84-8.87 (brd, J=6 Hz, 1H); ESIMS found for C₁₄H₂₀BN₃O=m/z 304.2 (M−H₂O)⁺.

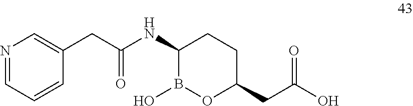 [SMILES: O=C(O)C[C@@H]1CC[C@H](NC(=O)Cc2cccnc2)B(O)O1]

2-((3R,6S)-2-hydroxy-3-(2-(pyridin-3-yl)acetamido)-1,2-oxaborinan-6-yl)acetic acid 43 was isolated as the TFA salt. ¹H NMR (CD₃OD) δ ppm 1.15-1.20 (m, 1H), 159-1.63 (m, 1H), 1.68-1.74 (m, 2H), 2.29-2.34 (dd, J=15 Hz, J=6 Hz, 2H), 2.66-2.68 (m, 1H), 3.94 (s, 2H), 4.11-4.18 (m, 1H), 7.82-7.85 (dd, J=8 Hz, J=6 Hz, 1H), 8.30-8.32 (d, J=8 Hz, 1H), 8.68-8.70 (brd, J=5 Hz, 1H), 8.72-8.75 (brs, 1H); ESIMS found for C₁₃H₁₇BN₂O5 m/z 275 (M−H₂O)⁺.

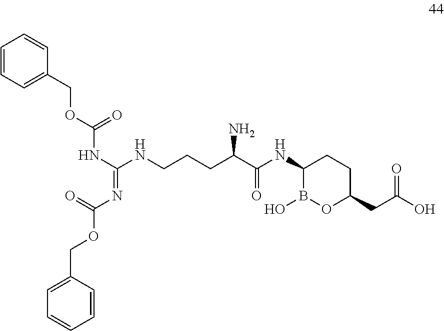 [SMILES: N[C@H](CCCN/C(=N/C(=O)OCc1ccccc1)NC(=O)OCc1ccccc1)C(=O)N[C@H]1CC[C@@H](CC(=O)O)OB1O]

2-((3R,6S)-3-((R)-2-amino-5-((Z)-2,3-bis(benzyloxycarbonyl)guanidino) pentanamido)-2-hydroxy-1,2-oxaborinan-6-yl)acetic acid 44 was isolated as the TFA salt. ¹H NMR (CD₃OD) δ ppm 1.28-1.34 (dd, J=18 Hz, J=12 Hz, 1H), 1.39-1.49 (m, 1H), 168-1.74 (m, 1H), 1.74-1.84 (m, 4H), 1.84-1.94 (m, 1H), 2.38-2.43 (dd, J=16 Hz, J=6 Hz 1H), 2.49-2.54 (dd, J=17 Hz, J=7 Hz, 1H), 2.72-2.75 (t, J=7 Hz, 1H), 3.90-3.99 (m, 3H), 4.28-4.31 (m, 1H), 5.11-5.17 (dd, J=16 Hz, J=13 Hz, 2H), 5.30 (s, 2H), 7.30-7.44 (m, 10H); ESIMS found for C₂₈H₃₆BN₅O₉ m/z 580 (M−H₂O)⁺.

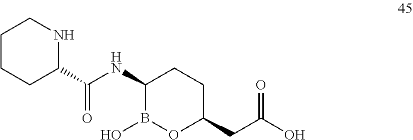

2-((3R,6S)-2-hydroxy-3-((S)-piperidine-2-carboxamido)-1,2-oxaborinan-6-yl)acetic acid 45 was isolated as the TFA salt. ¹H NMR (CD₃OD) δ ppm 1.44-1.51 (m, 1H), 154-1.80 (m, 5H), 1.80-1.91 (m, 2H), 1.91-1.98 (brd, J=12 Hz, 1H), 2.16-2.21 (dd, J=13 Hz, J=2 Hz, 1H), 2.49-2.57 (non, J=7 Hz, 2H), 2.75-2.78 (t, J=6 Hz, 1H), 2.98-3.03 (dt, J=13 Hz, J=3 Hz, 1H), 3.36-3.39 (d, J=13 Hz, 1H), 3.79-3.82 (dd, J=12 Hz, J=4 Hz, 1H), 4.34-4.38 (m, 1H); ESIMS found for C₁₂H₂O3N₂O₅ m/z 267 (M−H₂O)⁺.

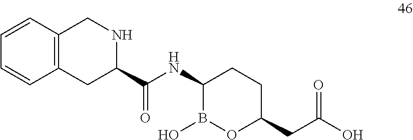 [SMILES: O=C(O)C[C@@H]1CC[C@H](NC(=O)[C@H]2Cc3ccccc3CN2)B(O)O1]

2-((3R,6S)-2-hydroxy-3-((R)-1,2,3,4-tetrahydroisoquinoline-3-carboxamido)-1,2-oxaborinan-6-yl)acetic acid 46 was isolated as the TFA salt. ¹H NMR (CD₃OD) δ ppm 1.43-1.51 (m, 1H), 1.56-163 (m, 1H), 1.75-183 (m, 1H), 1.86-1.94 (m, 1H), 2.46-2.57 (dq, J=16 Hz, J=6 Hz, 2H), 2.82-2.86 (t, J=7 Hz, 1H), 118-3.24 (dd, J=17 Hz, J=12 Hz, 1H), 3.36-3.41 (dd, J=17 Hz, J=5 Hz, 1H), 4.21-4.24 (dd, J=18 Hz, J=13 Hz, 1H), 4.36-4.40 (m, 1H), 4.42 (s, 2H), 7.23-7.25 (m, 1H), 7.27-7.33 (m, 3H); ESIMS found for C₁₆H₂₁BN₂O₅ m/z 315 (M−H₂O)⁺.

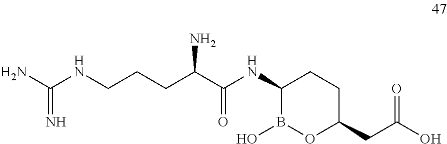 [SMILES: N=C(N)NCCC[C@@H](N)C(=O)N[C@H]1CC[C@@H](CC(=O)O)OB1O]

Following method E while the compound is still in 90% aq. trifluoroacetic acid (10 mL), 10% Pd/C (50 mg) was added. The reaction mixture was stirred under hydrogen for 6 h, filtered through Celite and rinsed with dichloroethane (10 mL). The filtrate was concentrated under vacuum and azeotroped with dichloroethane (2×10 mL). Triturating with ether resulted in a precipitate which was filtered and washed with ether (5 mL) and dried to give 2-((3R,6S)-34R)-2-amino-5-guanidinopentanamido)-2-hydroxy-1,2-oxaborinan-6-yl)acetic acid 47 as the TFA salt (50 mg) as an off-white solid. ¹H NMR (CD₃OD) δ ppm 1.39-1.46 (m, 1H), 1.52-1.58 (m, 1H), 1.66-1.77 (m, 2H), 1.77-1.84 (m, 1H), 1.87-1.95 (m, 3H), 2.34-2.38 (dd, J=17 Hz, J=3 Hz, 1H), 2.63-2.68 (dd, J=17 Hz, J=7 Hz, 1H), 2.94-2.97 (dd, J=10 Hz, J=6 Hz, 1H), 3.20-3.24 (dt, J=7 Hz, J=2 Hz, 2H), 3.86-3.88 (t, J=6 Hz, 1H), 4.27-4.31 (m, 1H); ESIMS found for C₁₂H₂₄BN₅O₅ m/z 312.2 (M−H₂O)⁺.

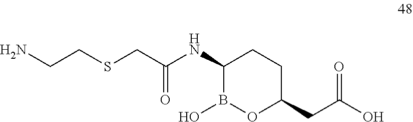 [SMILES: NCCSCC(=O)N[C@H]1CC[C@@H](CC(=O)O)OB1O]

2-((3R,6S)-3-(2-(2-aminoethylthio)acetamido)-2-hydroxy-1,2-oxaborinan-6-yl)acetic acid 48 was isolated as the TFA salt. ¹H NMR (CD₃OD) δ ppm 1.38-1.46 (m, 1H), 1.46-1.54 (m, 1H), 1.71-1.78 (m, 1H), 1.84-1.92 (m, 1H), 2.30-2.34 (dd, J=16 Hz, J=4 Hz, 1H), 2.56-2.61 (dd, J=16 Hz, J=6 Hz, 1H), 2.80-2.83 (t, J=6 Hz, 1H), 2.89-2.97 (non, J=7 Hz, 2H), 3.17-3.24 (non, J=5 Hz, 2H), 3.37 (s, 2H), 4.15-4.20 (m, 1H); ESIMS found for C₁₀H₁₉BN₂O₅S m/z 273 (M−H₂O)⁺.

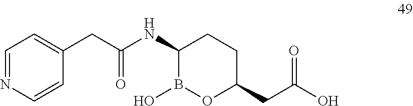 [SMILES: O=C(O)C[C@@H]1CC[C@H](NC(=O)Cc2ccncc2)B(O)O1]

2-((3R,6S)-2-hydroxy-3-(2-(pyridin-4-yl)acetamido)-1,2-oxaborinan-6-yl)acetic acid 49 was isolated as the TFA salt. ¹H NMR (CD₃OD) δ ppm 1.17-127 (m, 1H), 160-1.67 (m, 1H), 1.67-1.76 (m, 2H), 2.32-2.43 (m, 2H), 2.68-2.70 (t, J=4 Hz, 2H), 3.22-3.26 (t, J=7 Hz, 1H), 4.15-4.21 (m, 1H), 7.94-7.96 (d, J=7 Hz, 2H), 8.75-8.79 (d, J=6 Hz, 2H); ESIMS found for C₁₃H₁₇BN₂O₅ m/z 275.1 (M−H₂O)⁺.

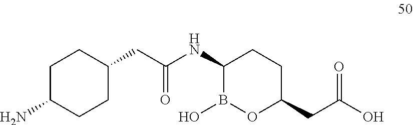 [SMILES: N[C@H]1CC[C@@H](CC(=O)N[C@H]2CC[C@@H](CC(=O)O)OB2O)CC1]

2-((3R,6S)-3-(2-(4-aminocyclohexyl)acetamido)-2-hydroxy-1,2-oxaborinan-6-yl)acetic acid 50 was isolated as the TFA salt. ¹H NMR (CD₃OD) δ ppm 1.15-1.25 (m, 1H), 1.44-1.88 (m, 10H), 2.05-2.13 (m, 1H), 2.19-2.21 (d, J=8 Hz, 1H), 2.30-2.36 (dd, J=6 Hz, 1H), 2.38-2.47 (m, 3H), 2.61-2.63 (brd, J=3 Hz, 1H), 3.18-3.22 (t, J=7 Hz, 1H), 4.04-4.11 (m, 1H); ESIMS found for C₁₄H₂₅BN₂O₅ m/z 295.1 (M−H₂O)⁺.

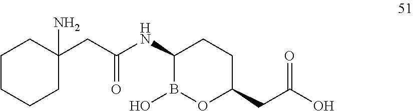 [SMILES: NC1(CC(=O)N[C@H]2CC[C@@H](CC(=O)O)OB2O)CCCCC1]

2-((3R,6S)-3-(2-(1-aminocyclohexyl)acetamido)-2-hydroxy-1,2-oxaborinan-6-yl)acetic acid 51 was isolated as the TFA salt. ¹H NMR (CD₃OD) δ ppm 1.23-1.34 (m, 1H), 134-1.48 (m, 1H), 1.48-1.86 (m, 12H), 2.40-2.50 (m, 2H), 2.65-2.83 (m, 2H), 3.22-3.26 (t, J=7 Hz, 1H), 4.11-4.18 (m, 1H); ESIMS found for C₁₄H₂₅BN₂O₅ m/z 295 (M−H₂O)⁺.

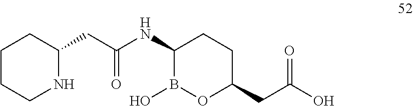 [SMILES: O=C(O)C[C@@H]1CC[C@H](NC(=O)C[C@H]2CCCCN2)B(O)O1]

2-((3R,6S)-2-hydroxy-3-(2-((R)-piperidin-2-yl)acetamido)-1,2-oxaborinan-6-yl)acetic acid 52 was isolated as the TFA salt. ¹H NMR (CD₃OD) δ ppm 1.27-137 (m, 1H), 1.49-1.80 (m, 7H), 1.86-2.00 (brdd, J=11 Hz, 3H), 2.44-2.46 (d, J=6 Hz, 2H), 2.61-2.65 (m, 1H), 2.72-2.73 (d, J=6 Hz, 1H), 3.03-3.09 (t, J=13 Hz, 1H), 3.41-3.45 (d, J=13 Hz, 1H), 3.47-3.56 (m, 1H), 4.15-4.21 (m, 1H); ESIMS found for C₁₃H₂₃BN₂O₅ m/z 281 (M−H₂O)⁺.

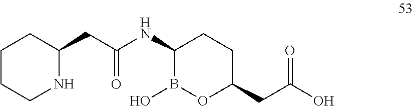 [SMILES: O=C(O)C[C@@H]1CC[C@H](NC(=O)C[C@@H]2CCCCN2)B(O)O1]

2-((3R,6S)-2-hydroxy-3-(2-((S)-piperidin-2-yl)acetamido)-1,2-oxaborinan-6-yl)acetic acid 53 was isolated as the TFA salt. ¹H NMR (CD₃OD) δ ppm 1.26-1.35 (m, 1H), 1.48-1.59 (m, 1H), 1.59-1.68 (m, 2H), 1.68-1.81 (m, 3H), 187-2.00 (m, 3H), 2.45-2.47 (d, J=7 Hz, 2H), 2.65-2.67 (t, J=4 Hz, 1H), 2.74-2.76 (t, J=6 Hz, 2H), 3.03-3.08 (dt, J=13 Hz, J=3 Hz, 1H), 3.42-3.46 (brd, J=13 Hz, 1H), 3.47-3.55 (m, 1H), 4.12-4.19 (m, 1H); ESIMS found for C₁₃H₂₃BN₂O₅ m/z 298.1 (M+H).

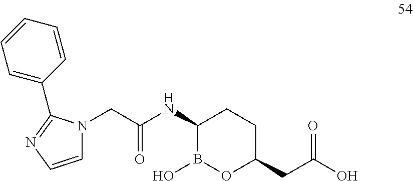 [SMILES: O=C(O)C[C@@H]1CC[C@H](NC(=O)Cn2ccnc2-c2ccccc2)B(O)O1]

2-((3R,6S)-2-hydroxy-3-(2-(2-phenyl-1H-imidazol-1-yl)acetamido)-1,2-oxaborinan-6-yl)acetic acid 54 was isolated as the TFA salt. ¹H NMR (CD₃OD) δ ppm 1.36-1.44 (m, 1H), 1.44-1.54 (m, 1H), 1.66-1.80 (m, 2H), 2.15 (s, 1H), 2.48-2.51 (m, J=6 Hz, 1H), 2.72-2.75 (t, J=7 Hz, 1H), 4.33-4.39 (m, 1H), 4.94-5.05 (m, 2H), 7.65-7.76 (m, 7H); ESIMS found for C₁₇H₂₀13 N₃O₅ m/z 358.2 (M+H).

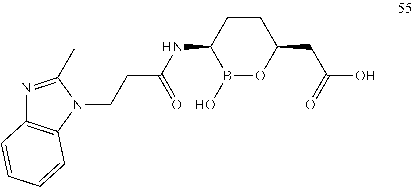 [SMILES: Cc1nc2ccccc2n1CCC(=O)N[C@H]1CC[C@@H](CC(=O)O)OB1O]

2-((3R,6S)-2-hydroxy-3-β-(2-methyl-1H-benzo[d]imidazol-1-yl)propanamido)-1,2-oxaborinan-6-yl)acetic acid 55. ¹H NMR (CD₃OD) δ ppm 0.92-1.00 (m, 1H), 1.47-1.53 (m, 1H), 1.58-1.62 (m, 2H), 2.31-2.33 (d, J=7 Hz, 2H), 2.50-2.52 (t, J=4 Hz, 1H), 2.97 (s, 3H), 3.08-3.20 (m, 2H), 4.04-4.10 (m, 1H), 4.77-4.81 (t, J=6 Hz, 2H), 7.61-7.68 (m, 2H), 7.75-7.78 (d, J=7 Hz, 1H), 7.93-7.95 (d, J=7 Hz, 1H); ESIMS found for C₁₇H₂₂BN₃O₅ m/z 342.2 (M−H₂O)⁺.

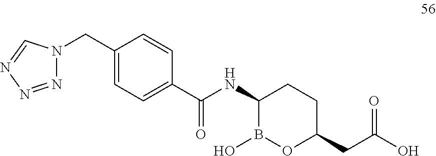 [SMILES: O=C(O)C[C@@H]1CC[C@H](NC(=O)c2ccc(Cn3cnnn3)cc2)B(O)O1]

2-((3R,6S)-3-(4-((1H-tetrazol-1-yl)methyl)benzamido)-2-hydroxy-1,2-oxaborinan-6-yl)acetic acid 56. ¹H NMR (CD₃OD) δ ppm 1.10-1.21 (m, 1H), 158-1.64 (m, 1H), 170-1.79 (m, 1H), 1.89-1.96 (m, 1H), 2.31-2.36 (dd, J=15 Hz, J=6 Hz, 1H), 2.41-2.47 (m, 1H), 2.80-2.83 (brd, J=4 Hz, 1H), 4.11-4.17 (m, 1H), 5.83 (s, 2H), 7.53-7.55 (d, J=8 Hz, 2H), 8.02-8.05 (d, J=8 Hz, 2H), 9.30 (s, 1H); ESIMS found for C₁₅H₁₈BN₅O₅ m/z 342.0 (M−H₂O)⁺.

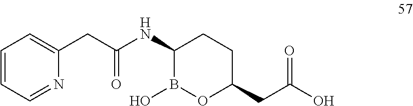 [SMILES: O=C(O)C[C@@H]1CC[C@H](NC(=O)Cc2ccccn2)B(O)O1]

2-((3R,6S)-2-hydroxy-3-(2-(pyridin-2-yl)acetamido)-1,2-oxaborinan-6-yl)acetic acid 57 was isolated as the TFA salt. ¹H NMR (CD₃OD) δ ppm 1.21-132 (m, 1H), 159-1.67 (m, 2H), 167-1.75 (m, 2H), 2.29-2.40 (m, 3H), 2.67-2.72 (m, 1H), 4.14-4.21 (m, 1H), 7.62-7.66 (t, J=6 Hz, 1H), 7.70-7.73 (d, J=8 Hz, 1H), 8.14-8.18 (t, J=8 Hz, 1H), 8.65-8.67 (d, J=5 Hz, 1H); ESIMS found for C₁₃H₁₇BN₂O₅ m/z 275.1 (M−H₂O)⁺.

The following compounds are prepared in accordance with the procedure described in the above Example 1 using methods C and E.

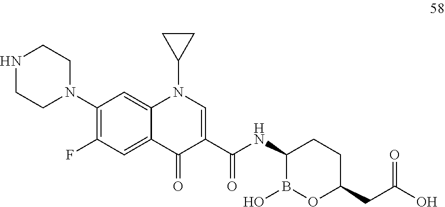 [SMILES: O=C(O)C[C@@H]1CC[C@H](NC(=O)c2cn(C3CC3)c3cc(N4CCNCC4)c(F)cc3c2=O)B(O)O1]

2-((3R,6S)-3-(1-cyclopropyl-6-fluoro-4-oxo-7-(piperazin-1-yl)-1,4-dihydroquinoline-3-carboxamido)-2-hydroxy-1,2-oxaborinan-6-yl)acetic acid 58 was isolated as the TFA salt. ¹H NMR (CD₃OD) δ ppm 1.14-1.29 (m, 3H), 1.39-1.44 (brd, J=7 Hz, 2H), 1.56-1.63 (dd, J=14 Hz, J=3 Hz, 1H), 1.70-1.80 (m, 1H), 1.92-1.99 (d, J=14 Hz, 1H), 2.33-2.38 (dd, J=15 Hz, J=6 Hz, 1H), 2.43-2.48 (dd, J=15 Hz, J=7 Hz, 1H), 2.85-2.86 (d, J=3 Hz, 1H), 3.46-3.52 (m, 4H), 3.59-3.64 (m, 4H), 3.73-3.79 (m, 1H), 4.08-4.15 (m, 1H), 7.66-7.67 (d, J=7 Hz, 1H), 8.00-8.03 (d, J=13 Hz, 1H), 8.81 (s, 1H); ESIMS found for C₂₃H₂₈BFN₄O₆ m/z 469.2 (M−H₂O)⁺.

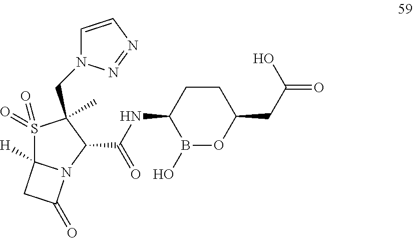 [SMILES: C[C@]1(Cn2ccnn2)[C@H](C(=O)N[C@H]2CC[C@@H](CC(=O)O)OB2O)N2C(=O)C[C@H]2S1(=O)=O]

2-[(3R,6S)-2-hydroxy-3-[(2S,3S,5R)-3-methyl-4,4,7-trioxo-3-(1H-1,2,3-triazol-1-ylmethyl)-4λ⁶-thia-1-azabicyclo[3.2.0]heptane-2-amido]-1,2-oxaborinan-6-yl]acetic acid 59. ¹H NMR (CD₃OD) δ ppm 1.43 (s, 3H), 1.49-157 (m, 1H), 172-1.81 (m, 3H), 2.51-2.56 dd, J=15 Hz, J=6 Hz, 1H), 2.62-2.67 (dd, J=15 Hz, J=8 Hz, 1H), 2.80-2.84 (m, 1H), 3.41-3.44 (dd, J=17 Hz, J=2 Hz, 1H), 3.63-3.67 (dd, J=16 Hz, J=5 Hz, 1H), 4.37-4.44 (m, 1H), 4.61 (s, 1H), 4.90-4.94 (dd, J=5 Hz, J=2 Hz, 1H), 5.16-5.19 (d, J=15 Hz, 1H), 5.25-5.28 (d, J=15 Hz, 1H), 7.77 (s, 1H), 8.07 (s, 1H); ESIMS found for C₁₆H₂₂BN₅O₈S m/z 438 (M−H₂O)⁺.

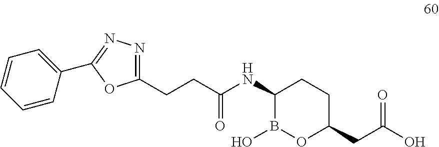 [SMILES: O=C(O)C[C@@H]1CC[C@H](NC(=O)CCc2nnc(-c3ccccc3)o2)B(O)O1]

2-((3R,6S)-2-hydroxy-3-(3-(5-phenyl-1,3,4-oxadiazol-2-yl)propanamido)-1,2-oxaborinan-6-yl)acetic acid 60. ¹H NMR (CD₃OD) δ ppm 1.10-1.21 (m, 1H), 1.50-1.58 (dd, J=14 Hz, J=3 Hz, 1H), 1.59-1.68 (dt, J=11 Hz, J=5 Hz, 1H), 1.74-1.81 (brd, J=13 Hz, 1H), 2.22-2.26 (dd, J=15 Hz, J=6 Hz, 1H), 2.30-2.34 (dd, J=15 Hz, J=7 Hz, 1H), 2.63-2.64 (d, J=4 Hz, 1H), 3.01-3.12 (sex, J=7 Hz, 2H), 3.33-3.43 (sex, J=7 Hz, 2H), 4.03-4.09 (m, 1H), 7.54-7.62 (m, 3H), 8.03-8.05 (d, J=8 Hz, 2H); ESIMS found for C₁₇H₂₀BN₃O₆ m/z 356.1 (M−H₂O)⁺.

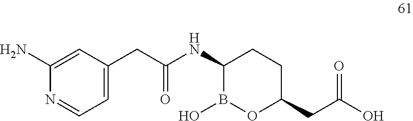 [SMILES: Nc1cc(CC(=O)N[C@H]2CC[C@@H](CC(=O)O)OB2O)ccn1]

2-((3R,6S)-3-(2-(2-aminopyridin-4-yl)acetamido)-2-hydroxy-1,2-oxaborinan-6-yl)acetic acid 61 was isolated as the TFA salt. ¹H NMR (CD₃OD) δ ppm 1.58-1.66 (m, 1H), 167-1.78 (m, 3H), 2.31-2.36 (dd, J=15 Hz, J=6 Hz, 1H), 2.39-2.44 (dd, J=15 Hz, J=7 Hz, 1H), 2.65-2.68 (t, J=4 Hz, 1H), 4.12-4.19 (m, 1H), 6.85-6.87 (d, J=7 Hz, 1H), 6.99 (s, 1H), 7.81-7.82 (d, J=7 Hz, 1H); ESIMS found for C₁₃H₁₈BN₃O₅ m/z 290.1 (M−H₂O)⁺.

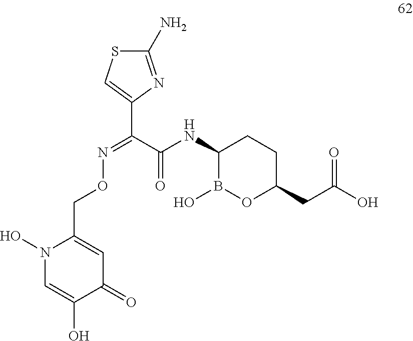 [SMILES: Nc1nc(/C(=N/OCc2cc(=O)c(O)cn2O)C(=O)N[C@H]2CC[C@@H](CC(=O)O)OB2O)cs1]

Following method E, the reaction mixture was evaporated in vacuo, azeotroped with MeCN (3×5 mL) and the residue was triturated with ether (5 mL). The precipitate was filtered, dissolved in dioxane-water mixture and freeze dried to get 2-((3R)-3-((Z)-2-(2-aminothiazol-4-yl)-2-((1,5-dihydroxy-4-oxo-1,4-dihydropyridin-2-yl) methoxyimino)acetamido)-2-hydroxy-1,2-oxaborinan-6-yl)acetic acid 62 as the TFA (25 mg) salt as a fluffy solid. ESIMS found for C₁₇H₂₀13 N₅O₉S m/z 464.0 (M−H₂O)⁺.

Synthesis of 2-((3R)-3-amino-2-hydroxy-1,2-oxaborinan-6-yl)acetic acid hydrochloride 7. An example synthesis of 7 is depicted in Scheme 10 and Example 2.

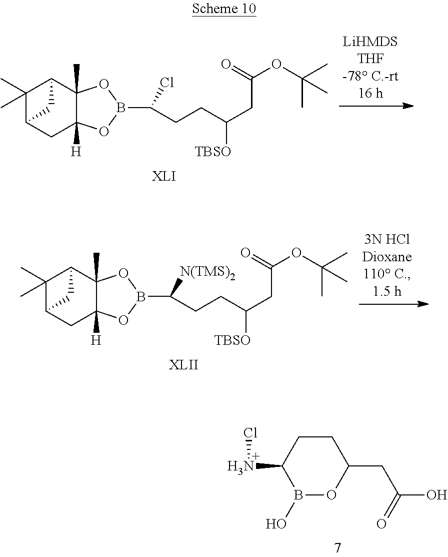

### Example 2

### Step 1

6-(tert-butoxy)-4-[(tert-butyldimethylsilyl)oxy]-1-chloro-6-oxo-1-[(2S,6R)-2,9,9-trimethyl-3,5-dioxa-4-boratricyclo[6.1.1.02,6]decan-4-yl]hexane XLI (515 mg, 0.97 mmol) in THF (5 mL) was cooled to −78° C. under nitrogen. A solution of LiHMDS (1 mL, 1.0 M in THF, 1 mmol, 1.0 eq) was added slowly and the reaction flask was then allowed to warm to room temperature where it was stirred for 16 h. The yellow solution was concentrated under reduced pressure to give an oil. After hexane (10 mL) was added to the oil, a precipitate formed. This was then filtered through Celite and the filtrate concentrated under reduced pressure to give 1-[bis(trimethylsilyl)amino]-6-(tert-butoxy)-4-[(tert-butyldimethylsilyl)oxy]-6-oxo-1-[(2S,6R)-2,9,9-trimethyl-3,5-di oxa-4-boratricyclo[6.1.1.02,6]decan-4-yl]hexyl XLII.

### Step 2

The procedure is identical to that found in Example 1 method D. Compound 7 was isolated as a white powder (120 mg, 0.573 mmol, 59.1% yield). ¹H NMR (CD₃OD) δ ppm 1.43-1.66 (m, 1H), 1.66-1.79 (m, 1H), 1.79-1.97 (m, 1H), 1.97-2.30 (m, 1H), 2.40-2.71 (m, 3H), 4.34-4.54 (m, 1H); ESIMS found for C₆H₁₂BNO₄ m/z 174 (63%) (M+H).

Synthesis of 2-((3R)-2-hydroxy-3-(2-(thiophen-2-yl)acetamido)-1,2-oxaborepan-7-yl)acetic acid 63. An example synthesis of 63 is depicted in Scheme 11 and

### Example 3

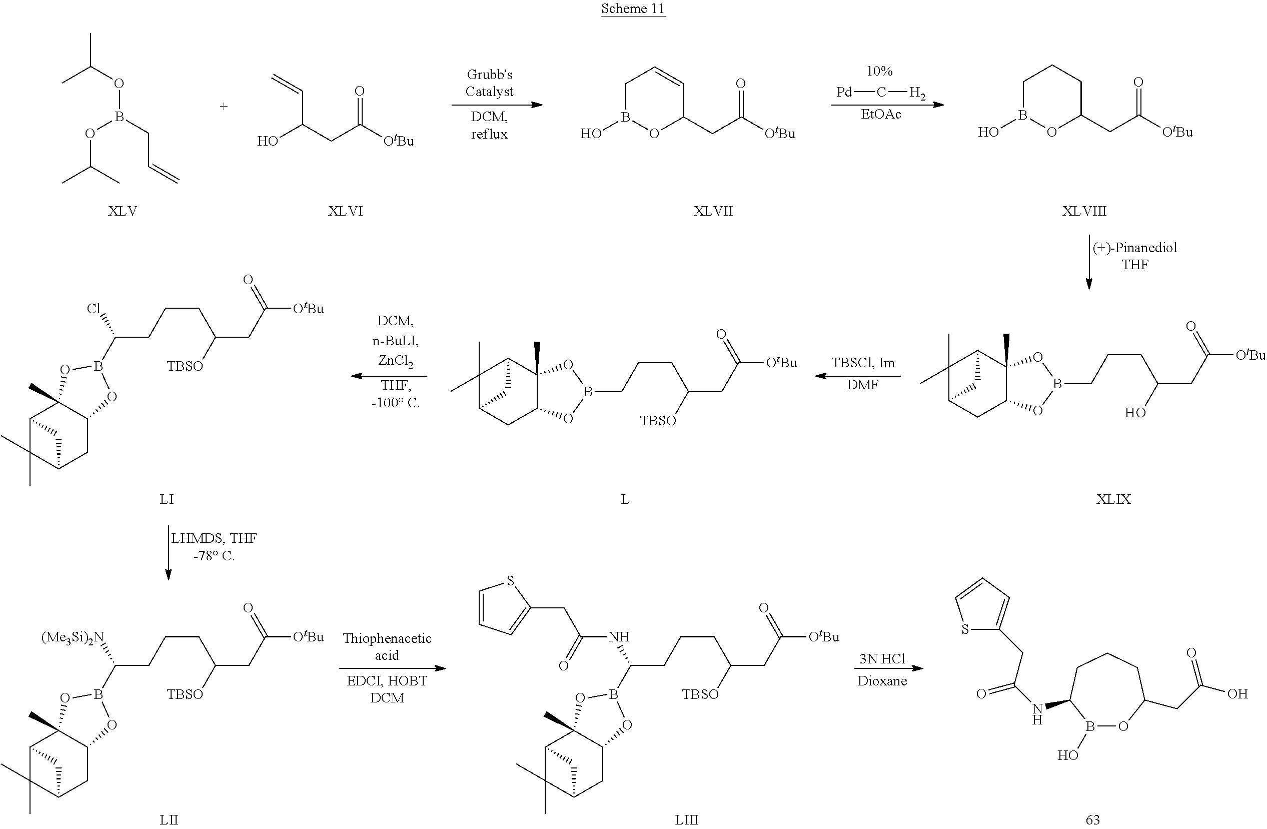

### Example 3

### Step 1

To a solution of tert-butyl 3-hydroxypent-4-enoate, XLVI (674 mg, 3.92 mmol) in DCM (15 mL) was added diisopropylallylboronate XLV(2 g, 11.76 mmol) via syringe. To the mixture was then added Grubbs' first generation catalyst (260 mg, 0.31 mmol, 7.5 mol %) and the vessel was purged with argon. The reaction was heated at 65° C. under nitrogen for 18 h. The mixture was concentrated under vacuum and the residue was purified by flash column chromatography (100% hexane→30% EtOAc/hexane) to afford tert-butyl 2-(2-hydroxy-3,6-dihydro-2H-1,2-oxaborinin-6-yl)acetate XLVII (770 mg, 3.63 mmol, 92.7% yield).

### Step 2

To a solution of tert-butyl 2-(2-hydroxy-3,6-dihydro-2H-1,2-oxaborinin-6-yl)acetate XLVII (670 mg, 3.16 mmol) in EtOAc (45 mL) was added 10% Pd/C (135 mg). The vessel was evacuated by applying vacuum and flushed with hydrogen gas. The reaction was stirred under hydrogen for 2 h. The mixture was filtered through a Celite pad and which was washed with additional EtOAc (15 mL). Concentration of the filtrate gave pure tert-butyl 2-(2-hydroxy-1,2-oxaborinan-6-yl)acetate XLVIII (641 mg, 3.00 mmol, 94.8% yield).

### Step 3

To a solution of tert-butyl 2-(2-hydroxy-1,2-oxaborinan-6-yl)acetate XLVIII (641 mg, 3.00 mmol) in THF (20 mL) was added (1 S,2S,3R,5S)-2,6,6-trimethylbicyclo[3.1.1]heptane-2,3-diol (509 mg, 3 mol) at room temperature. The reaction mixture was stirred for 16 h and concentrated under vacuum. The residue was purified by column chromatography (100% hexane→40% EtOAc/hexane) on silica gel to give tert-butyl 3-hydroxy-6-[(1R,2R,6S,8R)-6,9,9-trimethyl-3,5-dioxa-4-boratricyclo[6.1.1.0^{2,6}]decan-4-yl]hexanoate XLIX (790 mg, 2.16 mmol, 71.9% yield).

### Step 4

To a solution of alcohol XLIX (790 mg, 2.16 mmol) in DMF (7.5 mL) was added imidazole (548 mg, 8.06 mmol) followed by TBDMSCl (580 mg, 3.87 mol). The reaction mixture was stirred at room temperature for 16 h and concentrated under vacuum. The white slurry was dissolved in 100 mL of EtOAc and washed with saturated NaHCO₃ solution (20 mL), water (2×10 mL) and dried (Na₂SO₄). The organic extract was concentrated under vacuum and the residue was purified by column chromatography (100% hexane→30% EtOAc/hexane) on silica gel to give tert-butyl 3-[(tert-butyldimethylsilyl)oxy]-6-[(1R,2R,6S,8R)-6,9,9-trimethyl-3,5-dioxa-4-boratricyclo[6.1.1.0^{2,6}]decan-4-yl]hexanoate L (1 g, 2.08 mmol, 96.3% yield).

### Step 5

To a solution of DCM (0.26 mL, 4.16 mmol) in THF (5 mL) at −100° C. was added 2.5 M n-butyl lithium in hexane (1 mL, 2.5 mmol) slowly under nitrogen and down the inside wall of the flask whilst maintaining the temperature below −90° C. The resulting white precipitate was stirred for 30 minutes before the addition of L (1 g, 2.08 mmol) in THF (3 mL) at −90° C. Zinc chloride (5 mL, 0.5 M in THF, 2.5 mmol) was then added to the reaction mixture at −90° C. and then the reaction was allowed to warm to room temperature where it was stirred for 16 h. The reaction was quenched with a saturated solution of ammonium chloride and the phases were separated. The aqueous phase was then extracted with diethyl ether (2×10 mL) and the combined organic extracts were dried over Na₂SO₄, filtered and concentrated under reduced pressure. The concentrated material was then chromatographed (100% hexane→20% EtOAc-hexane) to obtain tert-butyl(7S)-3-[(tert-butyldimethylsilyl)oxy]-7-chloro-7-[(1R,2R,6S,8R)-6,9,9-trimethyl-3,5-dioxa-4-boratricyclo[6.1.1.0^{2,6}]decan-4-yl]heptanoate LI (740 mg, 1.40 mmol, 67.2% yield).

### Step 6

Chloro intermediate LI (727 mg, 1.37 mmol) in THF (7 mL) was cooled to −78° C. under nitrogen. A solution of 1M LiHMDS solution in THF (1.37 mL, 1.37 mmol) was added slowly at −78° C. Upon completion of the addition, the reaction flask was allowed to warm to room temperature. After stirring at room temperature for 16 h, the reaction mixture was concentrated under vacuum and hexane (20 mL) was added The precipitated lithium salts were filtered off through a Celite pad, rinsed with additional hexane and the combined filtrates were concentrated under vacuum to give crude tert-butyl(7S)-7-[bis(trimethylsilyl)amino]-3-[(tert-butyldimethylsilyl)oxy]-7-[(1R,2R,6S,8R)-6,9,9-trimethyl-3,5-dioxa-4-boratricyclo[6.1.1.0^{2,6}]decan-4-yl]heptanoate LII.

### Step 7

To a stirred solution of 2-thiophenacetic acid (232 mg, 1.64 mmol) in DCM (45 mL) at 0° C. under nitrogen was added EDCI (391 mg, 2.05 mmol) and HOBT (221 mg, 1.64 mmol). After stirring at 0° C. for 30 minutes, a solution of the bis-silyl amide LII intermediate (1.37 mmol) in DCM (10 mL) followed by N-methyl-morpholine (0.3 mL, 2.74 mmol) were sequentially added at 0° C. Upon completion of the addition, the reaction flask was allowed to warm to room temperature. After stirring at room temperature overnight, the reaction mixture was washed with water, dried and concentrated under vacuum. The residue was purified by column chromatography (100% DCM→50% EtOAc/DCM) to afford tert-butyl(7S)-3-[(tert-butyldimethylsilyl)oxyl-7-[2-(thiophen-2-yl)acetamido]-7-1(1R,2R,6S,8R)-6,9,9-trimethyl-3,5-dioxa-4-boratricyclo[6.1.1.0^{2,6}]decan-4-yl]heptanoate LIII (340 mg, 0.54 mmol, 39.4% yield for 2 steps).

### Step 8

To a solution of amide LIII (300 mg, 0.47 mmol) in 1,4-dioxane (9 mL) was added 9 mL of 3 N HCl. The reaction mixture was heated at reflux for 90 minutes. The cooled reaction mixture was then diluted with water (10 mL) and extracted with diethyl ether (2×10 mL). The aqueous layer was concentrated to afford a sticky solid which was azeotroped with MeCN (3×10 mL). The residue was dissolved in 40% dioxane-water and lyophilized to afford 2-((3R)-2-hydroxy-3-(2-(thiophen-2-yl)acetamido)-1,2-oxaborepan-7-yl)acetic acid 63 as an off-white solid (100 mg, 32.1 mmol, 68.4% yield). ¹H NMR (CD₃OD) δ ppm 1.21-1.38 (m, 2H), 1.42-1.60 (m, 2H), 1.60-1.72 (m, 1H), 1.80-1.94 (m, 1H), 2.32-2.47 (m, 2H), 2.54-2.58 (dd, J=15 Hz, J=6 Hz, 1H), 3.97-3.98 (d, J=8 Hz, 1H), 4.05 (s, 2H), 6.97-7.01 (m, 1H), 7.02-7.10 (m, 1H), 7.33-7.37 (m, 1H); ESIMS found for C₁₃H₁₈BNO₅S m/z 294.0 (M−H₂O)⁺.

Synthesis of 2-((3R)-2-hydroxy-3-(2-(thiophen-2-yl)acetamido)-2,3,4,7-tetrahydro-1,2-oxaborepin-7-yl)acetic acid 64. An example synthesis of 64 is depicted in Scheme 12 and Example 4.

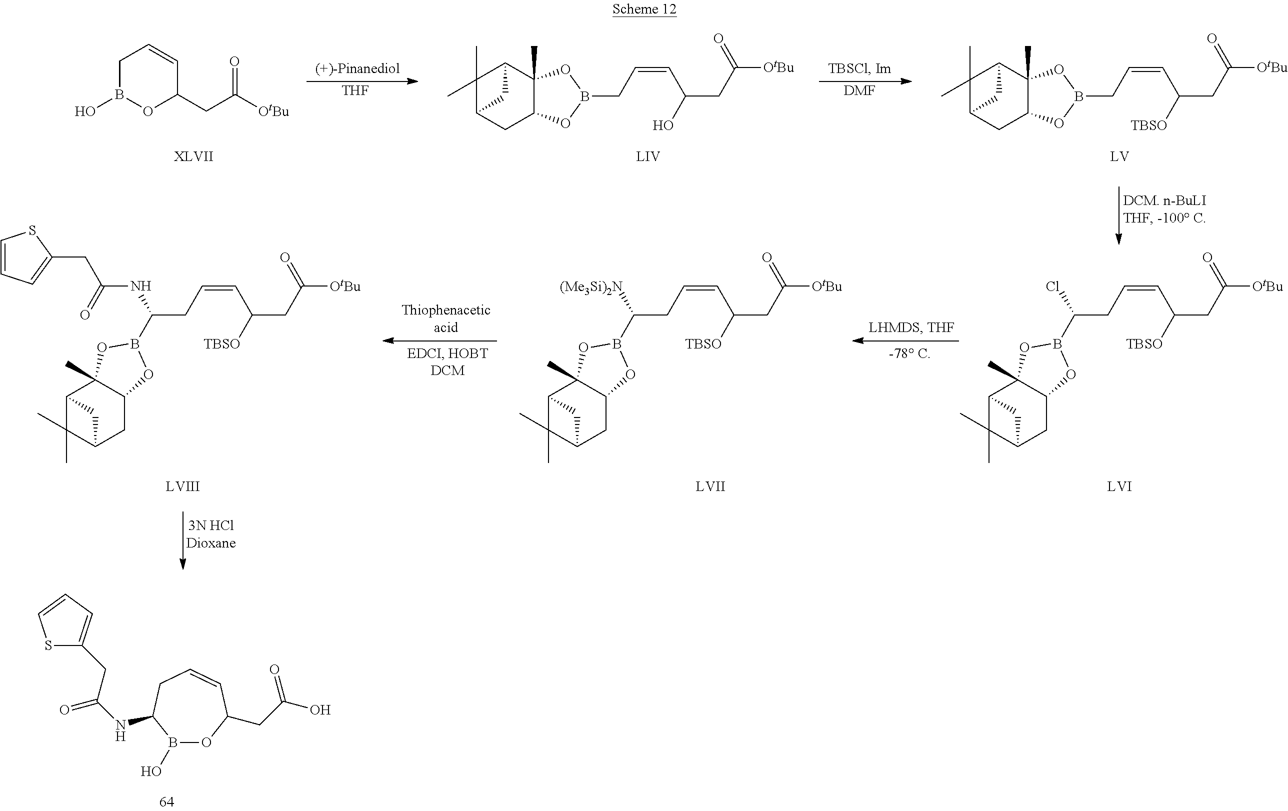

### Example 4

### Step 1

To a stirred solution of tert-butyl 2-(2-hydroxy-3,6-dihydro-2H-1,2-oxaborinin-6-yl)acetate XLVII (770 mg, 4.58 mmol) in THF (25 mL) was added (1 S,2S,3R,5S)-2,6,6-trimethylbicyclo[3.1.1]heptane-2,3-diol (980 mg, 4.58 mmol) at room temperature. The reaction mixture was stirred for 16 h and concentrated under vacuum. The residue was purified by column chromatography (100% hexane→30% EtOAc/hexane) on silica gel to give tert-butyl(4Z)-3-hydroxy-6-[(1R,2R,6S,8R)-6,9,9-trimethyl-3,5-dioxa-4-boratricyclo[6.1.1.02,6]decan-4-yl]hex-4-enoate LIV (1 g, 2.75 mmol, 59.9% yield).

### Step 2

To a solution of alcohol LIV (650 mg, 1.78 mmol) in DMF (10 mL) was added imidazole (484 mg, 7.12 mmol) followed by TBDMSCl (534 mg, 3.56 mol). The reaction mixture was stirred at room temperature for 16 h and concentrated under vacuum. The white slurry was dissolved in 100 mL of EtOAc and washed with water (2×10 mL), brine and dried (Na2SO4). The organic extract was concentrated under vacuum and the residue was purified by column chromatography (100% hexane→20% EtOAc/hexane) on silica gel to give tert-butyl (4Z)-3-[(tert-butyldimethylsilyl)oxy]-6-[(1R,2R,6S,8R)-6,9,9-trimethyl-3,5-dioxa-4-boratricyclo[6.1.1.02,6]decan-4-yl]hex-4-enoate LV (800 mg, 1.67 mmol, 93.9% yield).

### Step 3

To a solution of DCM (0.3 mL, 4.68 mmol) in THF (8 mL) at −100° C. was added 2.5 M n-butyl lithium in hexane (1.12 mL, 2.8 mmol) slowly under nitrogen and down the inside wall of the flask whilst maintaining the temperature below −90° C. The resulting white precipitate was stirred for 30 minutes before the addition of LV (1.12 g, 2.34 mmol) in THF (3 mL) at −90° C. and the reaction was allowed to warm to room temperature where it was stirred for 16 h. The reaction was quenched with a saturated solution of ammonium chloride and the phases were separated. The aqueous phase was then extracted with diethyl ether (2×10 mL) and the combined organic extracts were dried over Na₂SO₄, filtered and concentrated under reduced pressure. The concentrated material was then chromatographed (100% hexane→20% EtOAc/hexane) to obtain tert-butyl(4Z,7S)-3-[(tert-butyldimethylsilyl)oxy]-7-chloro-7-[(1R,2R,6S,8R)-6,9,9-trimethyl-3,5-dioxa-4-boratricyclo[6.1.1.0^{2,6}]decan-4-yl]hept-4-enoate LVI (820 mg, 1.56 mmol, 66.5% yield).

### Step 4

Chloro intermediate LVI (790 mg, 1.49 mmol) in THF (10 mL) was cooled to −78° C. under nitrogen. A solution of 1M LiHMDS solution in THF (1.5 mL, 1.5 mmol) was added slowly at −78° C. Upon completion of the addition, the reaction flask was allowed to warm to room temperature. After stirring at room temperature for 16 h, the reaction mixture was concentrated under vacuum and hexane (20 mL) was added. The precipitated lithium salts were filtered off through a Celite pad, rinsed with additional hexane and the combined filtrates were concentrated under vacuum to give crude tert-butyl(4Z,7S)-7-[bis(trimethylsilyl)amino]-3-[(tert-butyldimethylsilyl)oxy]-7-[(1R,2R,6S,8R)-6,9,9-trimethyl-3,5-dioxa-4-boratricyclo[6.1.1.0^{2,6}]decan-4-yl]hept-4-enoate LVII.

### Step 5

To a stirred solution of 2-thiophenacetic acid (252 mg, 1.78 mmol) in DCM (35 mL) at 0° C. under nitrogen was added EDCI (426 mg, 2.23 mmol) and HOBT (240 mg, 1.78 mmol). After stirring at 0° C. for 30 minutes, a solution of the crude bis-silyl amide LVII intermediate in DCM (10 mL) followed by N-methyl-morpholine (0.32 mL, 3 mmol) were sequentially added at 0° C. Upon completion of the addition, the reaction flask was allowed to warm to room temperature. After stirring at room temperature overnight, the reaction mixture was washed with water, dried and concentrated under vacuum. The residue was purified by column chromatography (100% DCM→25% EtOAc/DCM) to afford tert-butyl(4Z,7S)-3-[(tert-butyldimethylsilyl)oxy]-7-[2-(thiophen-2-yl)acetamido]-7-[(1R,2R,6S,8R)-6,9,9-trimethyl-3,5-dioxa-4-boratricyclo[6.1.1.02,6]decan-4-yl]hept-4-enoate LVIII (600 mg, 0.95 mmol, 63.7% yield for 2 steps).

### Step 6

A solution of amide LVIII (100 mg, 0.15 mmol) in anisole (5 mL) at 0° C. was treated with pre-cooled 90% aq trifluoroacetic acid (10 mL). The reaction mixture was warmed to room temperature and stirred for 16 h. The mixture was evaporated in vacuo, azeotroped with MeCN (3×5 mL). The residue was sonicated in water (10 mL) and ether (10 mL). The aqueous phase was separated, washed with ether (2×5 mL) and freeze dried to give fluffy solid 2-((3R)-2-hydroxy-3-(2-(thiophen-2-yl)acetamido)-2,3,4,7-tetrahydro-1,2-oxaborepin-7-yl)acetic acid 64 (15 mg, 0.05 mmol, 32.3% yield). ¹H NMR (CD₃OD) δ ppm 2.23-2.35 (m, 2H), 2.40-2.61 (m, 2H), 2.76-2.83 (m, 1H), 3.96-4.03 (m, 1H), 4.10 (s, 2H), 5.34-5.40 (m, 1H), 5.53-5.74 (m, 1H), 6.97-7.08 (m, 2H), 7.32-7.39 (m, 1H); ESIMS found for C₁₃H₁₆BNO₅S m/z 292 (M−H₂O)⁺.

Synthesis of ethyl 2-((3R,6S)-2-hydroxy-3-(2-(thiophen-2-yl)acetamido)-1,2-oxaborinan-6-yl)acetate 65. An example synthesis of 65 is depicted in Scheme 13 and Example 5.

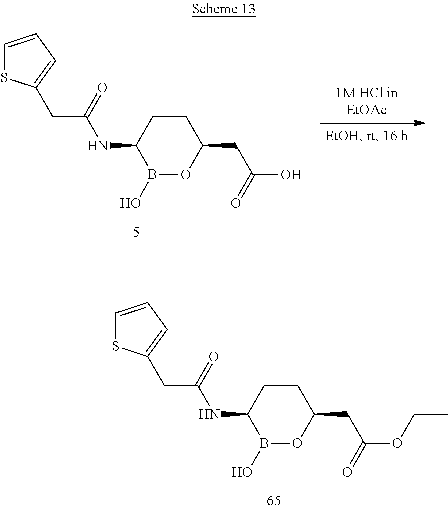 [SMILES: O=C(O)C[C@@H]1CC[C@H](NC(=O)Cc2cccs2)B(O)O1 ; CCOC(=O)C[C@@H]1CC[C@H](NC(=O)Cc2cccs2)B(O)O1]

### Example 5

### Step 1

To a solution of 5 (400 mg, 1.35 mmol) in 4 mL of absolute ethanol was added anhydrous 1M HCl in EtOAc (4 mL, 4 mmol). The reaction was stirred at room temperature for 16 h. The mixture was then concentrated and azeotroped with acetonitrile (3×10 mL) to give a sticky solid. Ether (10 mL) was added to the azeotroped sticky solid and the resulting precipitate was filtered. The filtered solid was rinsed with additional ether (5 mL) and dried to give ethyl 2-((3R,6S)-2-hydroxy-3-(2-(thiophen-2-yl)acetamido)-1,2-oxaborinan-6-yl)acetate 65 (300 mg, 0.92 mmol, 68.5% yield). 1H NMR (CD3OD) δ ppm 0.98-1.09 (q, J=14 Hz, 1H), 1.23-1.26 (t, J=7 Hz, 3H), 1.49-1.54 (dd, J=14 Hz, J=3 Hz, 1H), 157-1.64 (dt, J=11 Hz, J=2 Hz, 1H), 1.72-1.78 (brd, J=14 Hz, 1H), 2.24-2.28 (dd, J=15 Hz, J=6 Hz, 1H), 2.34-2.39 (dd, J=15 Hz, J=8 Hz, 1H), 2.63 (brs, 1H), 3.99 (s, 2H), 4.07-4.13 (q, J 4 Hz, 3H), 6.99-7.01 (t, J=4 Hz, 1H), 7.05-7.06 (d, J=3 Hz, 1H), 7.35-7.36 (dd, J=5 Hz, J=1.3 Hz, 1H); ESIMS found for C14H20BNO5S m/z 308.1 (M−H20)+.

Synthesis of 2-((3R,7R)-2-hydroxy-3-(2-(thiophen-2-yl)acetamido)-2,3,4,7-tetrahydro-1,2-oxaborepin-7-yl)acetic acid 67. An example synthesis of 67 is depicted in Scheme 14 and Example 6.

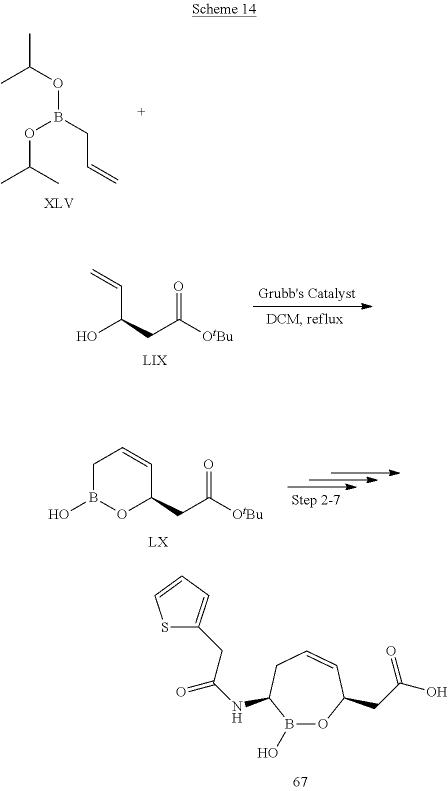 [SMILES: C=CCB(OC(C)C)OC(C)C ; C=C[C@H](O)CC(=O)OC(C)(C)C ; CC(C)(C)OC(=O)C[C@@H]1C=CCB(O)O1 ; O=C(O)C[C@@H]1C=CC[C@H](NC(=O)Cc2cccs2)B(O)O1]

### Example 6

### Step 1

Prepared starting from enantiomerically pure (R)-tert-butyl 3-hydroxypent-4-enoate [J. Am. Chem. Soc. (2007), 129, 4175-4177] in accordance with the procedure described in the above Step 1 of Example 3

### Steps 2-7

Prepared in accordance with the procedure described in the above Steps 1-6 of Example 4.

White fluffy solid (23 mg, 0.074 mmol, 47% yield). ¹H NMR (CD₃OD) δ ppm 2.29-2.31 (m, 1H), 2.40-2.68 (m, 4H), 4.10 (m, 2H), 4.74-4.82 (m, 1H), 5.35-5.38 (m, 1H), 5.53-5.58 (m, 1H), 6.98-7.05 (m, 2H), 7.32-7.36 (m, 1H); ESIMS found for C₁₃H₁₆BNO₅S m/z 292 (M−H₂O)⁺.

Synthesis of 2-((3R,7S)-2-hydroxy-3-(2-(thiophen-2-yl)acetamido)-2,3,4,7-tetrahydro-1,2-oxaborepin-7-yl)acetic acid 68. An example synthesis of 68 is depicted in Scheme 15 and Example 7.

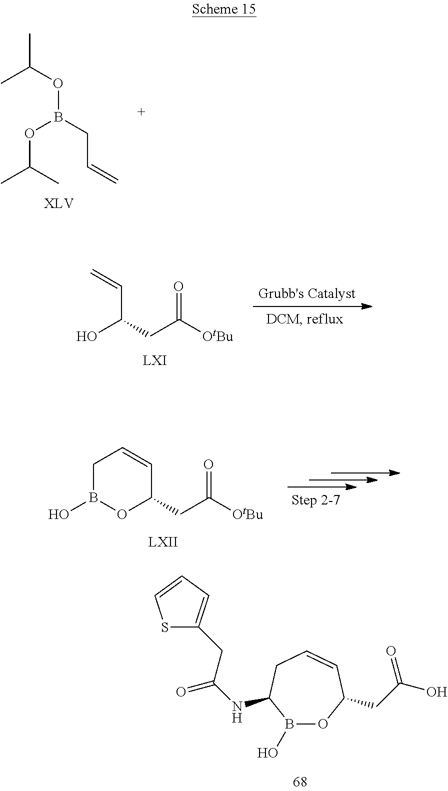 [SMILES: C=CCB(OC(C)C)OC(C)C ; C=C[C@@H](O)CC(=O)OC(C)(C)C ; CC(C)(C)OC(=O)C[C@H]1C=CCB(O)O1 ; O=C(O)C[C@H]1C=CC[C@H](NC(=O)Cc2cccs2)B(O)O1]

### Example 7

### Step 1

Prepared starting from enantiomerically pure (S)-tert-butyl 3-hydroxypent-4-enoate [J. Med. Chem., (2010), 53, 4654-4667] in accordance with the procedure described in the above Step 1 of Example 3

### Steps 2-7

Prepared in accordance with the procedure described in the above Steps 1-6 of Example 4.

White fluffy solid (45 mg, 0.146 mmol, 39% yield). ¹H NMR (CD₃OD) 6 ppm 2.15-2.18 (m, 1H), 2.29-2.38 (m, 2H), 2.66-2.72 (m, 2H), 3.88-3.91 (m, 1H) 4.00 (s, 2H), 5.24-5.27 (m, 1H), 5.57-5.63 (m, 1H), 6.87-6.96 (m, 2H), 7.24-7.28 (m, 1H); ESIMS found for C₁₃H₁₆BNO₅S m/z 292 (M−H₂O)⁺.

Synthesis of 2-((3R,6S)-3-(benzyloxycarbonylamino)-2-hydroxy-1,2-oxaborinan-6-yl)acetic acid 69. An example synthesis of 69 is depicted in Scheme 16 and Example 8.

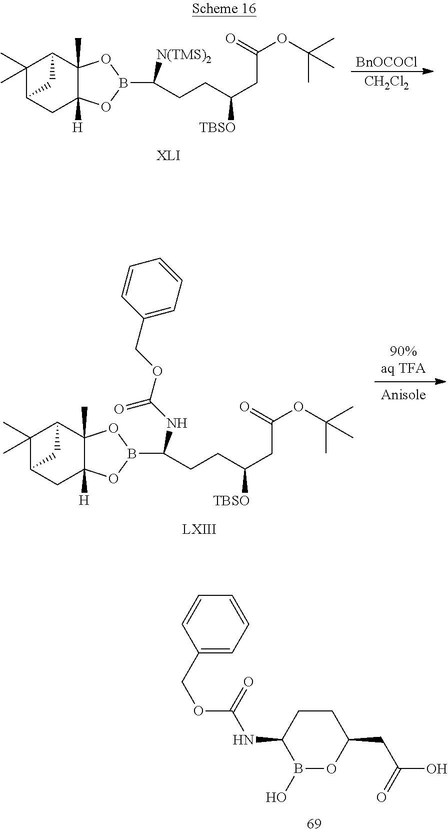

### Example 8

### Step 1

A solution of bis-silyl amide XLI (0.2 mmol) in DCM (5 mL) was cooled to 0° C. and benzyl chloroformate (0.056 mL, 0.4 mmol) was added. Then, the cooling bath was removed and the solution stirred at ambient temperature for 16 h. The reaction was quenched with water and extracted twice with EtOAc. The organic layers were combined, washed with water, brine, dried (Na₂SO₄) and concentrated in vacuo to afford a pale yellow oil as crude product. The residue was chromatographed on a silica column (100% DCM→40% EtOAc/DCM) to afford carbamate LXIII (90 mg, 0.143 mmol, 71.5% yield).

### Step 2

A solution of carbamate LXIII (70 mg, 0.11 mmol) in anisole (5 mL) at 0° C. was treated with pre-cooled 90% aq trifluoroacetic acid (10 mL). The reaction mixture was warmed to room temperature and stirred for 16 h. The mixture was evaporated in vacuo, azeotroped with MeCN (3×5 mL). The residue was sonicated in water (10 mL) and ether (10 mL). The aqueous phase was separated, washed with ether (2×5 mL) and freeze dried to give 2-((3R,6S)-3-(benzyloxycarbonylamino)-2-hydroxy-1,2-oxaborinan-6-yl)acetic acid 69 as a fluffy solid (10 mg, 0.033 mmol, 29.6% yield). ESIMS found for C₁₄H₁₈BNO₆S m/z 289.9 (M−H₂O)⁺.

The following compound is prepared in accordance with the procedure described in the above Example 8.

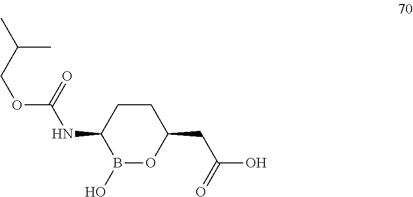 [SMILES: CC(C)COC(=O)N[C@H]1CC[C@@H](CC(=O)O)OB1O]

2-((3R,6S)-2-hydroxy-3-(isobutoxy carbonylamino)-1,2-oxaborinan-6-yl)acetic acid 70 as a off-white solid (20 mg, 0.073 mmol, 27% yield). ¹H NMR (CD₃OD) 6 ppm 0.95 (d, J=7 Hz, 6H), 1.62-1.67 (m, 1H), 1.70-1.75 (m, 2H), 1.87-1.90 (m, 2H), 2.42-2.60 (m, 3H), 3.77-3.86 (m, 2H), 4.35-4.38 (m, 1H); ESIMS found for C₁₁H₂₀BNO₆S m/z 256 (M−H₂O)⁺.

Synthesis of 2-((3R,6S)-2-hydroxy-3-(phenylsulfonamido)-1,2-oxaborinan-6-yl)acetic acid 71. An example synthesis of 71 is depicted in Scheme 17 and Example 9.

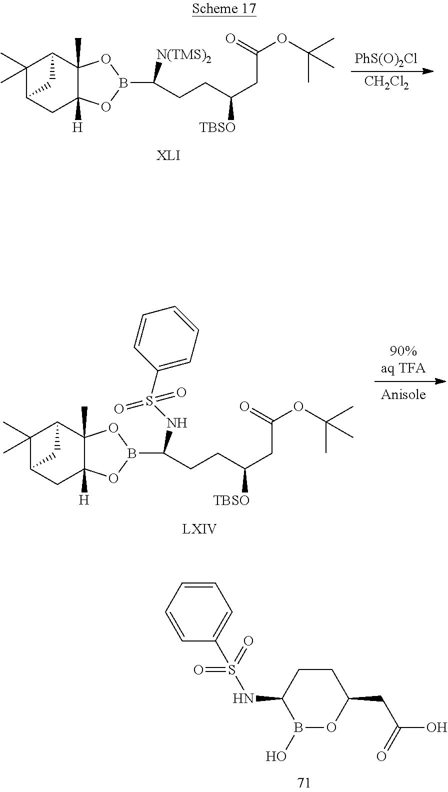

### Example 9

### Step 1-2

Prepared in accordance with the procedure described in the above Steps 1-2 of Example 8.

Off-white solid (30 mg, 0.096 mmol, 43% yield). ¹H NMR (CD₃OD) δ ppm 157-1.83 (series of m, 4H), 2.49-2.71 (series of m, 3H), 4.35-4.89 (m, 1H), 7.51-7.59 (m, 3H), 7.85-7.89 (m, 2H); ESIMS found for C₁₂H₁₆BNO₆S m/z 296J (M−H₂O)⁺.

Synthesis of 2-((3R,6S)-2-hydroxy-3-(3-phenylureido)-1,2-oxaborinan-6-yl)acetic acid 72. An example synthesis of 72 is depicted in Scheme 18 and Example 10.

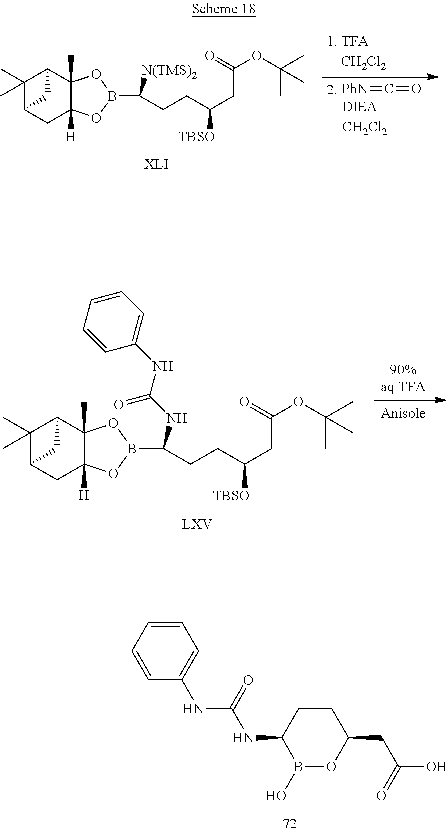

### Example 10

### Step 1

To a solution of bis-silyl amide XLI (0.2 mmol) in DCM (5 mL) at 0° C. was added a solution of TFA in hexane (0.6 mmol). The reaction was stirred at 0° C. for 20 min before adding phenyl isocayanate (0.04 mL, 0.4 mmol) followed by N,N-diisopropylethylamine (0.18 mL, 1 mmol). The cooling bath was then removed and the solution was stirred at ambient temperature for 16 h. The reaction was quenched with water and extracted twice with EtOAc. The organic layers were combined, washed with water, brine, dried (Na₂SO₄) and concentrated in vacuo to afford a pale yellow oil as crude product. The residue was chromatographed on a silica column (100% DCM-25% EtOAc/DCM) to afford the pure urea (50 mg, 0.081 mmol, 40.7% yield).

### Step 2

Deprotection was performed following the procedure described above in step 2 of example 8 to give 2-((3R,6S)-2-hydroxy-3-(3-phenylureido)-1,2-oxaborinan-6-yl)acetic acid 72 as a white solid (20 mg, 0.068 mmol, 86% yield). ¹H NMR (CD₃OD) δ ppm 1.24-1.31 (m, 1H), 1.56-1.64 (m, 2H) 1.78-1.81 (m, 1H), 2.36-2.40 (dd, J=15 Hz, J=6 Hz, 1H), 2.46-2.58 (dd, J=13 Hz, J=7 Hz, 1H), 2.68-2.71 (m, 1H), 4.07-4.12 (m, 1H), 7.15-7.18 (m, 1H), 7.34-7.37 (m, 4H); ESIMS found for C₁₃H₁₇BN₂O₅ m/z 275.1 (M−H₂O)⁺.

Illustrative compounds of Formula (I) are shown in Table 1. Some structures are shown with defined configurations at selected stereocenters but the shown stereochemistries are not meant to be limiting and all possible stereoisomers of the shown structures are to be considered encompassed herein. Compounds of any absolute and relative configurations at the stereocenters as well as mixtures of enantiomers and diastereoisomers of any given structure are also encompassed herein.

TABLE 1

| Example | Structure |
|---|---|
| 1 | 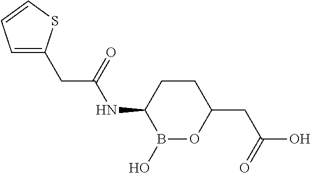 [SMILES: O=C(O)CC1CC[C@H](NC(=O)Cc2cccs2)B(O)O1] |
| 2 | 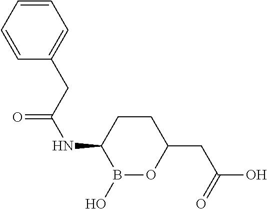 [SMILES: O=C(O)CC1CC[C@H](NC(=O)Cc2ccccc2)B(O)O1] |
| 3 | 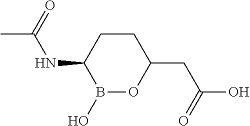 |
| 4 | 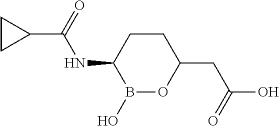 [SMILES: O=C(O)CC1CC[C@H](NC(=O)C2CC2)B(O)O1] |
| 5 | 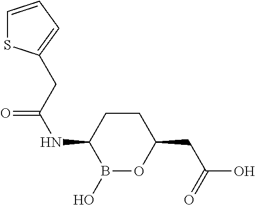 [SMILES: O=C(O)C[C@@H]1CC[C@H](NC(=O)Cc2cccs2)B(O)O1] |
| 6 | 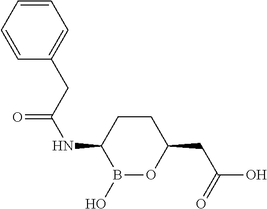 [SMILES: O=C(O)C[C@@H]1CC[C@H](NC(=O)Cc2ccccc2)B(O)O1] |
| 7 | 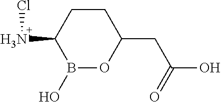 |
| 8 | 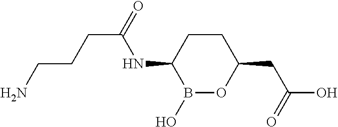 [SMILES: NCCCC(=O)N[C@H]1CC[C@@H](CC(=O)O)OB1O] |
| 9 | 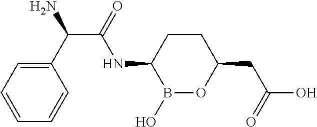 [SMILES: N[C@@H](C(=O)N[C@H]1CC[C@@H](CC(=O)O)OB1O)c1ccccc1] |
| 10 | 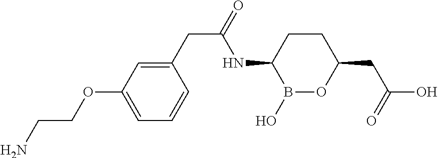 [SMILES: NCCOc1cccc(CC(=O)N[C@H]2CC[C@@H](CC(=O)O)OB2O)c1] |
| 11 | 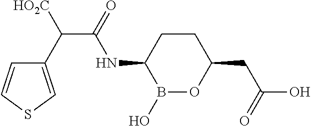 [SMILES: O=C(O)C[C@@H]1CC[C@H](NC(=O)C(C(=O)O)c2ccsc2)B(O)O1] |
| 12 | 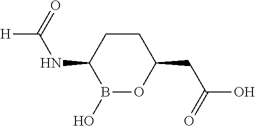 [SMILES: O=CN[C@H]1CC[C@@H](CC(=O)O)OB1O] |
| 13 | 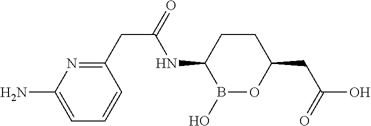 [SMILES: Nc1cccc(CC(=O)N[C@H]2CC[C@@H](CC(=O)O)OB2O)n1] |
| 14 | 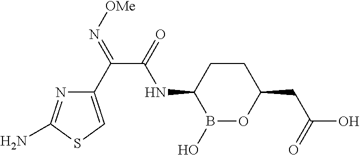 [SMILES: CO/N=C(\C(=O)N[C@H]1CC[C@@H](CC(=O)O)OB1O)c1csc(N)n1] |
| 15 | 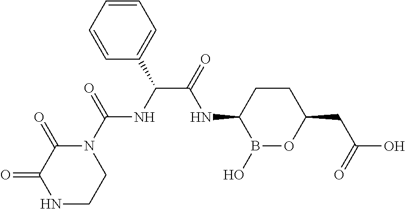 [SMILES: O=C(O)C[C@@H]1CC[C@H](NC(=O)[C@H](NC(=O)N2CCNC(=O)C2=O)c2ccccc2)B(O)O1] |
| 16 | 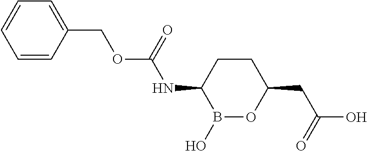 [SMILES: O=C(O)C[C@@H]1CC[C@H](NC(=O)OCc2ccccc2)B(O)O1] |
| 17 | 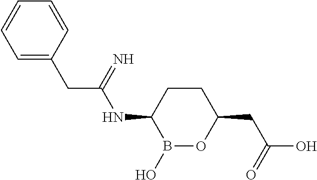 [SMILES: N=C(Cc1ccccc1)N[C@H]1CC[C@@H](CC(=O)O)OB1O] |
| 18 | 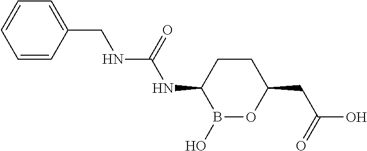 [SMILES: O=C(O)C[C@@H]1CC[C@H](NC(=O)NCc2ccccc2)B(O)O1] |
| 19 | 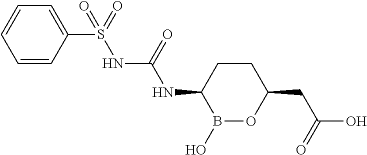 [SMILES: O=C(O)C[C@@H]1CC[C@H](NC(=O)NS(=O)(=O)c2ccccc2)B(O)O1] |
| 20 | 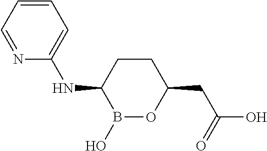 [SMILES: O=C(O)C[C@@H]1CC[C@H](Nc2ccccn2)B(O)O1] |
| 21 | 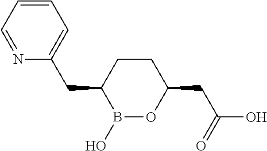 [SMILES: O=C(O)C[C@@H]1CC[C@H](Cc2ccccn2)B(O)O1] |
| 22 | 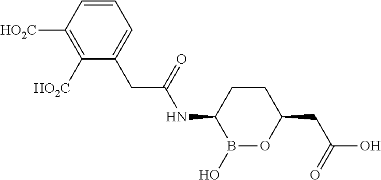 [SMILES: O=C(O)C[C@@H]1CC[C@H](NC(=O)Cc2cccc(C(=O)O)c2C(=O)O)B(O)O1] |
| 23 | 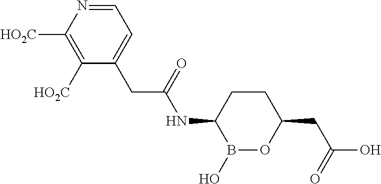 [SMILES: O=C(O)C[C@@H]1CC[C@H](NC(=O)Cc2ccnc(C(=O)O)c2C(=O)O)B(O)O1] |
| 24 | 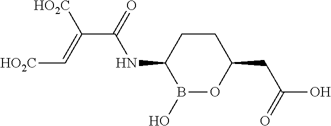 [SMILES: O=C(O)/C=C(\C(=O)O)C(=O)N[C@H]1CC[C@@H](CC(=O)O)OB1O] |
| 25 | 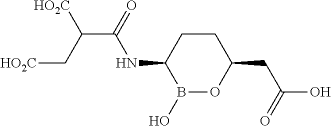 [SMILES: O=C(O)CC(C(=O)O)C(=O)N[C@H]1CC[C@@H](CC(=O)O)OB1O] |
| 26 | 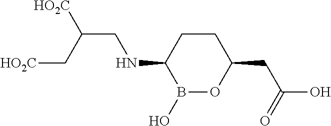 [SMILES: O=C(O)CC(CN[C@H]1CC[C@@H](CC(=O)O)OB1O)C(=O)O] |
| 27 | 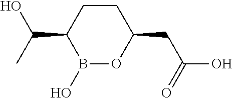 |
| 28 | 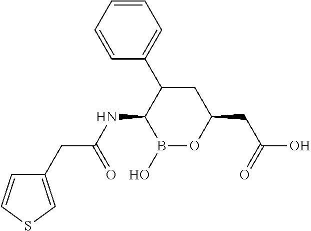 [SMILES: O=C(O)C[C@@H]1CC(c2ccccc2)[C@H](NC(=O)Cc2ccsc2)B(O)O1] |
| 29 | 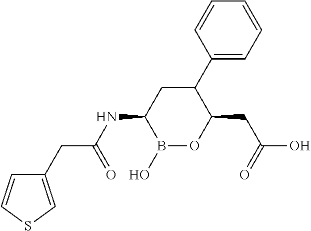 [SMILES: O=C(O)C[C@H]1OB(O)[C@@H](NC(=O)Cc2ccsc2)CC1c1ccccc1] |
| 30 | 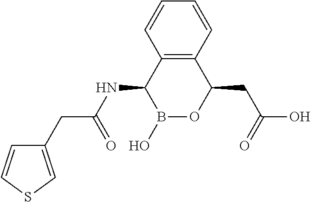 [SMILES: O=C(O)C[C@H]1OB(O)[C@@H](NC(=O)Cc2ccsc2)c2ccccc21] |
| 31 | 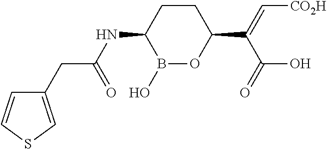 [SMILES: O=C(O)/C=C(\C(=O)O)[C@@H]1CC[C@H](NC(=O)Cc2ccsc2)B(O)O1] |
| 32 | 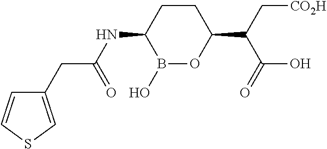 [SMILES: O=C(O)CC(C(=O)O)[C@@H]1CC[C@H](NC(=O)Cc2ccsc2)B(O)O1] |
| 33 | 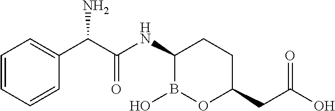 [SMILES: N[C@H](C(=O)N[C@H]1CC[C@@H](CC(=O)O)OB1O)c1ccccc1] |
| 34 | 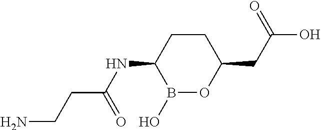 [SMILES: NCCC(=O)N[C@H]1CC[C@@H](CC(=O)O)OB1O] |
| 35 | 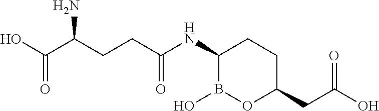 [SMILES: N[C@@H](CCC(=O)N[C@H]1CC[C@@H](CC(=O)O)OB1O)C(=O)O] |
| 36 | 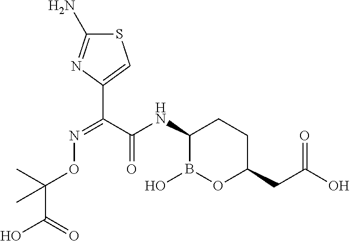 [SMILES: CC(C)(O/N=C(\C(=O)N[C@H]1CC[C@@H](CC(=O)O)OB1O)c1csc(N)n1)C(=O)O] |
| 37 | 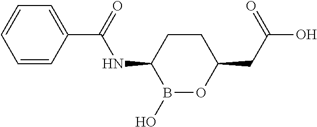 [SMILES: O=C(O)C[C@@H]1CC[C@H](NC(=O)c2ccccc2)B(O)O1] |
| 38 | 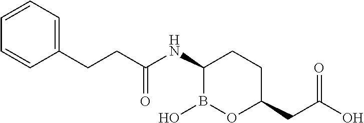 [SMILES: O=C(O)C[C@@H]1CC[C@H](NC(=O)CCc2ccccc2)B(O)O1] |
| 39 | 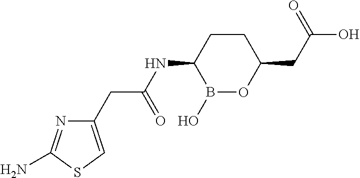 [SMILES: Nc1nc(CC(=O)N[C@H]2CC[C@@H](CC(=O)O)OB2O)cs1] |
| 40 | 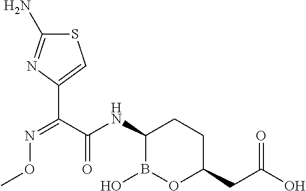 [SMILES: CO/N=C(\C(=O)N[C@H]1CC[C@@H](CC(=O)O)OB1O)c1csc(N)n1] |
| 41 | 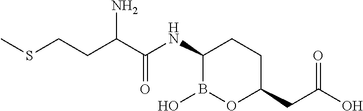 [SMILES: CSCCC(N)C(=O)N[C@H]1CC[C@@H](CC(=O)O)OB1O] |
| 42 | 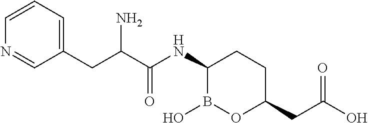 [SMILES: NC(Cc1cccnc1)C(=O)N[C@H]1CC[C@@H](CC(=O)O)OB1O] |
| 43 | 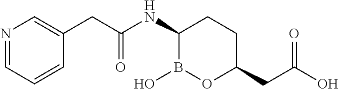 [SMILES: O=C(O)C[C@@H]1CC[C@H](NC(=O)Cc2cccnc2)B(O)O1] |
| 44 | 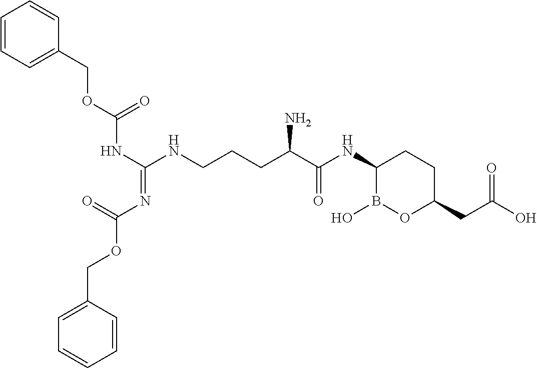 [SMILES: N[C@H](CCCN/C(=N/C(=O)OCc1ccccc1)NC(=O)OCc1ccccc1)C(=O)N[C@H]1CC[C@@H](CC(=O)O)OB1O] |
| 45 | 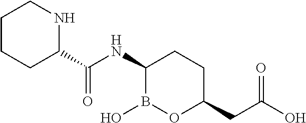 |
| 46 | 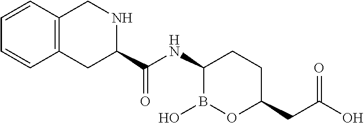 [SMILES: O=C(O)C[C@@H]1CC[C@H](NC(=O)[C@H]2Cc3ccccc3CN2)B(O)O1] |
| 47 | 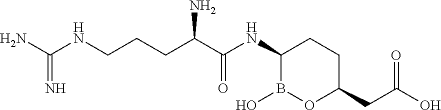 [SMILES: N=C(N)NCCC[C@@H](N)C(=O)N[C@H]1CC[C@@H](CC(=O)O)OB1O] |
| 48 | 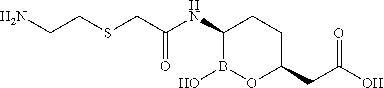 [SMILES: NCCSCC(=O)N[C@H]1CC[C@@H](CC(=O)O)OB1O] |
| 49 | 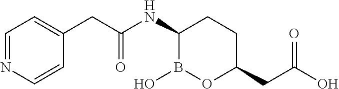 [SMILES: O=C(O)C[C@@H]1CC[C@H](NC(=O)Cc2ccncc2)B(O)O1] |
| 50 | 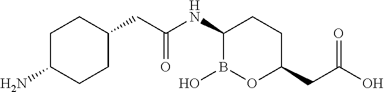 [SMILES: N[C@H]1CC[C@@H](CC(=O)N[C@H]2CC[C@@H](CC(=O)O)OB2O)CC1] |
| 51 | 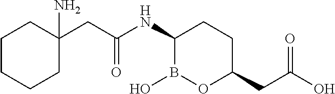 [SMILES: NC1(CC(=O)N[C@H]2CC[C@@H](CC(=O)O)OB2O)CCCCC1] |
| 52 | 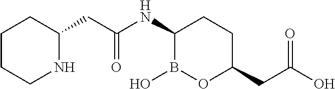 [SMILES: O=C(O)C[C@@H]1CC[C@H](NC(=O)C[C@H]2CCCCN2)B(O)O1] |
| 53 | 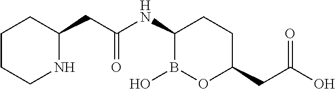 [SMILES: O=C(O)C[C@@H]1CC[C@H](NC(=O)C[C@@H]2CCCCN2)B(O)O1] |
| 54 | 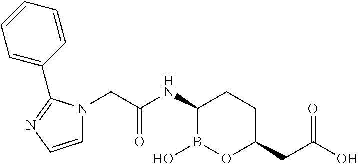 [SMILES: O=C(O)C[C@@H]1CC[C@H](NC(=O)Cn2ccnc2-c2ccccc2)B(O)O1] |
| 55 | 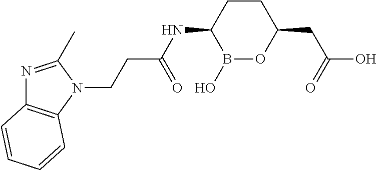 [SMILES: Cc1nc2ccccc2n1CCC(=O)N[C@H]1CC[C@@H](CC(=O)O)OB1O] |
| 56 | 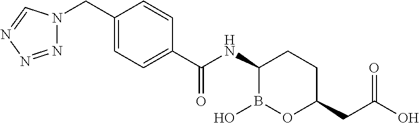 [SMILES: O=C(O)C[C@@H]1CC[C@H](NC(=O)c2ccc(Cn3cnnn3)cc2)B(O)O1] |
| 57 | 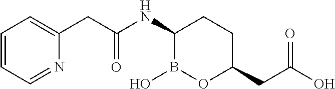 [SMILES: O=C(O)C[C@@H]1CC[C@H](NC(=O)Cc2ccccn2)B(O)O1] |
| 58 | 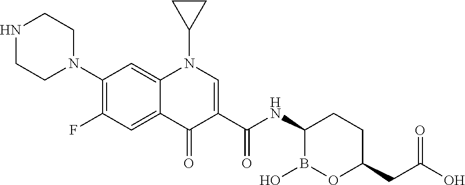 [SMILES: O=C(O)C[C@@H]1CC[C@H](NC(=O)c2cn(C3CC3)c3cc(N4CCNCC4)c(F)cc3c2=O)B(O)O1] |
| 59 | 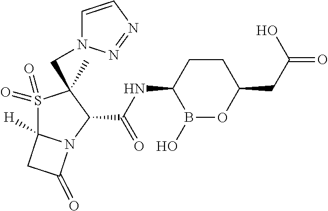 [SMILES: C[C@]1(Cn2ccnn2)[C@H](C(=O)N[C@H]2CC[C@@H](CC(=O)O)OB2O)N2C(=O)C[C@H]2S1(=O)=O] |
| 60 | 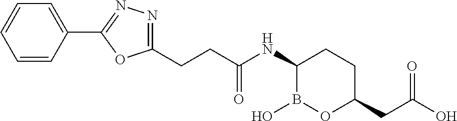 [SMILES: O=C(O)C[C@@H]1CC[C@H](NC(=O)CCc2nnc(-c3ccccc3)o2)B(O)O1] |
| 61 | 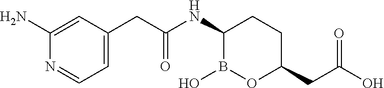 [SMILES: Nc1cc(CC(=O)N[C@H]2CC[C@@H](CC(=O)O)OB2O)ccn1] |
| 62 | 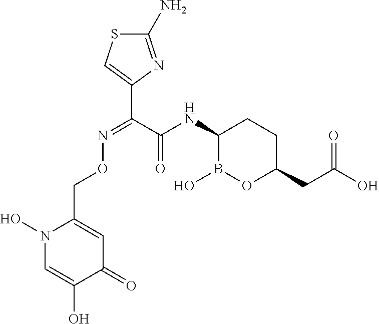 [SMILES: Nc1nc(/C(=N/OCc2cc(=O)c(O)cn2O)C(=O)N[C@H]2CC[C@@H](CC(=O)O)OB2O)cs1] |
| 63 | 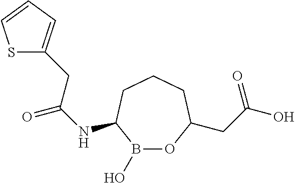 [SMILES: O=C(O)CC1CCC[C@H](NC(=O)Cc2cccs2)B(O)O1] |
| 64 | 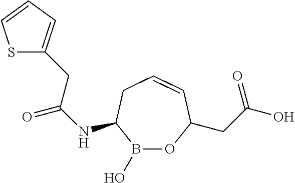 [SMILES: O=C(O)CC1C=CC[C@H](NC(=O)Cc2cccs2)B(O)O1] |
| 65 | 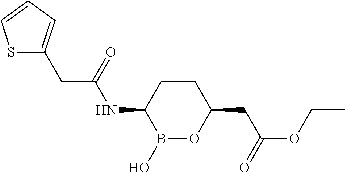 [SMILES: CCOC(=O)C[C@@H]1CC[C@H](NC(=O)Cc2cccs2)B(O)O1] |
| 66 | 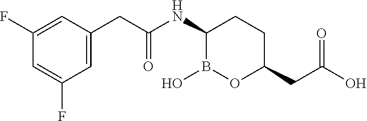 [SMILES: O=C(O)C[C@@H]1CC[C@H](NC(=O)Cc2cc(F)cc(F)c2)B(O)O1] |
| 67 | 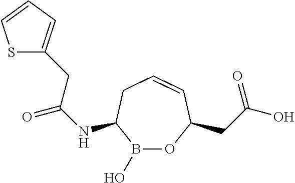 [SMILES: O=C(O)C[C@@H]1C=CC[C@H](NC(=O)Cc2cccs2)B(O)O1] |
| 68 | 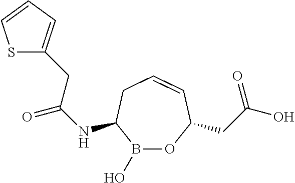 [SMILES: O=C(O)C[C@H]1C=CC[C@H](NC(=O)Cc2cccs2)B(O)O1] |
| 69 | 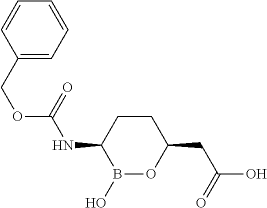 [SMILES: O=C(O)C[C@@H]1CC[C@H](NC(=O)OCc2ccccc2)B(O)O1] |
| 70 | 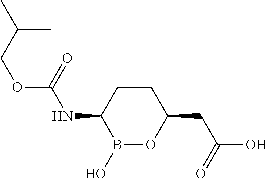 [SMILES: CC(C)COC(=O)N[C@H]1CC[C@@H](CC(=O)O)OB1O] |
| 71 | 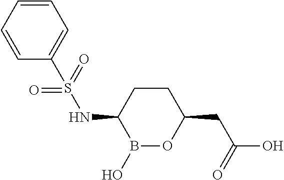 [SMILES: O=C(O)C[C@@H]1CC[C@H](NS(=O)(=O)c2ccccc2)B(O)O1] |
| 72 | 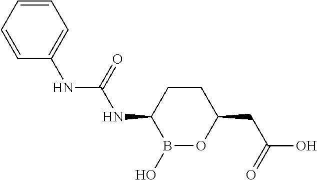 [SMILES: O=C(O)C[C@@H]1CC[C@H](NC(=O)Nc2ccccc2)B(O)O1] |

### Formula (II)

Compounds of formula (II) where R^{1α} is an acylamino group, Z^{α} is —O— or —S— and X^{α} is a carboxylic acid can be prepared as depicted in Scheme 1A. In the following schemes only, that is Schemes 1A-7A, an element that is denoted in the text with a superscript alpha (^{α}) is represented in the corresponding diagramed element without the superscript alpha (^{α}). For example, in Scheme 1A, structure IV^{α} in the text corresponds to diagrammed structure IV.

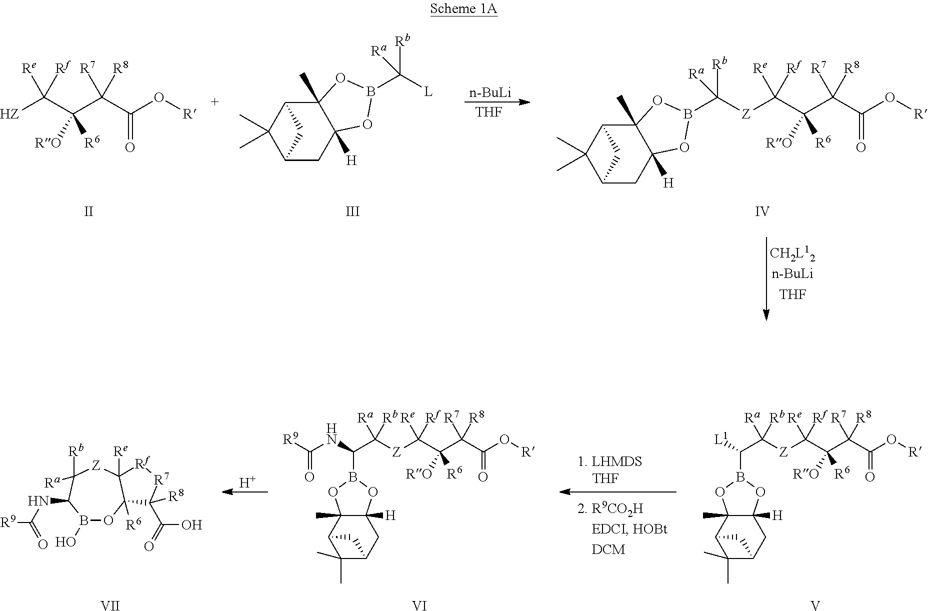

Compounds of structure IV^{α} where Z^{α} is —O— can be made via lithium alkoxide formation of alcohol II^{α} (Z=—O—) [J. Org. Chem. (2010), 75, 3953-3957; WO0587700] and reaction with halomethyleneboronate esters [Tetrahedron (2005), 61, 4427-4436; J. Am. Chem. Soc. (1990), 112, 3964-3969]. Compounds where Z^{α} is —S— in IV^{α} may be attained via thiol version of II^{α} (Z^{α}=—S—). Such thiol compounds can be made from the corresponding alcohol by variety of known procedures (Tetrahedron: Asymmetry (1993), 4, 361-8). Homologation of IV^{α} to V^{α} where L^{1α} is chloro is achieved via Matteson reaction conditions with good stereocontrol [WO0946098; Tetrahedron (1998), 54, 10555-10607]. The chloro derivative of V^{α} can be utilized to introduce a substituted amine group at the alpha-position of boronate. Stereospecific substitution with hexamethyldisilazane gives the corresponding bis(trimethylsilyl) amide which may be reacted in situ with an acid chloride to result directly in analogs of structure VI^{α}. Such analogs of VI^{α} can also be made via coupling of the bis-TMS amine with commercially available carboxylic acids under typical amide coupling conditions (e.g., carbodiimide or HATU coupling). Simultaneous deprotection of the pinane ester, acid sensitive OR′^{α} and OR″^{α} groups and concomitant cyclization are achieved by heating with dilute HCl, affording the desired cyclic boronate derivatives of structure VII^{α}. This transformation may also be achieved by treatment with BCl₃ or BBr₃ (WO09064414). Alternatively, the deprotection may be attained via trans-esterification with isobutyl boronic acid in presence of dilute HCl (WO09064413) or via other known methods [J. Org. Chem. (2010), 75, 468-471].

Compounds of structure IX^{α} where R^{1α} of Formula II is an alkyl, aralkyl or aminoaryl group may be made from intermediate V^{α} as shown in Scheme 2A.

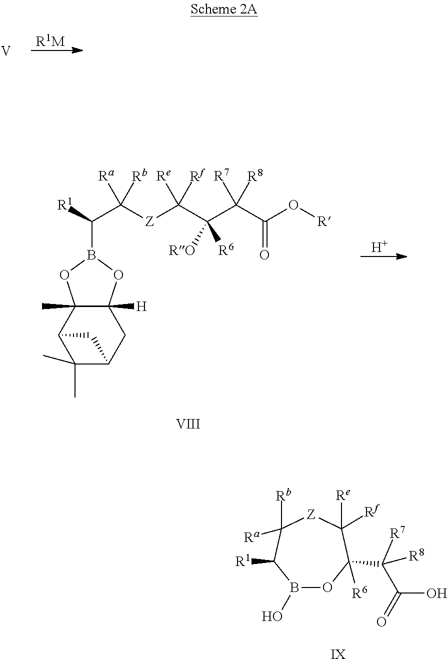

Compounds of structure IX^{α} may be made from intermediate V^{α}, where L^{1α} preferably an iodo, or bromo group [J. Organomet. Chem. (1992), 431, 255-70]. Such bromo derivatives may be made as analogously to the chloro compounds of Scheme 1A, utilizing dibromomethane [J. Am. Chem. Soc. (1990), 112, 3964-969]. Displacement of the bromo group in V^{α} can be achieved by α-alkoxy substituted alkyllithium agents [J. Am. Chem. Soc. (1989), 111, 4399-402; J. Am. Chem. Soc. (1988), 110, 842-53] or organomagnesium reagents (WO0946098) or by the sodium salt of alkyl or aryl carbamate derivatives [J. Org. Chem. (1996), 61, 7951-54], resulting in VIII^{α}. Deprotection and cyclization of VIII^{α} to afford IX^{α} may be achieved under the conditions described in Scheme 1A.

Compounds of formula II where R^{1α} is an acylamino group, Z^{α} is —N[C(═O)R^{9α}]— and X^{α} is a carboxylic acid can be prepared as depicted in Scheme 3A.

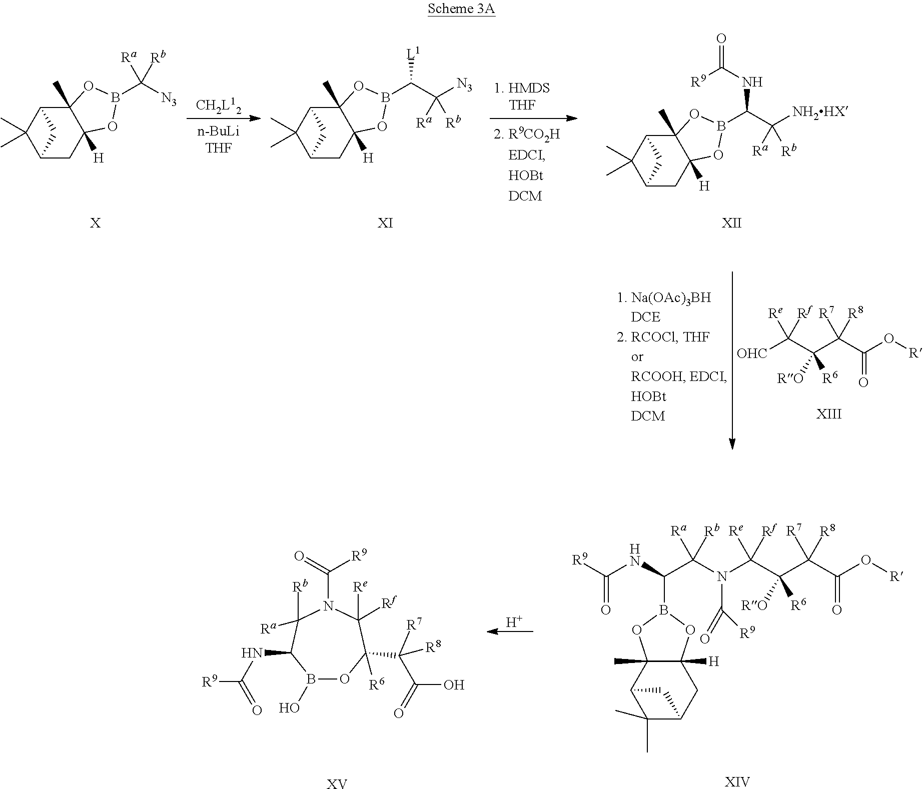

Enantiomerically pure 1,2-diamino-propyl boronate derivatives of structure XII^{α} are made utilizing Matteson protocol as described above, starting from azidomethylene boronate of structure X^{α} [Organometallics (1996) 15, 152-163] via halomethylene insertion product XI^{α} [J. Organomet. Chem. (2008), 693, 2258-2262]. Compounds of structure XII^{α} can be further transformed to XIV^{α} by well known reductive amination transformation [J. Org. Chem. (1996), 61, 3849-3862] with carbonyl intermediates such as XIII^{α}, followed by installation of R^{9α}CO— group on the resulting amine. Cyclic boronates of structure XV^{α} are attained from intermediate XIV^{α} by simultaneous deprotection and cyclization in acid hydrolysis conditions described in Scheme 1A. A sequential deprotection and cyclization protocol may be followed where —OR′^{α} and —OR″^{α} a of structure XIV^{α} are not acid sensitive protective groups.

Compounds of formula II where R^{1α} is an acylamino group, G^{1α} is null, G^{2α} is a substituted carbonyl alkyl group, Z^{α} is —N[C(═O)R^{9α}]— and X^{α} is a carboxylic acid can be prepared as depicted in Scheme 4A.

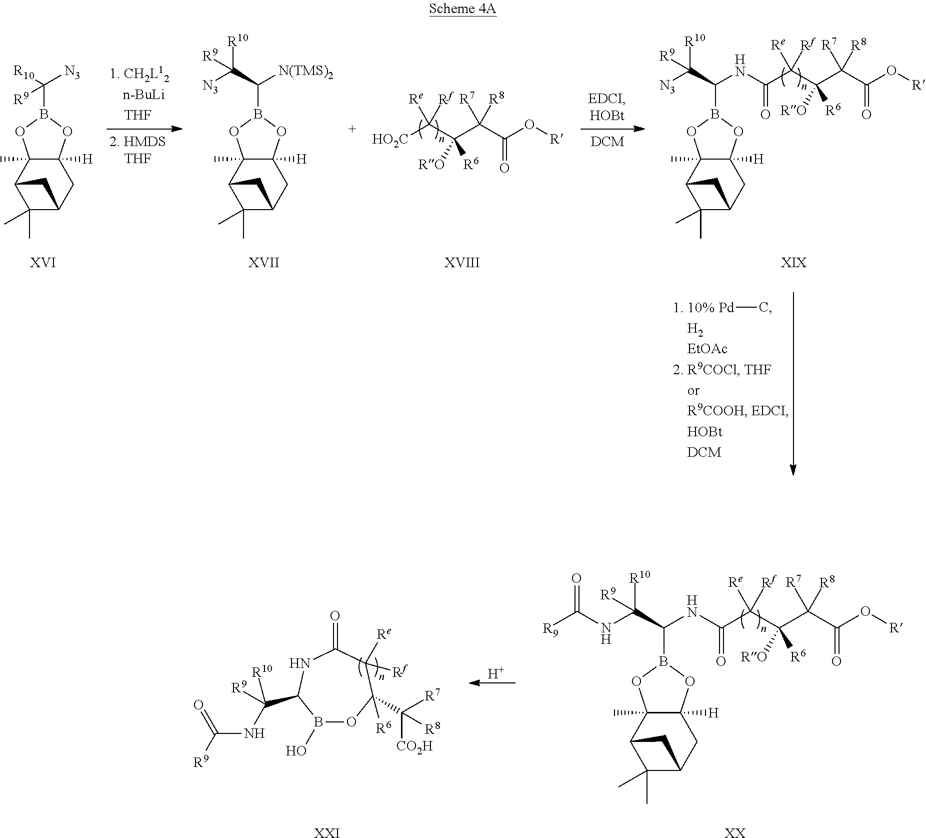

Bis-trimethylsilyl amino intermediate XVII^{α} may be made as described above in Scheme 3A starting from azidomethylene boronate XVII [J. Organomet. Chem. (2008), 693, 2258-2262]. These derivatives as XVII^{α} can be directly utilized in amide coupling reactions with carboxylic acid intermediates of structure XVIII^{α}. Such intermediates of structure XVIII^{α} with suitable protective groups, where n is 0 or 1 can be obtained by procedures described earlier in both enantiomeric forms [WO0691771, J. Org. Chem. (1989), 54, 2085-2091]. Resulting azido-amides of structure XIX^{α} from amide coupling reaction can be then further transformed to bis-amide XX^{α}. Such transformation may be achieved by reduction via hydrogenation conditions in presence of a palladium catalyst followed by acylation of the resulting amine to XX^{α}. Final deprotection-cyclization to compounds of formula XXI^{α} may be achieved in single step or sequentially based on the choice of —OR′^{α} and —OR″^{α} groups of XVIII^{α} as described above.

Compounds of formula XXVII^{α} and XXVIII^{α} can be made following the sequence depicted in Scheme 5A,

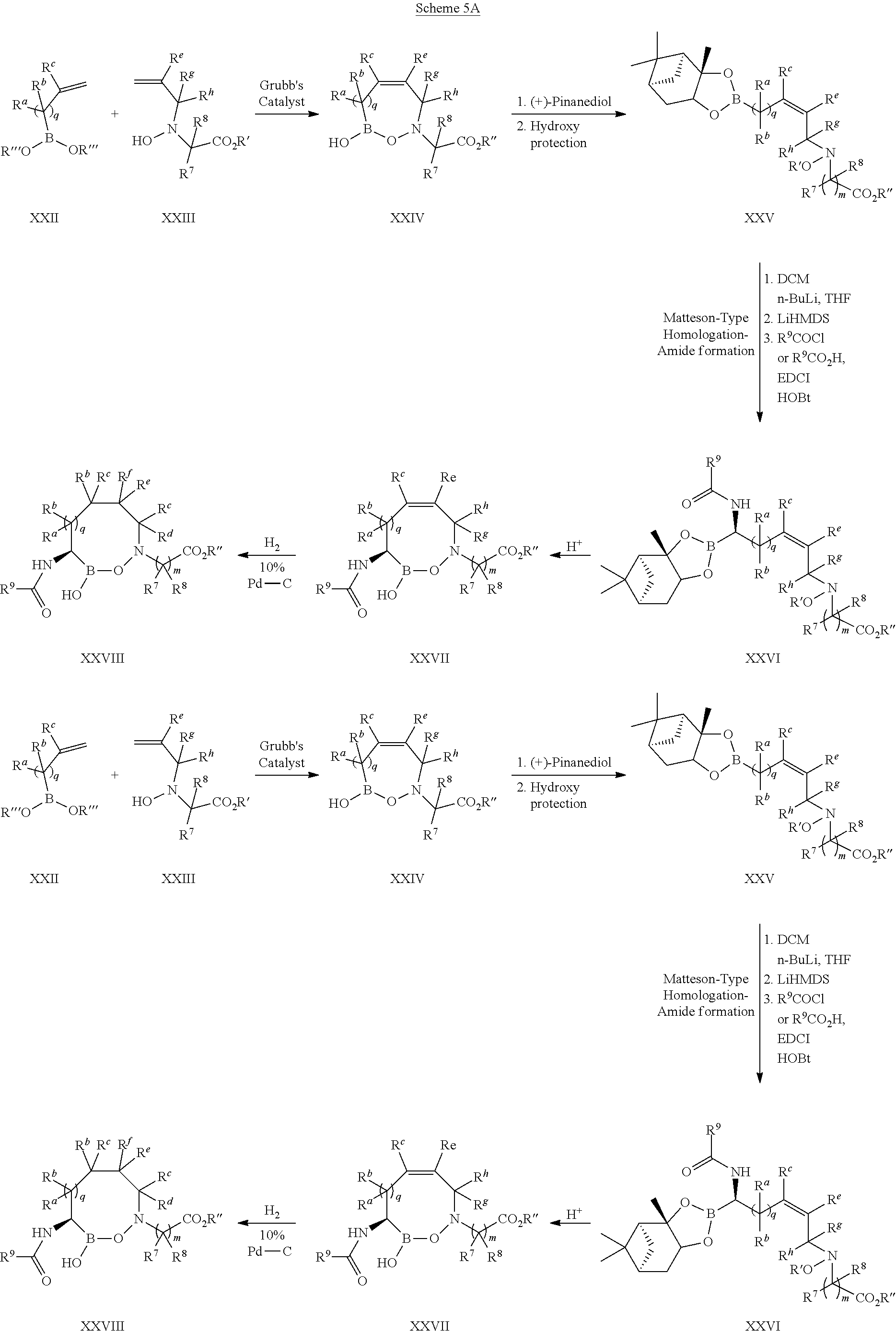

Ring-Closing Metathesis reaction (RCM) with commercially available boronated olefins (XXII^{α}) and olefin substituted hydroxylamine esters (XXIII^{α}) result in cyclic boronates of formula XXIV^{α} [Angew. Chem. Int. Ed. (2002), 41, 152-154]. Such substituted hydroxylamine acetic acid esters (XXIII^{α}) may be made by alkenylation of known intermediates [J. Org. Chem. (2005), 70, 10494-10501]. Cyclic boronates (XXIV^{α}) undergo ready esterification with chiral pinane diol of choice to give required Matteson reaction precursors, upon protection of the resulting alcohol with groups such as t-butyldimethylsilyl- or benzyl or trityl. Matteson-Type homologation followed by amide formation result in compounds of formula XXVI^{α} with high stereoselectivity, as described above in Scheme 1A. Acid mediated hydrolysis of compounds of XXVI^{α} upon deprotection give cyclic boronate (XXVII^{α}). Double bond substitution of XXVII^{α} can be further modified to other analogs or to a saturated cyclic boronate (XXVIII^{α}) by catalytic hydrogenation. The above sequence can be utilized to make 7- or 8-membered rings with double bond by varying XXII^{α} where q^{α} is 0 or 1.

Compounds of formula II where R^{1α} is an acylamino group, G^{1α} is null, G^{2α} is a substituted alkyl carbonyl group, Z^{α} is —C(R^{9α}R^{10α}) Y^{α} is N and X^{α} is a carboxylic acid can be prepared as depicted in Scheme 6A.

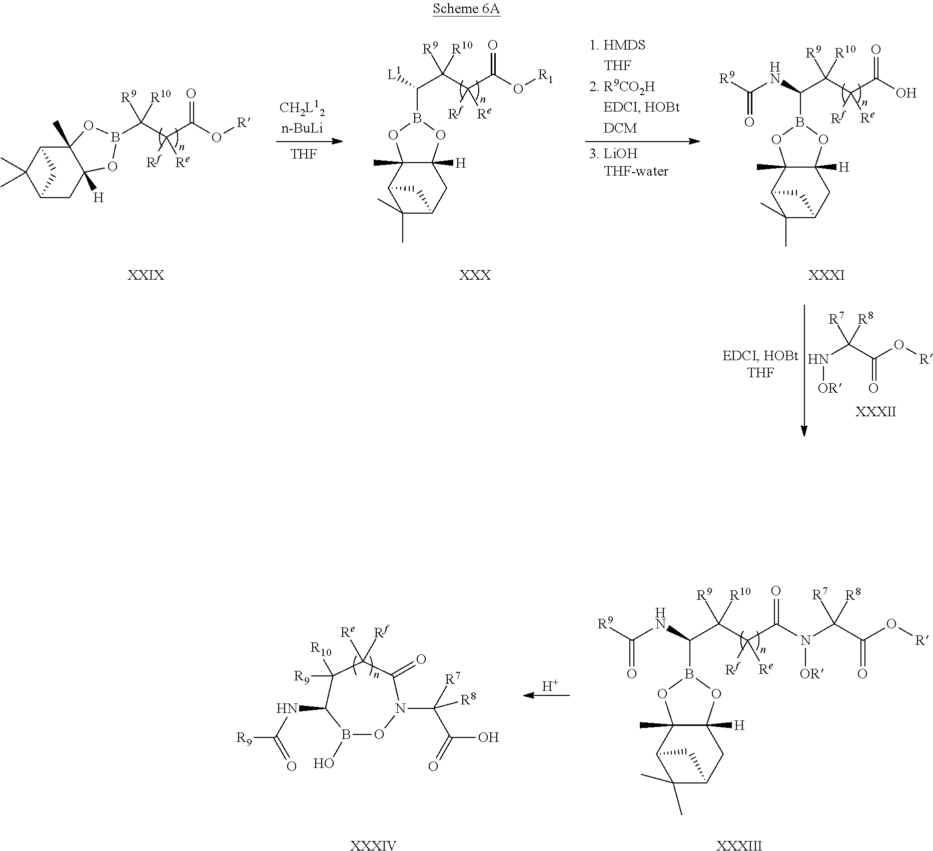

Synthesis of compounds of structure XXXIV^{α} can be attained starting from known intermediates of structure XXX^{α} (n^{α} is 0 or 1), in racemic or enatiomerically pure form. Matteson-Type homologation of XXIX [Tetrahedron Lett. (1987), 28, 4499-4502] followed by amination and amide formation result in ester derivative of XXXI^{α}. Such ester can hydrolysed under mild conditions to give the carresponding carboxylic acid (XXXI^{α}). Alternatively, such carboxylic acids can also be made in racemic form via azido substitution sequence [U.S. Pat. No. 6,586,615; J. Org. Chem. (2001), 66, 6375-6380]. Amide formation of substituted and β-hydroxylamine esters with suitable protective groups (—OR′^{α} as silyloxy or benzyloxy) result in the formation of compounds of structure XXXIII^{α}[J. Chem. Soc., Perkin Trans 1, (1989), 2, 235-9]. Cyclic boronate compounds of formula XXXIV^{α} can be obtained by deprotection-cyclization of compounds of formula XXXIII^{α}, in single step or sequentially based on the choice of —OR′^{α} and —OR″^{α} groups. Enantiomercally pure compounds of XXXIV^{α} can also be attained by chiral chromatography of the racemic precursors or the final compounds.

The syntheses of compounds of formulae VII^{α}, XIX^{α}, XV^{α} and XXI^{α} in the above sequences are described for trans-isomers. These methods can also be utilized to make cis-isomers in enantiomerically pure form by starting (as in Schemes 1A to 4A) with corresponding enantiomer.

Compounds of formula II where X^{α} is a carboxylic acid isostere can be prepared following the protocols described earlier in literature [J. Med. Chem. (2011), 54, 2529-2591].

### Illustrative Compound Examples

Synthesis of 2-((3R)-2-hydroxy-3-(2-(thiophen-2-yl)acetamido)-1,5,2-dioxaborepan-7-yl)acetic acid. An example synthesis of 1^{α} is depicted in Scheme 7A and Example 10.

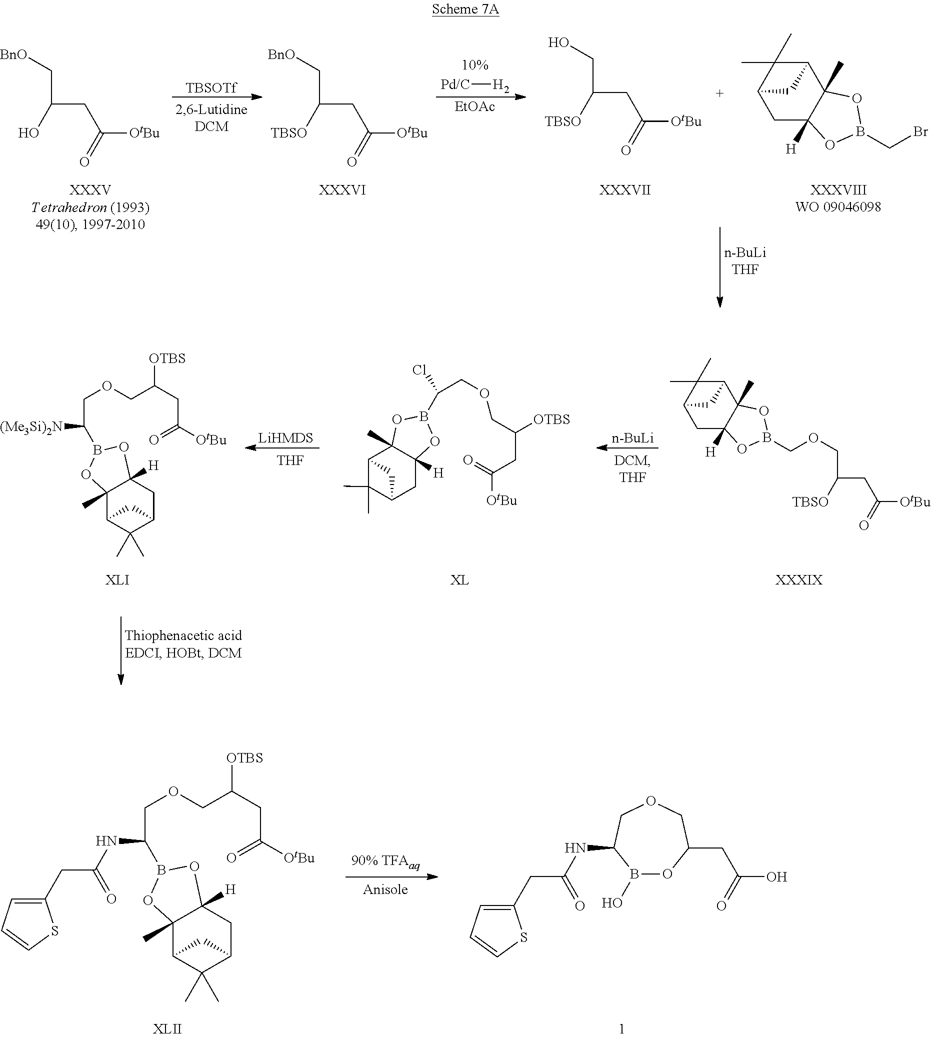

### Example 10

### Step 1

To a solution of tert-butyl-4-(benzyloxy)-3-hydroxybutanoate XXXV^{α} [Tetrahedron (1993), 49(10), 1997-2010] (2.3 g, 8.84 mmol) in DCM (100 mL) at 0° C. was added 2,6-lutidine (3.07 mL, 26.52 mmol) and TBSOTf (4 mL, 4. 17.68 mmol). After stirring for 16 h at 0° C., the reaction was diluted with EtOAc (400 mL). The mixture was washed with 1N HCl, saturated aq NaHCO₃, water and dried. The extract was dried (MgSO4) and concentrated under reduced pressure. Purification of the crude product by column chromatography (100% hexane→25% EtOAc/hexane) afforded tert-butyl 4-(benzyloxy)-3-(tert-butyldimethylsilyloxy)butanoate XXXVI^{α} (3.1 g, 8.15 mmol, 92.1% yield) as a colorless oil.

### Step 2

To a solution of tert-butyl 4-(benzyloxy)-3-(tert-butyldimethylsilyloxy) butanoate XXXVI^{α} (3.1 g, 8.15 mmol) in EtOAc (200 mL) under a nitrogen atmosphere was added 10% palladium on carbon (600 mg). The reaction flask was evacuated and then charged with a balloon of hydrogen. The reaction mixture was then stirred at room temperature for 16 h before being filtered through Celite. The filtrate was then concentrated under reduced pressure. Purification of the crude product by column chromatography (100% DCM→50% EtOAc/DCM) afforded tert-butyl 3-(tert-butyldimethylsilyloxy)-4-hydroxybutanoate XXXVII^{α} (2.1 g, 7.22 mmol, 88.7% yield) as a colorless oil.

### Step 3

To a solution of tert-butyl 3-(tert-butyldimethylsilyloxy)-4-hydroxybutanoate XXXVI^{α} (1 g mL, 3.44 mmol) in anhydrous THF (15 mL) at −78° C. with an acetone/dry ice bath was added n-BuLi (2.5 M in hexanes, 1.38 mL, 3.44 mmol) slowly. The mixture was stirred at −78° C. for 15 min. DMSO (0.25 mL, 3.44 mmol) was added dropwise followed by bromide intermediate XXXVIII^{α} (WO 09046098) (937 g, 3.44 mmol). The reaction was allowed to reach room temperature slowly and then was heated at 50° C. overnight. The reaction mixture was then diluted with diethyl ether (200 mL) and washed with aqueous HCl (0.6 N, 200 mL). The aqueous layer was re-extracted with diethyl ether (2×100 mL). The organic layers were combined and concentrated in vacuo. Purification of the crude oil by flash chromatography (100% hexane→25% EtOAc/hexane) afforded alkoxy intermediate XXXIX^{α} (460 mg, 0.95 mmol, 27.7% yield) as a colorless oil.

### Step 4

To a solution of DCM (0.13 mL, 2.15 mmol) in THF (5 mL) at −100° C. was added 2.5 M n-butyl lithium in hexane (0.64 mL, 1.61 mmol) slowly under nitrogen and down the inside wall of the flask, maintaining the temperature below −90° C. The resulting white precipitate was stirred for 30 minutes before the addition of alkoxy intermediate XXXIV from step 3 (520 mg, 1.078 mmol) in THF (2 mL) at −90° C. Zinc chloride (3.77 mL, 1M in diethyl ether, 3.77 mmol) was then added to the reaction mixture at −90° C. and then the reaction was allowed to warm to room temperature where it was stirred for 16 h. The reaction was quenched with a saturated solution of ammonium chloride and the phases were separated. The aqueous phase was then extracted with diethyl ether (3×20 mL) and the combined organic extracts were dried over Na₂SO₄, filtered and concentrated under reduced pressure. The concentrated material was then chromatographed (100% hexane→50% EtOAc-hexane) to obtain the chloromethylenation product XL^{α} (280 mg, 0.53 mmol, 48.9% yield).

### Step 5

Chloro intermediate XL^{α} (260 mg, 0.48 mmol) in THF (4 mL) was cooled to −78° C. under nitrogen. A solution of 1M LiHMDS solution in THF (0.5 mL, 0.5 mmol) was added slowly at −78° C. Upon completion of the addition, the reaction flask was allowed to warm to room temperature. After stirring at room temperature for 16 h, the reaction mixture was concentrated under vacuum and hexane (20 mL) was added. The precipitated lithium salts were filtered off through a Celite pad, rinsed with additional hexane and the combined filtrates were concentrated under vacuum to give crude bis(trimethylsilyl)amine product XLI^{α}.

### Step 6

To a stirred solution of 2-thiophenacetic acid (80 mg, 0.57 mmol) in DCM (10 mL) at 0° C. under nitrogen was added EDCI (137 mg, 0.72 mmol) and HOBT (77 mg, 0.57 mmol). After stirring at 0° C. for 30 minutes, a solution of the crude bis-silyl amide (XLI^{α}) intermediate in DCM (5 mL) followed by N-methyl-morpholine (0.1 mL, 0.98 mmol) were sequentially added at 0° C. Upon completion of the addition, the reaction flask was allowed to warm to room temperature. After stirring at room temperature overnight, the reaction mixture was washed with water, dried and concentrated under vacuum. The residue was purified by column chromatography (100% DCM→25% EtOAc/DCM) to afford amide XLII^{α} (100 mg, 0.157 mmol, 32.7% yield for 2 steps).

### Step 7

A solution of amide XLII^{α} (50 mg, 0.078 mmol) in anisole (2.5 mL) at 0° C. was treated with pre-cooled 90% aq trifluoroacetic acid (10 mL). The reaction mixture was warmed to room temperature and stirred for 16 h. The mixture was evaporated in vacuo, azeotroped with MeCN (3×5 mL). The residue was sonicated in water (10 mL) and ether (10 mL). The aqueous phase was separated, washed with ether (2×5 mL) and freeze dried to give fluffy solid 2-((3R)-2-hydroxy-3-(2-(thiophen-2-yl)acetamido)-1,5,2-dioxaborepan-7-yl)acetic acid 1^{α} (15 mg, 0.48 mmol, 61.4% yield). ¹H NMR (CD₃OD) δ ppm 6.98-7.00 (m, 1H), 7.00-7.09 (m, 1H), 7.33-7.35 (m, 1H); ESIMS found for C₁₂H₁₆BNO₆S m/z 296 (M−H₂O)⁺.

Illustrative compounds of Formula (II) are shown in Table 2. Some structures are shown with defined configurations at selected stereocenters but the shown stereochemistries are not meant to be limiting and all possible stereoisomers of the shown structures are to be considered encompassed herein. Compounds of any absolute and relative configurations at the stereocenters as well as mixtures of enantiomers and diastereoisomers of any given structure are also encompassed herein.

TABLE 2

| Ex- | Structure |
|---|---|
| ample | |
| 73 | 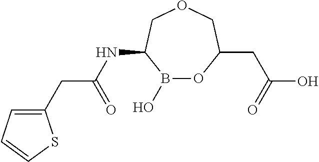 [SMILES: O=C(O)CC1COC[C@H](NC(=O)Cc2cccs2)B(O)O1] |
| 74 | 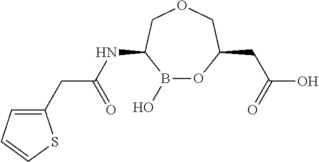 [SMILES: O=C(O)C[C@@H]1COC[C@H](NC(=O)Cc2cccs2)B(O)O1] |
| 75 | 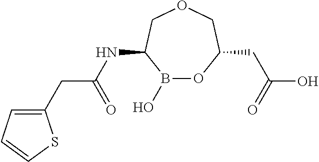 [SMILES: O=C(O)C[C@H]1COC[C@H](NC(=O)Cc2cccs2)B(O)O1] |
| 76 | 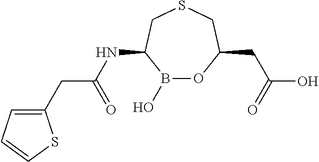 [SMILES: O=C(O)C[C@@H]1CSC[C@H](NC(=O)Cc2cccs2)B(O)O1] |
| 77 | 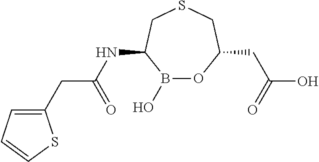 [SMILES: O=C(O)C[C@H]1CSC[C@H](NC(=O)Cc2cccs2)B(O)O1] |
| 78 | 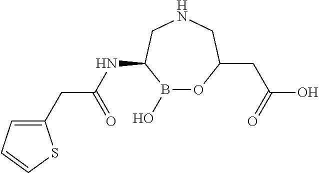 [SMILES: O=C(O)CC1CNC[C@H](NC(=O)Cc2cccs2)B(O)O1] |
| 79 | 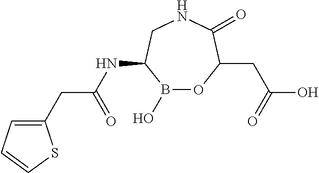 [SMILES: O=C(O)CC1OB(O)[C@@H](NC(=O)Cc2cccs2)CNC1=O] |
| 80 | 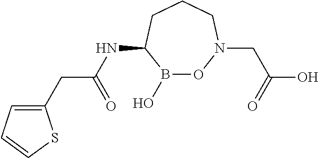 [SMILES: O=C(O)CN1CCC[C@H](NC(=O)Cc2cccs2)B(O)O1] |
| 81 | 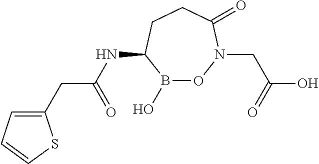 [SMILES: O=C(O)CN1OB(O)[C@@H](NC(=O)Cc2cccs2)CCC1=O] |
| 82 | 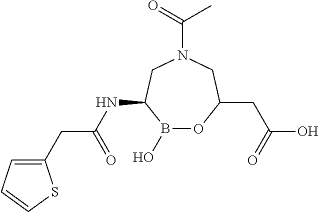 [SMILES: CC(=O)N1CC(CC(=O)O)OB(O)[C@@H](NC(=O)Cc2cccs2)C1] |
| 83 | 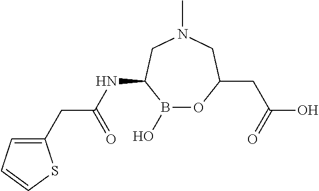 [SMILES: CN1CC(CC(=O)O)OB(O)[C@@H](NC(=O)Cc2cccs2)C1] |
| 84 | 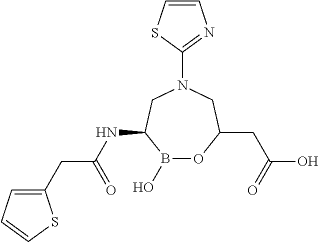 [SMILES: O=C(O)CC1CN(c2nccs2)C[C@H](NC(=O)Cc2cccs2)B(O)O1] |
| 85 | 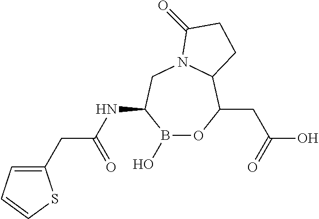 [SMILES: O=C(O)CC1OB(O)[C@@H](NC(=O)Cc2cccs2)CN2C(=O)CCC12] |
| 86 | 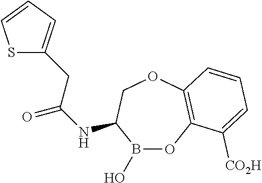 [SMILES: O=C(Cc1cccs1)N[C@H]1COc2cccc(C(=O)O)c2OB1O] |
| 87 | 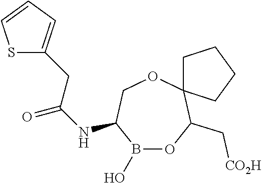 [SMILES: O=C(O)CC1OB(O)[C@@H](NC(=O)Cc2cccs2)COC12CCCC2] |
| 88 | 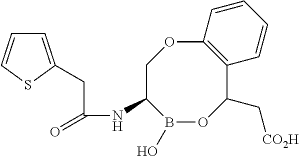 [SMILES: O=C(O)CC1OB(O)[C@@H](NC(=O)Cc2cccs2)COc2ccccc21] |
| 89 | 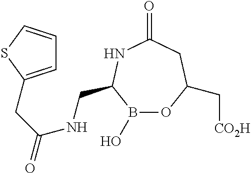 [SMILES: O=C(O)CC1CC(=O)N[C@H](CNC(=O)Cc2cccs2)B(O)O1] |
| 90 | 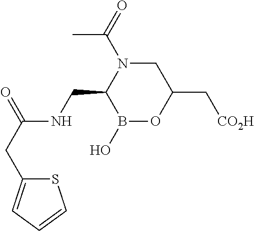 [SMILES: CC(=O)N1CC(CC(=O)O)OB(O)[C@H]1CNC(=O)Cc1cccs1] |
| 91 | 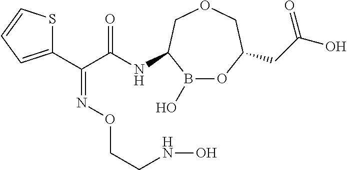 [SMILES: O=C(O)C[C@H]1COC[C@H](NC(=O)/C(=N\OCCNO)c2cccs2)B(O)O1] |
| 92 | 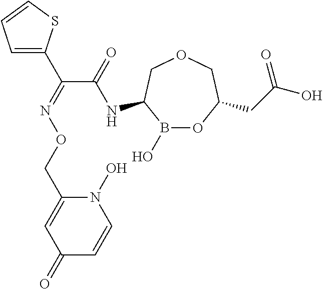 [SMILES: O=C(O)C[C@H]1COC[C@H](NC(=O)/C(=N\OCc2cc(=O)ccn2O)c2cccs2)B(O)O1] |
| 93 | 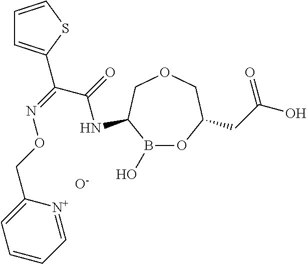 |
| 94 | 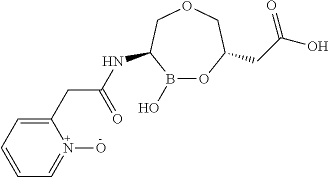 |
| 95 | 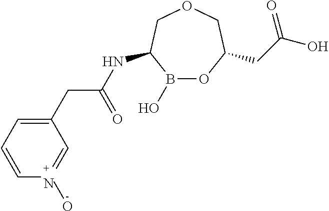 |
| 96 | 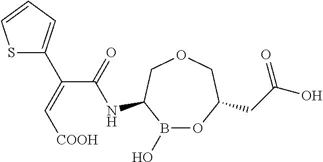 [SMILES: O=C(O)/C=C(\C(=O)N[C@H]1COC[C@H](CC(=O)O)OB1O)c1cccs1] |
| 97 | 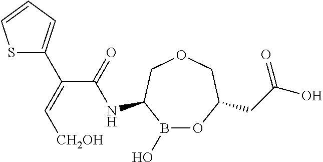 [SMILES: O=C(O)C[C@H]1COC[C@H](NC(=O)/C(=C\CO)c2cccs2)B(O)O1] |
| 98 | 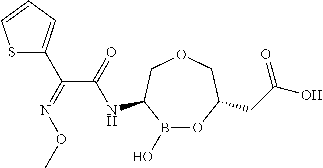 [SMILES: CO/N=C(\C(=O)N[C@H]1COC[C@H](CC(=O)O)OB1O)c1cccs1] |
| 99 | 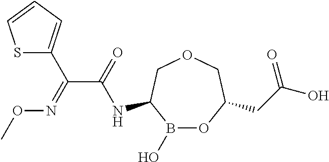 [SMILES: CO/N=C(/C(=O)N[C@H]1COC[C@H](CC(=O)O)OB1O)c1cccs1] |
| 100 | 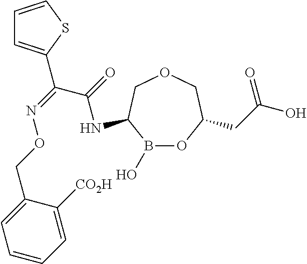 [SMILES: O=C(O)C[C@H]1COC[C@H](NC(=O)/C(=N\OCc2ccccc2C(=O)O)c2cccs2)B(O)O1] |
| 101 | 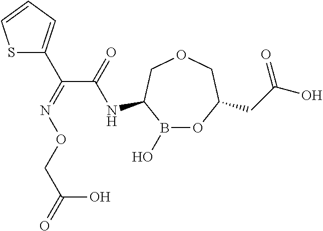 [SMILES: O=C(O)CO/N=C(\C(=O)N[C@H]1COC[C@H](CC(=O)O)OB1O)c1cccs1] |
| 102 | 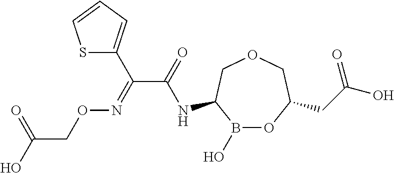 [SMILES: O=C(O)CO/N=C(/C(=O)N[C@H]1COC[C@H](CC(=O)O)OB1O)c1cccs1] |
| 103 | 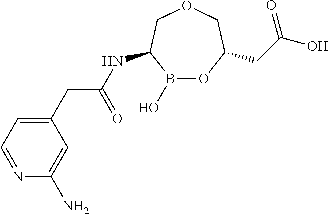 [SMILES: Nc1cc(CC(=O)N[C@H]2COC[C@H](CC(=O)O)OB2O)ccn1] |
| 104 | 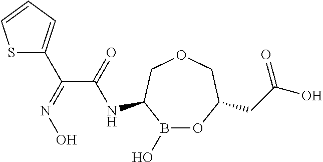 [SMILES: O=C(O)C[C@H]1COC[C@H](NC(=O)/C(=N\O)c2cccs2)B(O)O1] |
| 105 | 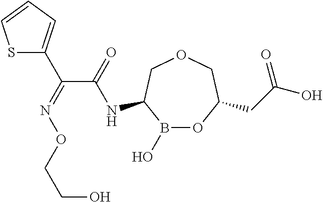 [SMILES: O=C(O)C[C@H]1COC[C@H](NC(=O)/C(=N\OCCO)c2cccs2)B(O)O1] |
| 106 | 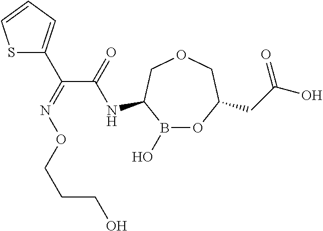 [SMILES: O=C(O)C[C@H]1COC[C@H](NC(=O)/C(=N\OCCCO)c2cccs2)B(O)O1] |
| 107 | 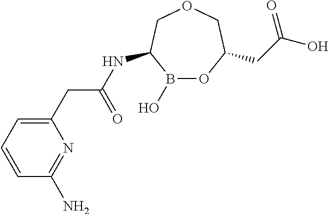 [SMILES: Nc1cccc(CC(=O)N[C@H]2COC[C@H](CC(=O)O)OB2O)n1] |
| 108 | 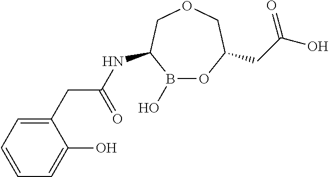 [SMILES: O=C(O)C[C@H]1COC[C@H](NC(=O)Cc2ccccc2O)B(O)O1] |
| 109 | 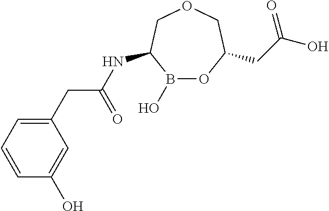 [SMILES: O=C(O)C[C@H]1COC[C@H](NC(=O)Cc2cccc(O)c2)B(O)O1] |
| 110 | 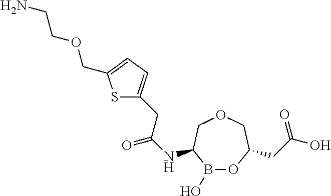 [SMILES: NCCOCc1ccc(CC(=O)N[C@H]2COC[C@H](CC(=O)O)OB2O)s1] |
| 111 | 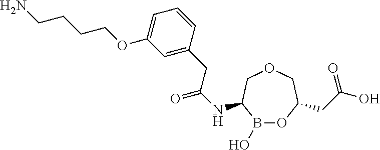 [SMILES: NCCCCOc1cccc(CC(=O)N[C@H]2COC[C@H](CC(=O)O)OB2O)c1] |
| 112 | 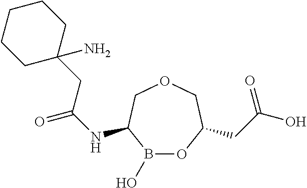 [SMILES: NC1(CC(=O)N[C@H]2COC[C@H](CC(=O)O)OB2O)CCCCC1] |
| 113 | 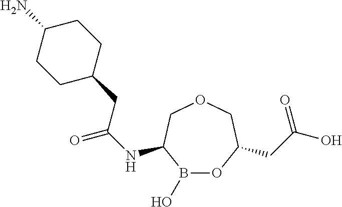 [SMILES: N[C@H]1CC[C@H](CC(=O)N[C@H]2COC[C@H](CC(=O)O)OB2O)CC1] |
| 114 | 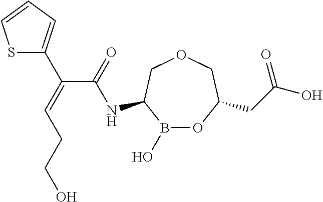 [SMILES: O=C(O)C[C@H]1COC[C@H](NC(=O)/C(=C\CCO)c2cccs2)B(O)O1] |
| 115 | 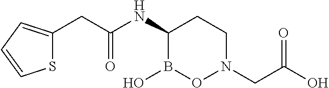 [SMILES: O=C(O)CN1CC[C@H](NC(=O)Cc2cccs2)B(O)O1] |
| 116 | 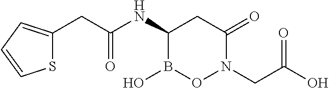 [SMILES: O=C(O)CN1OB(O)[C@@H](NC(=O)Cc2cccs2)CC1=O] |

### Example 11

The potency and spectrum of β-lactamase inhibitors was determined by assessing their antibiotic potentiation activity.

The potentiation effect is observed by the reduction of the minimum inhibitory concentration of β-lactam antibiotics in the presence of β-lactamase inhibitors (BLIs). The activity of BLIs in combination with biapenem is assessed by the checkerboard assay (Antimicrobial Combinations. In Antibiotics in Laboratory Medicine, Ed. Victor Lorian, M.D., Fourth edition, 1996, pp 333-338) using broth microdilution method performed as recommended by the CLSI (Clinical Laboratory Standards Institute) 2009. Methods for Dilution of Antimicrobial Susceptibility Tests for Bacteria That Grow Aerobically—Eighth Edition; Approved Standard. CLSI Document M07-A8, 2009). In this assay, multiple dilutions of two drugs, namely BLI and β-lactam (biapenem), are being tested, alone and in combination, at concentrations equal to, above and below their respective minimal inhibitory concentrations (MICs). BLIs are solubilized in 10% DMSO at 10 mg/mL. Stock solutions are further diluted, according to the needs of a particular assay, in Mueller Hinton Broth (MHB). Stock solution can be stored at −80° C.

The checkerboard (CB) assay is performed in microtiter plates. Biapenem is diluted in the x axis, each column containing a single concentration of antibiotic. BLIs are diluted in the y axis, each row containing an equal concentration of BLI. The result of these manipulations is that each well of the microtiter plate contains a unique combination of concentrations of the two agents. The assay is performed in MHB with a final bacterial inoculum of 5×105 CFU/mL (from an early-log phase culture). Microtiter plates are incubated during 20 h at 35° C. and are read using a microtiter plate reader (Molecular Devices) at 650 nm as well as visual observation using a microtiter plate reading mirror. The MIC is defined as the lowest concentration of antibiotics, within the combination, at which the visible growth of the organism is completely inhibited. Activity of BLIs is reported at MPC8, or the minimal potentiation concentration to reduce the MIC of antibiotic 8-fold.

Biapenem is a carbapenem β-lactam; only selected β-lactamases confer resistance to this class of antibiotics. Among them are serine carbapemenases that belong to class A and class D. Biapenem potentiation is studied in strains expressing various carbapenemases from these classes using CB assays. Various cyclic boronic acid derivatives showed significant potentiation of biapenem against the strains expressing class A carbapenemases: MPC8 (minimal potentiation concentration of cyclic boronic acid derivative (μg/mL) to reduce the MIC of Biapenem 8-fold) varied from 0.02 μg/mL to 0.16 μg/mL (Table 3). Cyclic boronic acid derivatives were capable of reducing biapenem MICs up to 1000-fold (Table 3).

TABLE 3

| Strain | Organism | Description | PCR | Class | Compound | MPC8 |
|---|---|---|---|---|---|---|
| ECL1004 | Enterobacter cloacae | Serine carbapenemase | NMC-A | A | 1 | Y |
| EC1007 | Escherichia coli | Serine carbapenemase | KPC-3 | A | 1 | X |
| KP1004 | Klebsiella pneumoniae | Serine carbapenemase | KPC-2 | A | 1 | Y |
| SM1000 | Serratia marcescens | Serine carbapenemase | SME-2 | A | 1 | Y |
| ECL1004 | Enterobacter cloacae | Serine carbapenemase | NMC-A | A | 2 | Y |
| EC1007 | Escherichia coli | Serine carbapenemase | KPC-3 | A | 2 | X |
| KP1004 | Klebsiella pneumoniae | Serine carbapenemase | KPC-2 | A | 2 | X |
| SM1000 | Serratia marcescens | Serine carbapenemase | SME-2 | A | 2 | Y |
| ECL1004 | Enterobacter cloacae | Serine carbapenemase | NMC-A | A | 3 | X |
| EC1007 | Escherichia coli | Serine carbapenemase | KPC-3 | A | 3 | X |
| KP1004 | Klebsiella pneumoniae | Serine carbapenemase | KPC-2 | A | 3 | X |
| KP1008 | Klebsiella pneumoniae | Serine carbapenemase | KPC-2 | A | 3 | X |
| SM1000 | Serratia marcescens | Serine carbapenemase | SME-2 | A | 3 | Y |
| AB1052 | Acinetobacter baumannii | OXA-carbapenemase | OXA-24 | D | 3 | Z |
| AB1054 | Acinetobacter baumannii | OXA-carbapenemase | OXA-23 | D | 3 | Z |
| AB1057 | Acinetobacter baumannii | OXA-carbapenemase | OXA-58 | D | 3 | Z |
| ECL1004 | Enterobacter cloacae | Serine carbapenemase | NMC-A | A | 4 | X |
| EC1007 | Escherichia coli | Serine carbapenemase | KPC-3 | A | 4 | X |
| KP1004 | Klebsiella pneumoniae | Serine carbapenemase | KPC-2 | A | 4 | X |
| KP1008 | Klebsiella pneumoniae | Serine carbapenemase | KPC-2 | A | 4 | X |
| SM1000 | Serratia marcescens | Serine carbapenemase | SME-2 | A | 4 | X |
| AB1052 | Acinetobacter baumannii | OXA-carbapenemase | OXA-24 | D | 4 | Z |
| AB1054 | Acinetobacter baumannii | OXA-carbapenemase | OXA-23 | D | 4 | Z |
| AB1057 | Acinetobacter baumannii | OXA-carbapenemase | OXA-58 | D | 4 | Z |
| ECL1004 | Enterobacter cloacae | Serine carbapenemase | NMC-A | A | 5 | Y |
| EC1007 | Escherichia coli | Serine carbapenemase | KPC-3 | A | 5 | X |
| KP1004 | Klebsiella pneumoniae | Serine carbapenemase | KPC-2 | A | 5 | X |
| SM1000 | Serratia marcescens | Serine carbapenemase | SME-2 | A | 5 | Y |
| AB1052 | Acinetobacter baumannii | OXA-carbapenemase | OXA-24 | D | 5 | Z |
| AB1054 | Acinetobacter baumannii | OXA-carbapenemase | OXA-23 | D | 5 | Z |
| AB1057 | Acinetobacter baumannii | OXA-carbapenemase | OXA-58 | D | 5 | Z |
| ECL1004 | Enterobacter cloacae | Serine carbapenemase | NMC-A | A | 6 | Y |
| EC1007 | Escherichia coli | Serine carbapenemase | KPC-3 | A | 6 | X |
| KP1004 | Klebsiella pneumoniae | Serine carbapenemase | KPC-2 | A | 6 | X |
| SM1000 | Serratia marcescens | Serine carbapenemase | SME-2 | A | 6 | Y |
| AB1052 | Acinetobacter baumannii | OXA-carbapenemase | OXA-24 | D | 6 | Z |
| AB1054 | Acinetobacter baumannii | OXA-carbapenemase | OXA-23 | D | 6 | X |
| AB1057 | Acinetobacter baumannii | OXA-carbapenemase | OXA-58 | D | 6 | Z |

X = MPC8 of less than 0.16 μg/mL.
Y = MPC8 of 0.16 μg/mL to 1 μg/mL.
Z = MPC8 of greater than 1 μg/mL.

### Example 12

The activity of β-lactamase inhibitors to inhibit hydrolysis of biapenem was studied. Lysates were prepared from bacteria expressing various β-lactamases as a source of enzymes. Bacterial lysates were prepared as follows. A single colony from the fresh overnight plate was transferred to 5 mL of LB broth and grown to OD₆₀₀=0.6-0.8. Next, this culture was transferred to 500 mL of LB and grown to OD₆₀₀=0.7-0.9. Cells were pelleted by centrifugation at 5000 RPM (JA-14 rotor) for 15 minutes at room temperature. The pellet was resuspended in 10 mL of PBS. Five freeze-thaw cycles by putting cells at −20° C. and thawing them at the room temperature were next applied. After the last thaw step cells were spun down at 18K for 30 minutes and the supernatant was collected. This lysate was stored at −20° C.

Next, the activity of bacterial lysates was optimized for biapenem cleavage as follows. 50 μl of buffer A (50 mM Sodium Phosphate pH=7; 0.5% glucose, 1 mM MgCl₂) was added to each well of 96-well UV-transparent plate. 50 μl of lysate was titrated vertically in 96-well plate column to generate 2-fold lysate dilutions. 100 μl of buffer A was added to each well, placed in plate reader at 37° C. and incubated for 15 minutes. 50 μl of 50 μg/mL solutions of biapenem in buffer A (pre-incubated at 37° C. for 15 minutes) were added to each well. Hydrolysis of biapenem was measured at 296 nm. This experiment was used to determine the optimal lysate dilution which produced a linear curve of relative UV signal that decreased to approximately OD=0.3-0.5 over 1 hour.

Finally, the potency of cyclic boronic acid derivative to inhibit the cleavage of biapenem cleavage by bacterial lysates was determined. 100 μl of buffer A (50 mM Sodium Phosphate pH=7; 0.5% glucose, 1 mM MgCl₂) was added to each well of 96-well UV-transparent plate. 50 μl of 6× cyclic boronic acid derivative solution in buffer A was titrated vertically in 96-well plate column to generate 3-fold dilutions. 50 μl of diluted lysate in buffer A (optimal dilution is determined in experiment above) was added, and the plate was incubated in the plate reader at 37° C. for 15 minutes. 50 μl of 50 μg/mL solution of biapenem in buffer A (pre-incubated at 37° C. for 15 minutes) were next added to each well and hydrolysis of biapenem was recorded at 296 nm. EC₅₀ of inhibition was determined by plotting the rate of biapenem cleavage vs. cyclic boronic acid derivative concentration.

The results of these experiments are presented in Table 4. These experiments demonstrate that the described compounds are inhibitors with a broad-spectrum activity towards various β-lactamases.

TABLE 4

| | | | | | IC₅₀ (μg/mL) of inhibition of biapenem | | | | | |
|---|---|---|---|---|---|---|---|---|---|---|
| | | | | | hydrolysis | | | | | |
| Strain | Organism | Description | PCR | Class | Tazobactam | 3 | 4 | 5 | 6 | 7 |
| EC1007 | Escherichia coli | Serine | KPC-3 | A | Z | Y | Y | X | X | Z |
| | | carbapenemase | | | | | | | | |
| KP1004 | Klebsiella | Serine | KPC-2 | A | Z | Z | Y | X | Y | ND |
| | pneumoniae | carbapenemase | | | | | | | | |
| KP1008 | Klebsiella | Serine | KPC-2 | A | Z | Z | Z | Y | Y | ND |
| | pneumoniae | carbapenemase | | | | | | | | |
| SM1000 | Serratia | Serine | SME-2 | A | Y | Z | Y | X | Y | Z |
| | marcescens | carbapenemase | | | | | | | | |

X = IC₅₀ of less than 0.1 μg/mL.
Y = IC₅₀ of 0.1 μg/mL to 1 μg/mL.
Z = IC₅₀ of greater than 1 μg/mL.
ND = Not Determined.

The potency and spectrum of β-lactamase inhibitors is also determined by assessing their biapenem potentiation activity in a dose titration potentiation assay using strains expressing serine carbapemenases (such as KPC). The potentiation effect is observed as the ability of BLI compounds to inhibit growth in the presence of sub-inhibitory concentration of biapenem. MIC of test strains vary from 4 μg/mL to >1 μg/mL. Biapenem is present in the test medium at 1 μg/mL. Compounds tested at the highest concentration of 40 μg/mL. In this assay potency of compounds is determined as a concentration of BLIs to inhibit growth of bacteria in the presence of 1 μg/mL of biapenem (MPC₁). Table 5 summarizes BLI potency of biapenem potentiation (MPC₁). Biapenem MIC for each strain is also shown.

TABLE 5

Biapenem MIC

| | >8 | 8 | 4 | 8 |
|---|---|---|---|---|
| | BPM | BPM | BPM | BPM |
| | MPC₁ | MPC₁ | MPC₁ | MPC₁ |
| | KP1004 | KP1008 | EC1007 | ECL1004 |
| | KPC-2 | KPC-2 | KPC-3 | NMC-A |
| Tazobactam | 40 | 0.3 | 5 | 0.6 |
| 3 | X | X | X | Y |
| 4 | X | X | X | X |
| 5 | X | X | X | X |
| 6 | X | X | X | X |
| 33 | X | X | X | X |
| 34 | X | X | X | Y |
| 35 | X | X | X | Y |
| 36 | Z | X | Y | X |
| 37 | X | X | X | X |
| 38 | X | X | X | X |
| 39 | X | X | X | X |
| 40 | Y | X | Y | Y |
| 41 | X | X | X | Y |
| 42 | X | X | X | Y |
| 43 | X | X | X | X |
| 44 | X | X | X | Y |
| 45 | Y | X | X | Z |
| 46 | X | X | X | X |
| 47 | Y | X | X | Z |
| 48 | X | X | X | X |
| 49 | X | X | X | X |
| 50 | X | X | X | X |
| 51 | X | X | X | Y |
| 52 | X | X | X | Y |
| 53 | X | X | X | Y |
| 54 | X | X | X | X |
| 55 | X | X | X | X |
| 56 | X | X | X | X |
| 57 | X | X | X | X |
| 58 | Z | Z | z | Z |
| 59 | Y | X | X | X |
| 60 | X | X | X | X |
| 61 | X | X | x | X |
| 62 | X | X | X | X |
| 63 | Y | X | Y | Y |
| 64 | Y | X | X | X |
| 65 | Y | X | Y | Z |
| 66 | X | X | X | X |

X = MPC₁ of less than 1 μg/mL.
Y = MPC₁ of 1 μg/mL to 5 μg/mL.
Z = MPC₁ of greater than 5 μg/mL.
ND = Not Determined.

### Example 13

Checkerboard assays were used to evaluate the ability of Compound 5 to potentiate biapenem against the strains expressing KPC alone or in combination with additional beta-lactamases. The highest concentration of Compound 5 was 10 mg/L. The results are present in the Table 6. Compound 5 was capable to significantly potentiate biapenem.

TABLE 6

| | | Concentration of Compound 5 (mg/L) to potentiate biapenem (mg/L) | | | | | | | | | |
|---|---|---|---|---|---|---|---|---|---|---|---|
| Organism | Strain | Enzyme | Antibiotic | 0 | 0.16 | 0.31 | 0.625 | 1.25 | 2.5 | 5 | 10 |
| Klebsiella | KP1004 | KPC-2 | Biapenem | Z | X | X | X | X | X | X | X |
| pneumoniae | | | | | | | | | | | |
| Klebsiella | KP1008 | KPC-2 | Biapenem | Z | X | X | X | X | X | NG | NG |
| pneumoniae | | | | | | | | | | | |
| Klebsiella | KP1082 | KPC-2, | Biapenem | Y | X | X | X | X | X | X | X |
| pneumoniae | | SHV-1 | | | | | | | | | |
| Klebsiella | KP1087 | KPC-2, | Biapenem | Z | Z | Z | Z | Y | Y | X | X |
| pneumoniae | | CTX-M- | | | | | | | | | |
| | | 15, SHV- | | | | | | | | | |
| | | 11, TEM-1 | | | | | | | | | |
| Klebsiella | KX1019 | KPC-2, | Biapenem | Z | Y | Y | Y | Y | Y | X | X |
| oxytoca | | OXA-2 | | | | | | | | | |
| Klebsiella | KX1017 | KPC-2, | Biapenem | Y | Y | Y | X | X | X | X | X |
| oxytoca | | OXA-2, | | | | | | | | | |
| | | SHV-30 | | | | | | | | | |
| Klebsiella | KX1018 | KPC-2, | Biapenem | Z | X | X | X | X | X | NG | NG |
| oxytoca | | SHV-40, | | | | | | | | | |
| | | OXY-1 | | | | | | | | | |
| Escherichia | EC1007 | KPC-3 | Biapenem | Z | X | X | X | X | X | X | X |
| coli | | | | | | | | | | | |
| Enterobacter | ECL1058 | KPC-3, | Biapenem | Z | Y | Y | Y | X | X | X | X |
| cloacae | | SHV-11, | | | | | | | | | |
| | | TEM-1 | | | | | | | | | |
| Enterobacter | ECL1059 | KPC-3, | Biapenem | Y | X | X | X | X | X | X | X |
| cloacae | | SHV-12, | | | | | | | | | |
| | | TEM-1 | | | | | | | | | |
| Klebsiella | KP1083 | KPC-3, | Biapenem | Z | Y | X | X | X | X | X | X |
| pneumoniae | | SHV-1, | | | | | | | | | |
| | | TEM-1 | | | | | | | | | |
| Klebsiella | KP1084 | KPC-3, | Biapenem | Z | Z | Z | Z | Z | Y | X | X |
| pneumoniae | | SHV-11, | | | | | | | | | |
| | | TEM-1 | | | | | | | | | |
| Klebsiella | KP1088 | KPC-3, | Biapenem | Z | Y | X | X | X | X | X | X |
| pneumoniae | | SHV-11, | | | | | | | | | |
| | | TEM-1 | | | | | | | | | |

X = MIC of less than 0.5 mg/L.
Y = MIC of 0.5 mg/L to 4 mg/L.
Z = MIC of greater than 4 mg/L.
NG = No Growth.

### Example 14

The β-lactamase inhibitor, Compound 5, was tested for its ability to potentiate Biapenem in bacterial strains expressing the metallo-β-lactamase NDM-1, the serine β-lactamase KPC-2, and both β-lactamases. MIC values for each bacterial strain were measured for Biapenem at various concentrations of Compound 5. Table 7 summarizes the activity of Compound 5 in combination with Biapenem. The presence of Compound 5 decreased the Biapenem MIC in bacterial strains that expressed the beta-lactamase, KPC-2 alone or in combination with NDM-1.

TABLE 7

| | | Biapenem MIC (μg/ml) | | |
|---|---|---|---|---|
| | | Escherichia | Klebsiella | Klebsiella |
| | | coli | pneumonia | pneumonia |
| Organism | | (EC1061) | (KP1004) | (KPM1097) |
| (strain) | | Donor | Recipient | Trans- |
| Description | | strain | strain | conjugant |
| Beta-lactamase | | NDM-1 | KPC-2 | NDM-1, KPC-2 |
| Compound | 0 | 0.125 | 8 | 16 |
| 5 | 0.31 | 0.13 | 0.5 | 2 |
| (μg/ml) | 0.63 | 0.13 | 0.25 | 1 |
| | 1.25 | 0.125 | 0.25 | 1 |
| | 2.5 | 0.06 | 0.06 | 1 |
| | 5 | 0.125 | 0.06 | 1 |
| | 10 | 0.06 | ≦0.03 | 1 |
| | 20 | 0.06 | ≦0.03 | 1 |

### Example 15

The two β-lactamase inhibitors (BLIs), Compound 5 and Compound 68, were tested for their ability to potentiate the two antimicrobial compounds, Aztreonam and Tigemonan, in bacterial strains expressing the β-lactamases: NDM-1, CMY-6, SHV-11, CTX-M-15, and TEM-1. MIC values for each bacterial strain were measured for each antimicrobial compound at various concentrations of each BLI. Table 8 summarizes the activity of each BLI in combination with Aztreonam or Tigemonan. The presence of each BLI decreased the Aztreonam or Tigemonan MICs in bacterial strains that expressed the beta-lactamase, NDM-1 and CMY-6.

TABLE 8

| | | | Antibiotic MIC (μg/ml) | | | | | | | |
|---|---|---|---|---|---|---|---|---|---|---|
| Organism/Strain | | | in the presence of BLI (μg/ml) | | | | | | | |
| (β-lactamase) | Antibiotic | BLI | 0 | 0.31 | 0.63 | 1.25 | 2.5 | 5 | 10 | 20 |
| E. coli./EC1061 | Aztreonam | Compound 5 | 16 | 4 | 4 | 2 | 2 | 0.5 | 0.5 | 0.5 |
| (NDM-1 CMY-6) | | | | | | | | | | |
| E. coli./EC1061 | Tigemonam | Compound 5 | 8 | ND | 1 | 1 | 1 | 0.5 | 0.5 | 1 |
| (NDM-1 CMY-6) | | | | | | | | | | |
| E. coli./EC1061 | Tigemonam | Compound | 16 | 0.5 | 0.5 | 0.5 | 0.5 | 0.5 | 0.5 | ND |
| (NDM-1 CMY-6) | | 68 | | | | | | | | |
| K. pneumoniae/ | Aztreonam | Compound 5 | 256 | 256 | 128 | 32 | 16 | 8 | 4 | 2 |
| KP1081 | | | | | | | | | | |
| (NDM-1, CMY- | | | | | | | | | | |
| 6, SHV-11, CTX- | | | | | | | | | | |
| M-15, TEM-1) | | | | | | | | | | |
| K. pneumoniae/ | Tigemonam | Compound 5 | 16 | ND | 4 | 4 | 4 | 4 | 4 | 4 |
| KP1081 | | | | | | | | | | |
| (NDM-1,CMY- | | | | | | | | | | |
| 6, SHV-11, CTX- | | | | | | | | | | |
| M-15, TEM-1) | | | | | | | | | | |
| K. pneumoniae/ | Tigemonam | Compound | 16 | 8 | 8 | 4 | 4 | 4 | 4 | ND |
| KP1081 CNDM-1,CMY- | | 68 | | | | | | | | |
| 6, SHV-11, CTX- | | | | | | | | | | |
| M-15, TEM-1) | | | | | | | | | | |

### Example 16

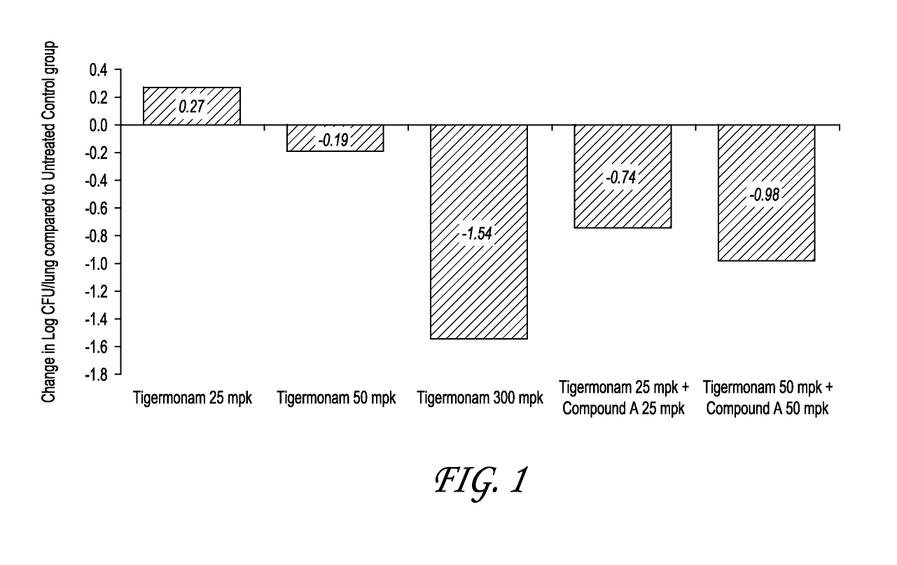

Tigemonnam was administered by IP route with and without the BLI, Compound A (also known as Compound 68) in a neutropenic mouse thigh infection model. The infection comprised E. coli EC1061 (contains NDM-1 and CMY-6, as shown in Table 8). The results are summarized in FIG. 1. Tigemonam MIC=8 mg/L; 0.5 mg/L with 0.31 μg/ml Compound A (also known as Compound 68).

While the present invention has been described in some detail for purposes of clarity and understanding, one skilled in the art will appreciate that various changes in form and detail can be made without departing from the true scope of the invention.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-44. (canceled)

45. A method of increasing sensitivity of a bacterial infection to treatment with an antimicrobial β-lactam compound resistant to degradation by a metallo β-lactamase, wherein the antimicrobial compound resistant to degradation by a metallo β-lactamase has a minimum inhibitory concentration for E. coli expressing the metallo β-lactamase less than about 0.05 μg/ml, said method comprising:
identifying a bacterial infection as including bacteria that comprises a serine β-lactamase and the metallo β-lactamase; and
contacting said bacteria with an effective amount of a β-lactamase inhibitor, wherein the β-lactamase inhibitor is selected from the group consisting of: 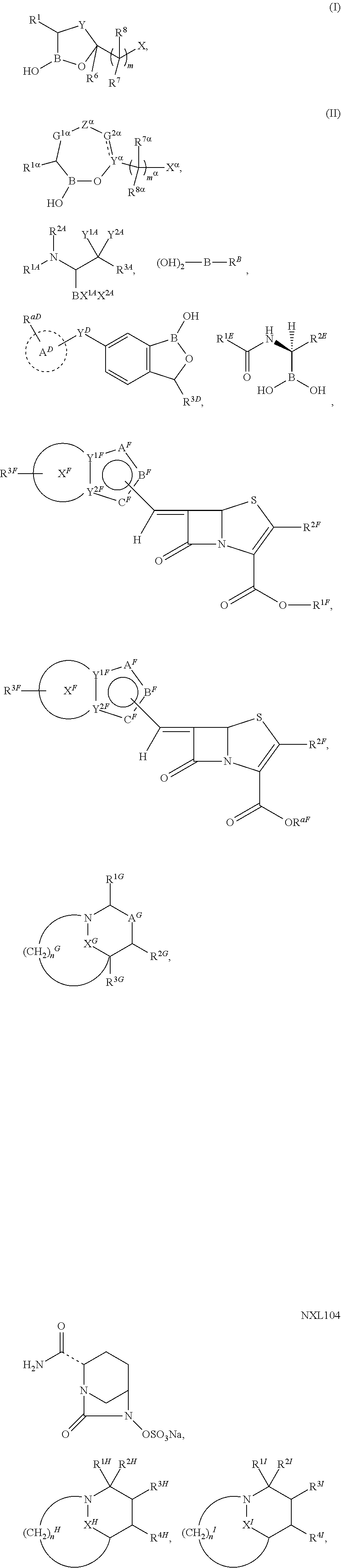 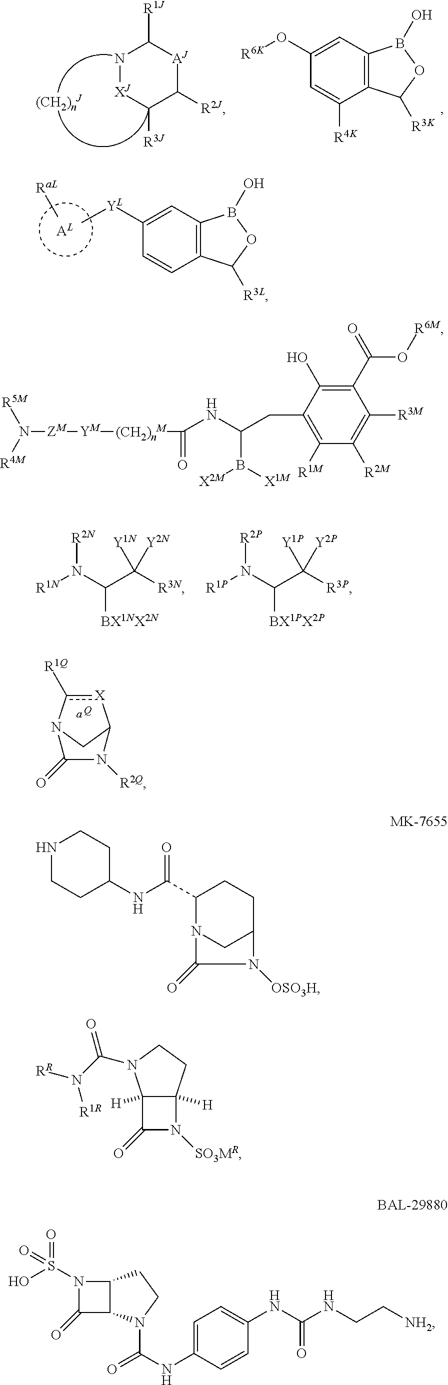 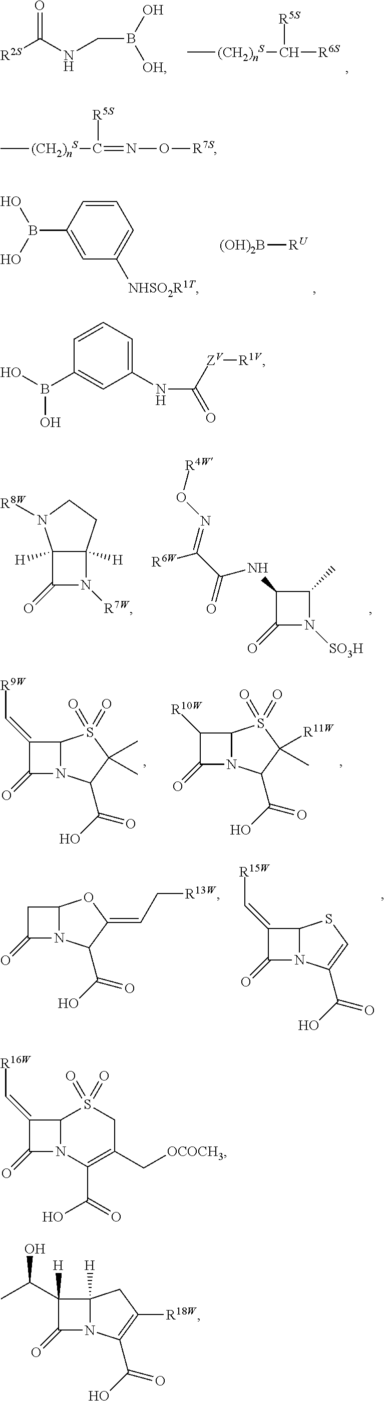 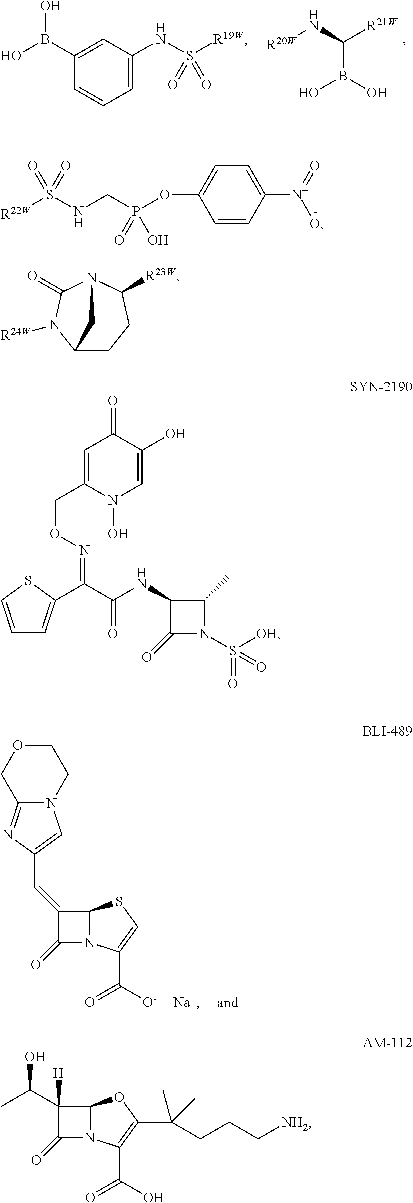
or pharmaceutically acceptable salt thereof,
wherein each substituent is defined in the specification.

46. (canceled)

47. A method of treating a bacterial infection that includes bacteria comprising a serine β-lactamase and a metallo β-lactamase, said method comprising:
contacting said bacteria with a β-lactamase inhibiting effective amount of a β-lactamase inhibitor and an antibacterially effective amount of an antimicrobial β-lactam compound resistant to degradation by a metallo β-lactamase, wherein the antimicrobial compound resistant to degradation by the metallo β-lactamase has a minimum inhibitory concentration for E. coli expressing the metallo β-lactamase less than about 0.05 μg/ml, wherein the β-lactamase inhibitor is selected from the group consisting of: 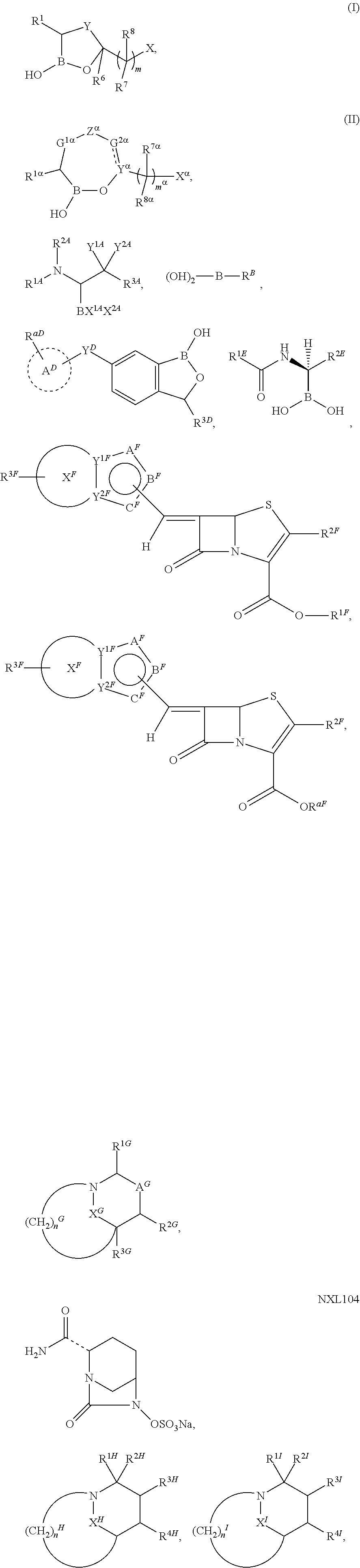 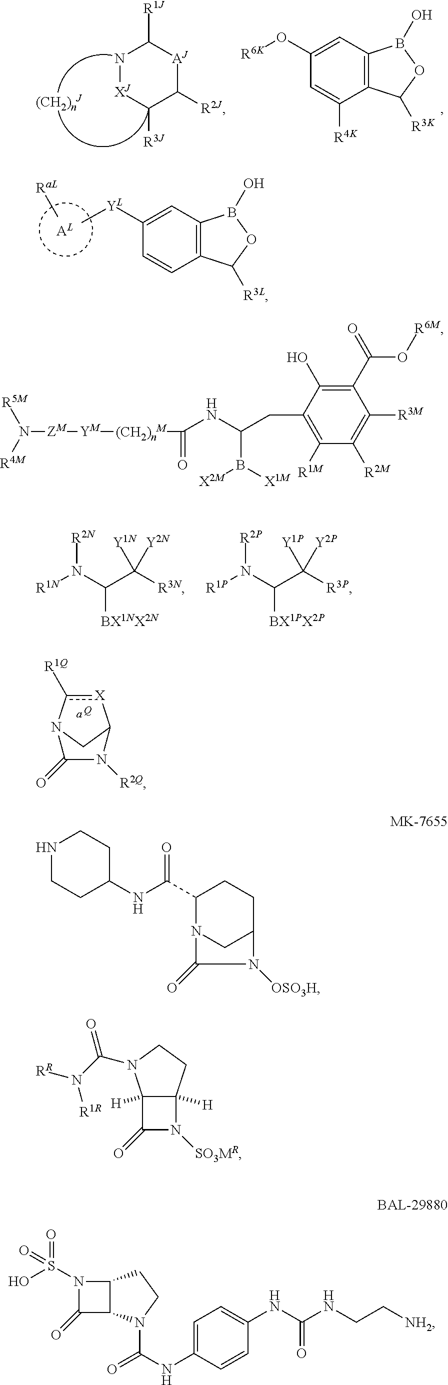 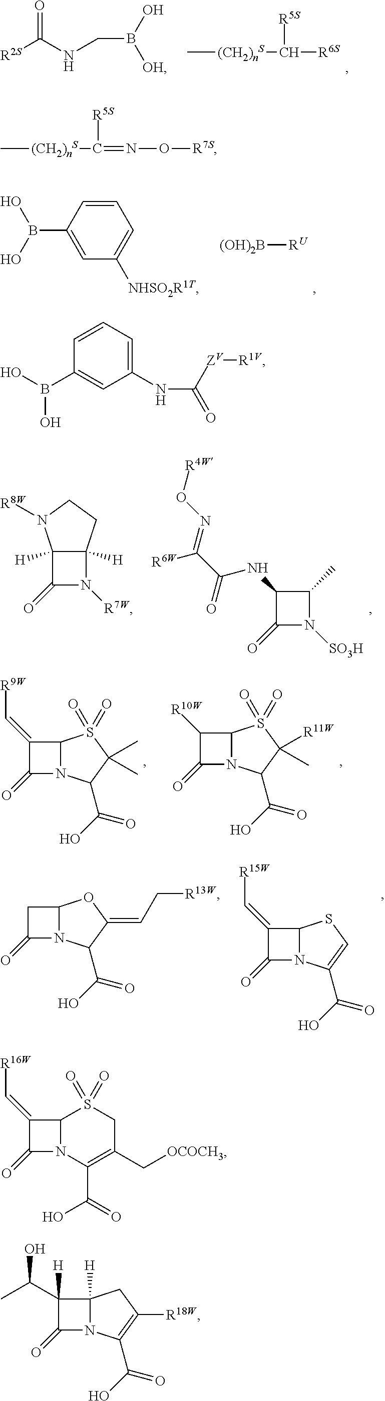 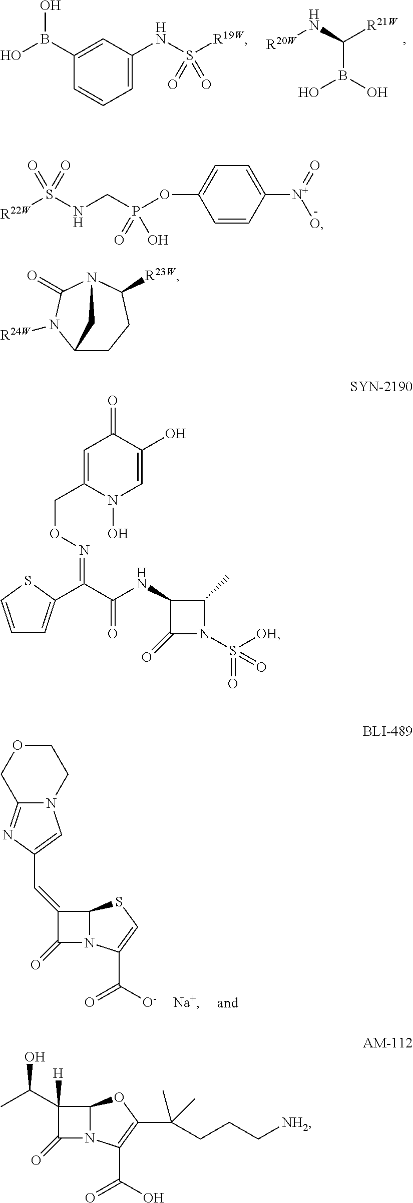
or pharmaceutically acceptable salt thereof,
wherein each substituent is defined in the specification.

48. The method of claim 47, further comprising identifying said bacterial infection as including bacteria that comprises a serine β-lactamase and a metallo β-lactamase.

49. The method of claim 47, wherein contacting said bacteria with a β-lactamase inhibiting effective amount of a β-lactamase inhibitor and an antibacterially effective amount of an antimicrobial β-lactam compound resistant to degradation by a metallo β-lactamase comprises administering the β-lactamase inhibitor and the antimicrobial compound resistant to degradation by a metallo β-lactamase to a subject having said bacterial infection.

50. The method of claim 49, wherein said administering comprises administering a pharmaceutical composition comprising said β-lactamase inhibitor and said antimicrobial compound resistant to degradation by a metallo β-lactamase to said subject.

51. (canceled)

52. (canceled)

53. (canceled)

54. A method of increasing sensitivity of a bacterial infection to treatment with an antimicrobial β-lactam compound resistant to degradation by a metallo β-lactamase, wherein the sensitivity to the antimicrobial compound resistant to degradation by the metallo β-lactamase of the bacteria contacted with the β-lactamase inhibitor increases at least about 2-fold compared to bacteria not contacted with the β-lactamase inhibitor, said method comprising:
identifying a bacterial infection as including bacteria that comprises a serine β-lactamase and the metallo β-lactamase; and
contacting said bacteria with an effective amount of a β-lactamase inhibitor, wherein the β-lactamase inhibitor is selected from the group consisting of: 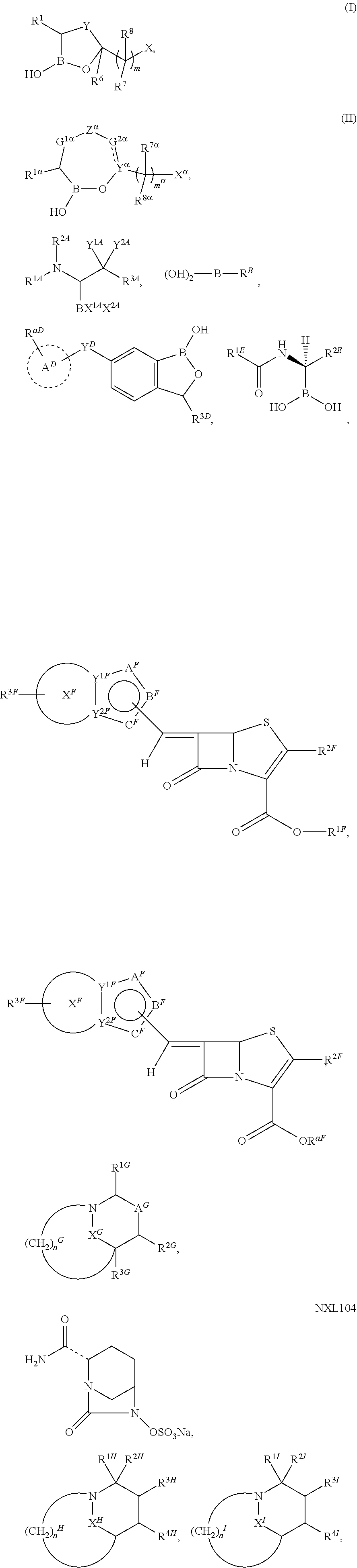 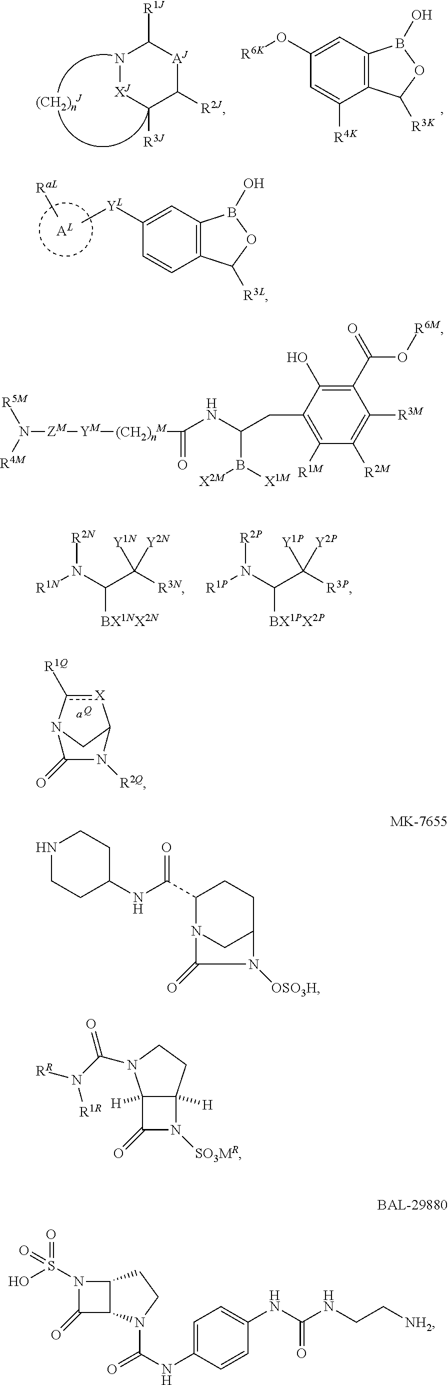 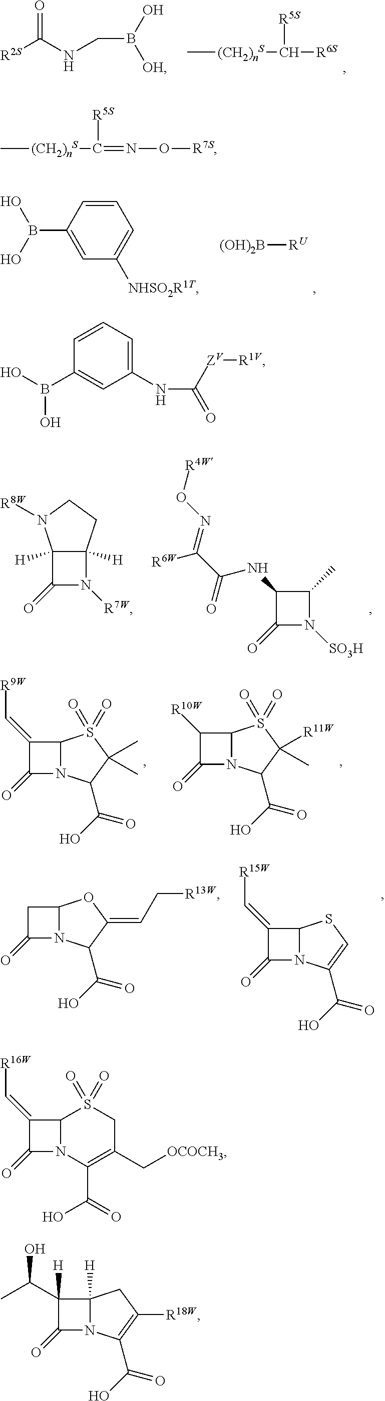 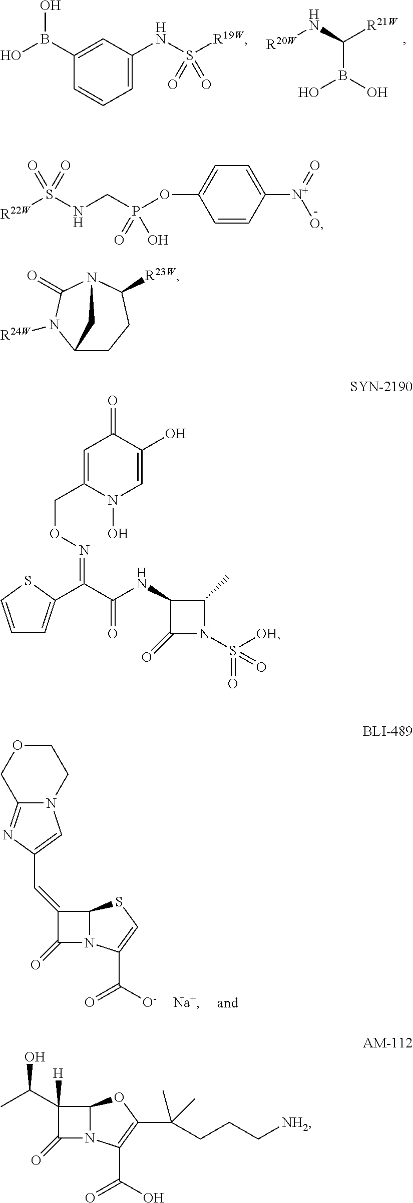
or pharmaceutically acceptable salt thereof,
wherein each substituent is defined in the specification.

55. (canceled)

56. The method of claim 47,
wherein the sensitivity to the antimicrobial compound resistant to degradation by the metallo β-lactamase of the bacteria contacted with the β-lactamase inhibitor increases at least about 2-fold compared to bacteria not contacted with the β-lactamase inhibitor.

57. (canceled)

58. (canceled)

59. (canceled)

60. (canceled)

61. (canceled)

62. (canceled)

63. The method of claim 47, wherein the antimicrobial compound resistant to degradation by a metallo β-lactamase has a Kₘ for the metallo β-lactamase greater than about 100 μM.

64. (canceled)

65. The method of claim 47, wherein the antimicrobial compound resistant to degradation by a metallo β-lactamase has a minimum inhibitory concentration for E. coli expressing the metallo β-lactamase less than about 250 μg/ml.

66. The method of claim 47, wherein the antimicrobial compound resistant to degradation by a metallo β-lactamase comprises biapenem.

67. The method of claim 47, wherein the antimicrobial compound resistant to degradation by a metallo β-lactamase comprises a monobactam.

68. The method of claim 47, wherein the antimicrobial compound resistant to degradation by a metallo β-lactamase is selected from the group consisting of Aztreonam, Tigemonam, Carumonam, SYN-2416, BAL30072, and Nocardicin A.

69. (canceled)

70. The method of claim 47, wherein the sensitivity to the antimicrobial compound resistant to degradation by a metallo β-lactamase of the bacteria contacted with the β-lactamase inhibitor increases at least about 4-fold compared to bacteria not contacted with the β-lactamase inhibitor.

71. The method of claim 47, wherein the serine β-lactamase is selected from the group consisting of NMC-A, SME, KPC-2, OXA-48, and KPC-3.

72. The method of claim 47, wherein the serine β-lactamase comprises a KPC enzyme.

73. The method of claim 47, wherein the serine β-lactamase comprises KPC-2.

74. The method of claim 47, wherein the metallo β-lactamase comprises NDM-1.

75. The method of claim 47, wherein the metallo β-lactamase comprises IMP, VIM, SPM, and GIM.

76. The method of claim 47, wherein the bacterial infection comprises a bacterium selected from the group consisting of Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus.

77. (canceled)

78. (canceled)

79. The method of claim 47, wherein the β-lactamase inhibitor is: 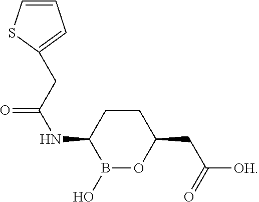 [SMILES: O=C(O)C[C@@H]1CC[C@H](NC(=O)Cc2cccs2)B(O)O1]
